(19) United States
(12) Patent Application Publication
(10) Pub. No.: US 2023/0065814 A1
(43) Pub. Date: Mar. 2, 2023

(54) CONJUGATES OF-ELECTRON-PAIR-DONATING HETEROAROMATIC NITROGEN-COMPRISING COMPOUNDS

(71) Applicant: Ascendis Pharma A/S, Hellerup (DK)

(72) Inventors: Nicola BISEK, Heidelberg (DE); Samuel WEISBROD, Heidelberg (DE)

(21) Appl. No.: 17/596,831

(22) PCT Filed: Jun. 19, 2020
(86) PCT No.: PCT/EP2020/067149

§ 371 Date: Dec. 20, 2021

(30) Foreign Application Priority Data
Jun. 21, 2019 (EP) 19181790.7

(51) Int. Cl. A61K 47/69; A61K 31/416; A61K 31/4439; A61K 47/54; A61K 47/60; A61K 47/64; A61P 35/00; C07D 231/56; C07D 401/06; C07D 403/06

(52) U.S. Cl. CPC A61K 47/6903; A61K 31/416; A61K 31/4439; A61K 47/542; A61K 47/60; A61K 47/6455; A61P 35/00; C07D 231/56; C07D 401/06; C07D 403/06

(57) ABSTRACT

The present invention relates to conjugates of π-electron-pair-donating heteroaromatic nitrogen-comprising drugs and pharmaceutically acceptable salts thereof, pharmaceutical compositions comprising said conjugates and the use of said conjugates as medicaments.

The present invention relates to conjugates of π-electron-pair-donating heteroaromatic nitrogen-comprising drugs and pharmaceutically acceptable salts thereof, pharmaceutical compositions comprising said conjugates and the use of said conjugates as medicaments.

To improve physicochemical or pharmacokinetic properties, such as the in vivo circulation half-life of drugs, such drugs can be conjugated to a carrier, such as a polymer. Typically, polymers in drug delivery are either used in a non-covalent complexation of the drug and polymer, embedding of drug in a polymer or by covalent conjugation of the drug to a polymeric moiety.

However, the non-covalent approach requires a highly efficient drug encapsulation to prevent uncontrolled, burst-type release of the drug due to the disintegration of the drug-polymer complex after administration. Restraining the diffusion of an unbound, water-soluble drug molecule requires strong van der Waals contacts, frequently mediated through hydrophobic moieties and charged moieties for electrostatic binding. Many conformationally sensitive drugs, such as proteins or peptides, are rendered dysfunctional during the complexation process and/or during subsequent storage of the non-covalently bound drug.

Alternatively, a drug may be covalently conjugated to a polymeric moiety via a stable linker or a reversible linker from which the drug is released. If the drug is stably conjugated to the polymeric moiety, such conjugate needs to exhibit sufficient residual activity to have a pharmaceutical effect and thus the conjugate is constantly in an active form.

One advantage of conjugating a drug to a polymeric moiety through a reversible linker is that no residual activity of the conjugate is needed, because the drug exhibits its pharmacological effect upon release from the conjugate. A conjugate may exhibit no or little drug activity, i.e. the conjugate is pharmacologically inactive. This approach is applied to all classes of molecules, from so-called small molecules, through natural products up to large proteins. The drug of such a conjugate may be released by enzymatic or non-enzymatic cleavage of the linkage between the polymeric moiety and the drug moiety or by a combination of both. However, enzyme-dependence is usually less preferred, because enzyme levels may vary significantly between patients what makes the correct dosing difficult.

WO 2005/099768 A2, WO 2009/095479 A2 and WO 2016/196124 A2 disclose carrier-linked prodrugs whereby drug moieties are reversibly connected to transient linkers via amines such as aliphatic amines, by formation of for example, amide bonds. Such aliphatic amines consist of only hydrogen and alkyl substituents. WO 2011/012722 A1 discloses carrier-linked prodrugs, whereby the drug moieties are attached via their aromatic amines to reversible linkers through formation of amide bonds. Such aromatic amines comprise an aromatic ring to which the nitrogen atom of the amine is attached, meaning that the nitrogen atom of aromatic amines is not part of the aromatic ring system. Although the beforementioned patent applications report the ability of converting drug moieties that comprise aliphatic and aromatic amines into conjugates, they do not explore the ability of explicitly employing π-electron-pair-donating heteroaromatic nitrogens of drug molecules as linkage points to reversible linkers. Such moieties are usually good leaving groups, so the expectation is that any polymer conjugated to such moiety via the aforementioned linkers may be cleaved off too rapidly to provide any meaningful half-life extension. Therefore, there is still a need for conjugates, in which the linker attachment takes place at the π-electron-pair-donating heteroaromatic nitrogens.

WO 2008/076225 A2 discloses prodrugs of non-nucleoside reverse transcriptase inhibitors, whereby a linker moiety is attached to one of the nitrogen atoms that is located within an indazole ring. These prodrugs are converted into their corresponding drugs by hydrolysis of a urea bond or cyclization, such as nucleophilic addition of an amine to the urea bond at physiological pH (e.g. a pH of greater than about 7). However, WO 2008/076225 A2 does not disclose attaching drugs to polymeric moieties and thus does not teach how to improve the pharmacokinetics and therapeutic index of drugs by reversibly and covalently conjugating said drugs to polymeric moieties via reversible linkers. Therefore, said conjugates do not, for example, significantly extend the circulation half-life of drugs.

It is thus an object of the present invention to at least partially overcome the shortcomings described above.

This object is achieved with a conjugate or a pharmaceutically acceptable salt thereof comprising at least one moiety -D conjugated via at least one moiety -L¹-L²- to at least one moiety Z, wherein a moiety -L¹- is conjugated to a π-electron-pair-donating heteroaromatic N of a moiety -D and wherein the linkage between -D and -L¹- is reversible and wherein a moiety -L²- is conjugated to Z, wherein
each -D is independently a π-electron-pair-donating heteroaromatic N-comprising moiety of a drug D-H;
each -L²- is independently a single bond or a spacer moiety;
each Z is independently a polymeric moiety or a C₈₋₂₄ alkyl;
each -L¹- is independently a linker moiety of formula (I):

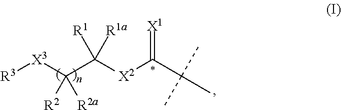

wherein
the dashed line indicates the attachment to the π-electron-pair-donating heteroaromatic N of -D;
n is an integer selected from the group consisting of 0, 1, 2, 3 and 4;
═X¹ is selected from the group consisting of ═O, ═S and ═N(R⁴);
—²— is selected from the group consisting of —O—, —S—, —N(R⁵)— and —C(R⁶)(R^{6a})—;
—X³— is selected from the group consisting of

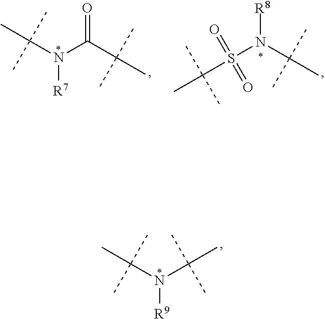

—C(R¹⁰)(R^{10a})—, —C(R¹¹)(R^{11a})—C(R¹²)(R^{12a})—, —O— and —C(O)—;
   R¹, —R^{1a}, —R⁶, —R^{6a}, —R¹⁰, —R^{10a}, —R¹¹, —R^{11a}, —R¹², —R^{12a} and each of —R² and —R^{2a} are independently selected from the group consisting of —H, —C(O)OH, halogen, —CN, —OH, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R¹³, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a})—, —S—, —N(R¹⁴)—, —OC(OR¹⁴)(R^{14a})—, —N(R¹⁴)C(O)N(R^{14a})— and —OC(O)N(R¹⁴)—;
   —R³, —R⁴, —R⁵, —R⁷, —R⁸ and —R⁹ are independently selected from the group consisting of —H, -T, —CN, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R¹³, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a}—, —S—, —N(R¹⁴)—, —OC(OR¹⁴)(R^{14a})—, —N(R¹⁴)C(O)N(R^{14a})— and —OC(O)N(R¹⁴)—;
      each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl; wherein each T is independently optionally substituted with one or more —R¹³, which are the same or different;
      wherein —R¹³ is selected from the group consisting of —H, —NO₂, —OCH₃, —CN, —N(R¹⁴)(R^{14a}), —OH, —C(O)OH and C₁₋₆ alkyl; wherein C₁₋₆ alkyl is optionally substituted with one or more halogen, which are the same or different;
      wherein —R¹⁴ and —R^{14a} are independently selected from the group consisting of —H and C₁₋₆ alkyl; wherein C₁₋₆ alkyl is optionally substituted with one or more halogen, which are the same or different;
   optionally, one or more of the pairs —R¹/—R^{1a}, —R²/—R^{2a}, two adjacent —R², —R⁶/—R^{6a}, —R¹⁰/—R^{10a}, —R¹¹/—R^{11a}, —R¹²/—R^{12a} and —R³/—R⁹ are joined together with the atom to which they are attached to form a C₃₋₁₀ cycloalkyl, 3- to 10-membered heterocyclyl or an 8- to 11-membered heterobicyclyl;
   optionally, one or more of the pairs —R¹/—R², —R¹/—R⁵, —R¹/—R⁶, —R¹/—R⁹, —R¹/—R¹⁰, —R²/—R⁵, —R³/—R^{6a}, —R⁴/—R⁵, —R⁴/—R⁶, —R⁵/—R¹⁰, —R⁶/—R¹⁰ and —R¹¹/—R¹² are joined together with the atoms to which they are attached to form a ring -A-;
      wherein -A- is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl;
   optionally, —R¹ and an adjacent —R² form a carbon-carbon double bond provided that n is selected from the group consisting of 1, 2, 3 and 4;
   optionally, two adjacent —R² form a carbon-carbon double bond provided that n is selected from the group consisting of 2, 3 and 4;
   provided that if —X²— is —N(R⁵)—, —X³— is selected from the group consisting of

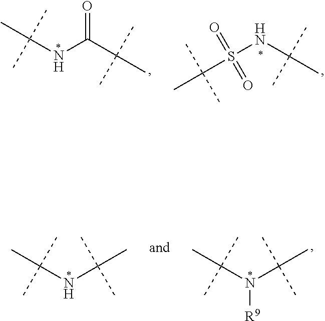

and the distance between the nitrogen atom marked with an asterisk and the carbon atom marked with an asterisk in formula (I) is 5, 6 or 7 atoms and if present the carbon-carbon double bond formed between —R¹ and —R² or two adjacent —R² is in a cis configuration; and
each -L¹- is substituted with -L²- and optionally further substituted.

In certain embodiments, the conjugate of the present invention or a pharmaceutically acceptable salt thereof comprises at least one moiety -D conjugated via at least one moiety -L¹-L²- to at least one moiety Z, wherein a moiety -L¹- is conjugated to a π-electron-pair-donating heteroaromatic N of a moiety -D and wherein the linkage between -D and -L¹- is reversible and wherein a moiety -L²- is conjugated to Z, wherein
each -D is independently a π-electron-pair-donating heteroaromatic N-comprising moiety of a drug D-H;
each -L²- is independently a single bond or a spacer moiety;
each Z is independently a polymeric moiety or a C₈₋₂₄ alkyl;
each -L¹- is independently a linker moiety of formula (I):

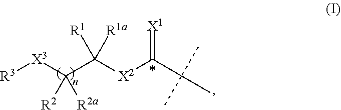

wherein
the dashed line indicates the attachment to the π-electron-pair-donating heteroaromatic N of -D;
n is an integer selected from the group consisting of 0, 1, 2, 3 and 4;
═X¹ is selected from the group consisting of ═O, ═S and ═N(R⁴);
—X²— is selected from the group consisting of —O—, —S—, —N(R⁵)— and —C(R⁶)(R^{6a})—;
X³— is selected from the group consisting of

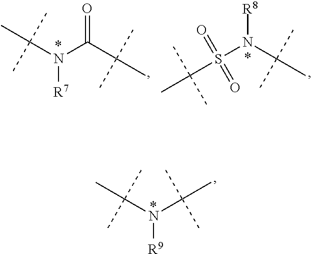

—C(R¹⁰)(R^{10a})—, —C(R¹¹)(R^{11a})—C(R¹²)(R^{12a})—, —O— and —C(O)—;
   —¹, —R^{1a}, —R⁶, —R^{6a}, —R¹⁰, —R^{10a}, —R¹¹, —R^{11a}, —R¹², —R^{12a} and each of —R² and —R^{2a} are independently selected from the group consisting of —H, —C(O)OH, halogen, —CN, —OH, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R¹³, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a})—, —S—, —N(R¹⁴)—, —OC(OR¹⁴)(R^{14a})—, —N(R¹⁴)C(O)N(R^{14a})— and —OC(O)N(R¹⁴)—;
   —R³, —R⁴, —R⁵, —R⁷, —R⁸ and —R⁹ are independently selected from the group consisting of —H, -T, —CN, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R¹³, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a}—), —S—, —N(R¹⁴)—, —OC(OR¹⁴)(R^{14a})—, —N(R¹⁴)C(O)N(R^{14a})— and —OC(O)N(R¹⁴)—;
      each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl; wherein each T is independently optionally substituted with one or more —R¹³, which are the same or different;
      wherein —R¹³ is selected from the group consisting of —H, —NO₂, —OCH₃, —CN, —N(R¹⁴)(R^{14a}), —OH, —C(O)OH and C₁₋₆ alkyl; wherein C₁₋₆ alkyl is optionally substituted with one or more halogen, which are the same or different;
      wherein —R¹⁴ and —R^{14a} are independently selected from the group consisting of —H and C₁₋₆ alkyl; wherein C₁₋₆ alkyl is optionally substituted with one or more halogen, which are the same or different;
   optionally, one or more of the pairs —R¹/—R^{1a}, —R²/—R^{2a}, two adjacent —R², —R⁶/—R^{6a}, —R¹⁰/—R^{10a}, —R¹¹/—R^{11a} and —R¹²/—R^{12a} joined together with the atom to which they are attached to form a C₃₋₁₀ cycloalkyl, 3- to 10-membered heterocyclyl or an 8- to 11-membered heterobicyclyl;
   optionally, one or more of the pairs —R¹/—R², —R¹/—R⁵, —R¹/—R⁶, —R¹/—R⁹, —R¹/—R¹⁰, —R³/—R^{6a}, —R⁴/—R⁵, —R⁴/—R⁶, —R⁵/—R¹⁰ and —R⁶/—R¹⁰ are joined together with the atoms to which they are attached to form a ring -A-;
      wherein -A- is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl;
   optionally, —R¹ and an adjacent —R² form a carbon-carbon double bond provided that n is selected from the group consisting of 1, 2, 3 and 4;
   optionally, two adjacent —R² form a carbon-carbon double bond provided that n is selected from the group consisting of 2, 3 and 4;
   provided that if —X²— is —N(R⁵)—, —X³— is selected from the group consisting of

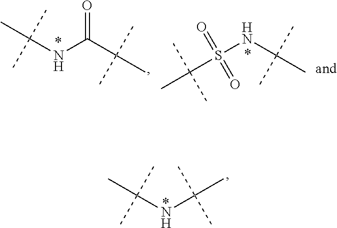

and the distance between the nitrogen atom marked with an asterisk and the carbon atom marked with an asterisk in formula (I) is 5, 6 or 7 atoms and if present the carbon-carbon double bond formed between —R¹ and —R² or two adjacent —R² is in a cis configuration; and
each -L¹- is substituted with -L²- and optionally further substituted.

It was surprisingly found that the reversible linker moiety -L¹- of formula (I) has advantageous properties, such as providing suitable release half-lives for drug moieties that are attached at one of their π-electron-pair-donating heteroaromatic nitrogen to said reversible linker moiety. This is surprising as π-electron-pair-donating heteroaromatic nitrogen-comprising moieties may be expected to be good leaving groups that would result in half-lives that are unsuitable for reducing the frequency of drug administration. It was also surprisingly found that despite having such good leaving groups within the conjugates of the present invention, applicants were able to identify conditions for their stable storage.

Within the meaning of the present invention the terms are used as follows.

As used herein, the term “a π-electron-pair-donating heteroaromatic N-comprising moiety” refers to the moiety which after cleavage of the linkage between -D and -L¹- results in a drug D-H and wherein the drug moiety -D and analogously the corresponding D-H comprises at least one, such as one, two, three, four, five, six, seven, eight, nine or ten heteroaromatic nitrogen atoms that donate a π-electron pair to the aromatic π-system. Examples of chemical structures comprising such heteroaromatic nitrogens that donate a π-electron pair to the aromatic λ-system include, but are not limited to, pyrrole, pyrazole, imidazole, isoindazole, indole, indazole, purine, tetrazole, triazole and carbazole. For example, in the imidazole ring below the heteroaromatic nitrogen which donates a π-electron pair to the aromatic π-system is marked with “#”.

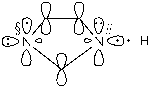

The π-electron-pair-donating heteroaromatic nitrogen atoms do not comprise heteroaromatic nitrogen atoms which only donate one electron (i.e. not a pair of π-electrons) to the aromatic ═-system, such as for example the nitrogen that is marked with “§” in the abovementioned imidazole ring structure. The drug D-H may exist in one or more tautomeric forms, such as with one hydrogen atom moving between at least two heteroaromatic nitrogen atoms. In all such cases, the linker moiety is covalently and reversibly attached at a heteroaromatic nitrogen that donates a π-electron pair to the aromatic π-system.

As used herein, the term “drug” refers to a substance used in the treatment, cure, prevention or diagnosis of a disease or used to otherwise enhance physical or mental well-being of a patient. If a drug is conjugated to another moiety, the moiety of the resulting product that originated from the drug is referred to as “drug moiety”.

As used herein, the term “moiety” means a part of a molecule, which lacks one or more atom(s) compared to the corresponding reagent. If, for example, a reagent of the formula “H—X—H” reacts with another reagent and becomes part of the reaction product, the corresponding moiety of the reaction product has the structure “H—X—” or “—X—”, whereas each “—” indicates attachment to another moiety. Accordingly, a drug moiety is released from a reversible linkage as a drug.

It is understood that if a sequence or chemical structure of a group of atoms is provided which group of atoms is attached to two moieties or is interrupting a moiety, said sequence or chemical structure can be attached to the two moieties in either orientation, unless explicitly stated otherwise. For example, a moiety “—C(O)N(R^{x})—” may be attached to two moieties or interrupting a moiety either as “—C(O)N(R^{x})—” or as “—N(R^{x})C(O)—”. Similarly, a moiety:

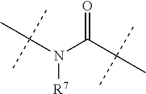

may be attached to two moieties or may interrupt a moiety either as:

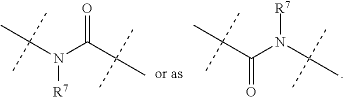

As used herein, the term “reagent” means a chemical compound, which comprises at least one functional group for reaction with the functional group of another chemical compound or drug. It is understood that a drug comprising a functional group is also a reagent.

It is recognized by one of ordinary skill in the art that the conjugates of the present invention are prodrugs. As used herein, the term “prodrug” refers to a drug moiety, that is reversibly and covalently conjugated to a polymeric moiety, such as Z, through at least one -L¹-L²- moiety. A prodrug releases the reversibly and covalently bound drug moiety -D in the form of its corresponding drug D-H. In other words, a prodrug is a conjugate comprising a drug moiety, which is covalently and reversibly conjugated to a polymeric moiety via at least one -L¹-L²-moiety. Such prodrugs or conjugates release the formerly conjugated drug moiety in the form of a free drug.

As used herein, the term “reversible linkage” or “biodegradable linkage” is a linkage that is cleavable, in the absence of enzymes under physiological conditions, which are aqueous buffer at pH 7.4 and 37° C., with a half-life ranging from one hour to six months, such as from one hour to four months, such as from one hour to three months, from one hour to two months or from one hour to one month. It is understood, however, that a reversible linkage may also be cleavable at other conditions, such as for example at a different pH or at a different temperature with a half-life ranging from one hour to six months, but that a test for determining reversibility is performed in the above-described physiological conditions (aqueous buffer, pH 7.4, 37° C.). Accordingly, a “stable linkage” is a linkage having a half-life under physiological conditions of more than six months.

As used herein, the term “stable” and “stability” with regards to a pharmaceutical formulation or composition comprising a conjugate of the present invention means that after a storage time, such as after one month, two months, four months, six months, eight months, twelve months, eighteen months, twenty-four months, thirty-six months, in particular after the indicated storage time, the pharmaceutical formulation or composition comprises less than 5% of the drug in its free form.

As used herein, the term “reversible linker moiety” is a moiety which is covalently conjugated to a drug moiety through a reversible linkage and which is also covalently conjugated to a moiety Z via a moiety -L²-. In certain embodiments, the linkage between Z and -L²- is a stable linkage.

As used herein, the term “about” in combination with a numerical value is used to indicate a range ranging from and including the numerical value plus and minus no more than 10% of said numerical value, in certain embodiments, no more than 8% of said numerical value, in certain embodiments, no more than 5% of said numerical value and in certain embodiments, no more than 2% of said numerical value. For example, the phrase “about 200” is used to mean a range ranging from and including 200+/−10%, i.e. ranging from and including 180 to 220; in certain embodiments, 200+/−8%, i.e. ranging from and including 184 to 216; in certain embodiments, ranging from and including 200+/−5%, i.e. ranging from and including 190 to 210; and in certain embodiments 200+/−2%, i.e. ranging from and including 196 to 204. It is understood that a percentage given as “about 20%” does not mean “20%+/−10%”, i.e. ranging from and including 10 to 30%, but “about 20%” means ranging from and including 18 to 22%, i.e. plus and minus 10% of the numerical value which is 20.

As used herein, the term “C₁₋₄ alkyl” alone or in combination means a straight-chain or branched alkyl moiety having 1 to 4 carbon atoms. If present at the end of a molecule, examples of straight-chain or branched C₁₋₄ alkyl are methyl, ethyl, n-propyl, isopropyl, n-butyl, isobutyl, sec-butyl and tert-butyl. When two moieties of a molecule are linked by the C₁₋₄ alkyl, then examples for such C₁₋₄ alkyl groups are —CH₂—, —CH₂—CH₂—, —CH(CH₃)—, —CH₂—CH₂—CH₂—, —CH(C₂H₅)—, —C(CH₃)₂—. Each hydrogen of a C₁₋₄ alkyl carbon may optionally be replaced by a substituent as defined below. Optionally, a C₁₋₄ alkyl may be interrupted by one or more moieties as defined below.

As used herein, the term “C₁₋₆ alkyl” alone or in combination means a straight-chain or branched alkyl moiety having 1 to 6 carbon atoms. If present at the end of a molecule, examples of straight-chain and branched C₁₋₆ alkyl groups are methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. When two moieties of a molecule are linked by the C₁₋₆ alkyl group, then examples for such C₁₋₆ alkyl groups are —CH₂—, —CH₂—CH₂—, —CH(CH₃)—, —CH₂—CH₂—CH₂—, —CH(C₂H₅)— and —C(CH₃)₂—. Each hydrogen atom of a C₁₋₆ carbon may optionally be replaced by a substituent as defined below. Optionally, a C₁₋₆ alkyl may be interrupted by one or more moieties as defined below.

Accordingly, “C₁₋₁₀ alkyl”, “C₁₋₂₀ alkyl”, “C₈₋₂₄ alkyl” or “C₁₋₅₀ alkyl” means an alkyl chain having 1 to 10, 1 to 20, 8 to 24 or 1 to 50 carbon atoms, respectively, wherein each hydrogen atom of the C₁₋₁₀, C₁₋₂₀, C₈₋₂₄ or C₁₋₅₀ carbon may optionally be replaced by a substituent as defined below. Optionally, a C₁₋₁₀ alkyl, C₁₋₂₀ alkyl, C₈₋₂₄ alkyl or C₁₋₅₀ alkyl may be interrupted by one or more moieties as defined below.

As used herein, the term “C₂₋₆ alkenyl” alone or in combination means a straight-chain or branched hydrocarbon moiety comprising at least one carbon-carbon double bond having 2 to 6 carbon atoms. If present at the end of a molecule, examples are —CH═CH₂, —CH═CH—CH₃, —CH₂—CH═CH₂, —CH═CHCH₂—CH₃ and —CH═CH—CH═CH₂. When two moieties of a molecule are linked by the C₂₋₆ alkenyl group, then an example of such C₂₋₆ alkenyl is —CH═CH—. Each hydrogen atom of a C₂₋₆ alkenyl moiety may optionally be replaced by a substituent as defined below. Optionally, a C₂₋₆ alkenyl may be interrupted by one or more moieties as defined below.

Accordingly, the terms “C₂₋₁₀ alkenyl”, “C₂₋₂₀ alkenyl” or “C₂₋₅₀ alkenyl” alone or in combination mean a straight-chain or branched hydrocarbon moiety comprising at least one carbon-carbon double bond having 2 to 10, 2 to 20 or 2 to 50 carbon atoms, respectively. Each hydrogen atom of a C₂₋₁₀ alkenyl, C₂₋₂₀ alkenyl or C₂₋₅₀ alkenyl group may optionally be replaced by a substituent as defined below. Optionally, a C₂₋₁₀ alkenyl, C₂₋₂₀ alkenyl or C₂₋₅₀ alkenyl may be interrupted by one or more moieties as defined below.

As used herein, the term “C₂₋₆ alkynyl” alone or in combination means a straight-chain or branched hydrocarbon moiety comprising at least one carbon-carbon triple bond having 2 to 6 carbon atoms. If present at the end of a molecule, examples are —C≡CH, —CH₂—C≡CH, CH₂—CH₂—C≡CH and CH₂—C≡C—CH₃. When two moieties of a molecule are linked by the alkynyl group, then an example is —C≡C—. Each hydrogen atom of a C₂₋₆ alkynyl group may optionally be replaced by a substituent as defined below. Optionally, one or more double bond(s) may occur. Optionally, a C₂₋₆ alkynyl may be interrupted by one or more moieties as defined below.

Accordingly, as used herein, the term “C₂₋₁₀ alkynyl”, “C₂₋₂₀ alkynyl” and “C₂₋₅₀ alkynyl” alone or in combination means a straight-chain or branched hydrocarbon moiety comprising at least one carbon-carbon triple bond having 2 to 10, 2 to 20 or 2 to 50 carbon atoms, respectively. Each hydrogen atom of a C₂₋₁₀ alkynyl, C₂₋₂₀ alkynyl or C₂₋₅₀ alkynyl group may optionally be replaced by a substituent as defined below. Optionally, one or more double bond(s) may occur. Optionally, a C₂₋₁₀ alkynyl, C₂₋₂₀ alkynyl or C₂₋₅₀ alkynyl may be interrupted by one or more moieties as defined below.

As mentioned above, a C₁₋₄ alkyl, C₁₋₆ alkyl, C₁₋₁₀ alkyl, C₁₋₂₀ alkyl, C₁₋₅₀ alkyl, C₈₋₂₄ alkyl, C₂₋₆ alkenyl, C₂₋₁₀ alkenyl, C₂₋₂₀ alkenyl, C₂₋₅₀ alkenyl, C₂₋₆ alkynyl, C₂₋₁₀ alkynyl, C₂₋₂₀ alkenyl or C₂₋₅₀ alkynyl may optionally be interrupted by one or more moieties which in certain embodiments are selected from the group consisting of

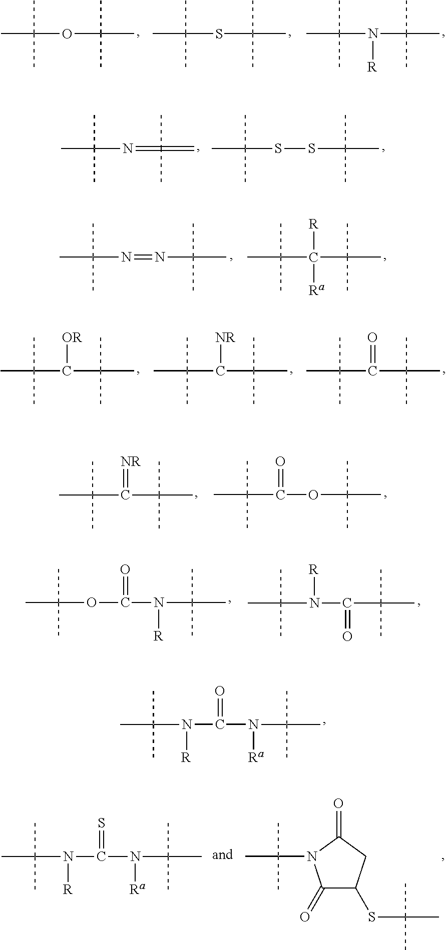

wherein
dashed lines indicate attachment to the remainder of the moiety or reagent;
—R and —R^{a} are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and which moieties and linkages are optionally further substituted.

As used herein, the term “C₃₋₁₀ cycloalkyl” means a cyclic alkyl chain having 3 to 10 carbon atoms, which may be saturated or unsaturated, e.g. cyclopropyl, cyclobutyl, cyclopentyl, cyclohexyl, cyclohexenyl, cycloheptyl, cyclooctyl, cyclononyl or cyclodecyl. Each hydrogen atom of a C₃₋₁₀ cycloalkyl carbon may be replaced by a substituent as defined below. The term “C₃₋₁₀ cycloalkyl” also includes bridged bicycles like norbornane or norbornene.

As used herein, the term “8- to 30-membered carbopolycyclyl” or “8- to 30-membered carbopolycycle” means a cyclic moiety of two or more rings with 8 to 30 ring atoms, where two neighboring rings share at least one ring atom and that may contain up to the maximum number of double bonds (aromatic or non-aromatic ring which is fully, partially or un-saturated). In certain embodiments, an 8- to 30-membered carbopolycyclyl means a cyclic moiety of two, three, four or five rings. In certain embodiments, an 8- to 30-membered carbopolycyclyl means a cyclic moiety of two, three or four rings.

As used herein, the term “3- to 10-membered heterocyclyl” or “3- to 10-membered heterocycle” means a ring with 3, 4, 5, 6, 7, 8, 9 or 10 ring atoms that may contain up to the maximum number of double bonds (aromatic or non-aromatic ring which is fully, partially or un-saturated) wherein at least one ring atom up to 4 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom.

Examples for 3- to 10-membered heterocycles include but are not limited to aziridine, oxirane, thiirane, azirine, oxirene, thiirene, azetidine, oxetane, thietane, furan, thiophene, pyrrole, pyrroline, imidazole, imidazoline, pyrazole, pyrazoline, oxazole, oxazoline, isoxazole, isoxazoline, thiazole, thiazoline, isothiazole, isothiazoline, thiadiazole, thiadiazoline, tetrahydrofuran, tetrahydrothiophene, pyrrolidine, imidazolidine, pyrazolidine, oxazolidine, isoxazolidine, thiazolidine, isothiazolidine, thiadiazolidine, sulfolane, pyran, dihydropyran, tetrahydropyran, imidazolidine, pyridine, pyridazine, pyrazine, pyrimidine, piperazine, piperidine, morpholine, tetrazole, triazole, triazolidine, tetrazolidine, diazepane, azepine and homopiperazine. Each hydrogen atom of a 3- to 10-membered heterocyclyl or 3- to 10-membered heterocyclic group may be replaced by a substituent as defined below.

As used herein, the term “8- to 11-membered heterobicyclyl” or “8- to 11-membered heterobicycle” means a heterocyclic moiety of two rings with 8 to 11 ring atoms, where at least one ring atom is shared by both rings and that may contain up to the maximum number of double bonds (aromatic or non-aromatic ring which is fully, partially or un-saturated) wherein at least one ring atom up to 6 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for an 8- to 11-membered heterobicycle are indole, indoline, benzofuran, benzothiophene, benzoxazole, benzisoxazole, benzothiazole, benzisothiazole, benzimidazole, benzimidazoline, quinoline, quinazoline, dihydroquinazoline, quinoline, dihydroquinoline, tetrahydroquinoline, decahydroquinoline, isoquinoline, decahydroisoquinoline, tetrahydroisoquinoline, dihydroisoquinoline, benzazepine, purine and pteridine. The term 8- to 11-membered heterobicycle also includes spiro structures of two rings like 1,4-dioxa-8-azaspiro[4.5]decane or bridged heterocycles like 8-aza-bicyclo[3.2.1]octane. Each hydrogen atom of an 8- to 11-membered heterobicyclyl or 8- to 11-membered heterobicycle carbon may be replaced by a substituent as defined below.

Similarly, the term “8- to 30-membered heteropolycyclyl” or “8- to 30-membered heteropolycycle” means a heterocyclic moiety of more than two rings with 8 to 30 ring atoms, in certain embodiments of three, four or five rings, where two neighboring rings share at least one ring atom and that may contain up to the maximum number of double bonds (aromatic or non-aromatic ring which is fully, partially or unsaturated), wherein at least one ring atom up to 10 ring atoms are replaced by a heteroatom selected from the group of sulfur (including —S(O)—, —S(O)₂—), oxygen and nitrogen (including ═N(O)—) and wherein the ring is linked to the rest of a molecule via a carbon or nitrogen atom.

It is understood that the phrase “the pair —R^{x}/—R^{y} is joined together with the atom to which they are attached to form a C₃₋₁₀ cycloalkyl, 3- to 10-membered heterocyclyl or an 8- to 11-membered heterobicyclyl” in relation with a moiety of the structure:

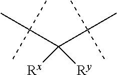

means that R^{x} and R^{y} form the following structure:

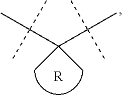

wherein R is C₃₋₁₀ cycloalkyl, 3- to 10-membered heterocyclyl or an 8- to 11-membered heterobicyclyl.

It is also understood that the phrase “the pair —R^{x}/—R^{y} is joined together with the atoms to which they are attached to form a ring -A-” in relation with a moiety of the structure:

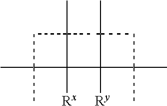

means that R^{x} and R^{y} form the following structure:

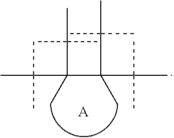

It is also understood that the phrase “—R¹ and an adjacent —R² form a carbon-carbon double bond provided that n is selected from the group consisting of 1, 2, 3 and 4” in relation with a moiety of the structure:

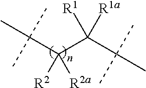

means that for example when n is 1, —R¹ and the adjacent —R² form the following structure:

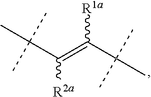

and if for example, n is 2, R¹ and the adjacent —R² form the following structure:

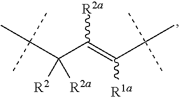

wherein the wavy bond means that —R^{1a} and —R^{2a} may be either on the same side of the double bond, i.e. in cis configuration, or on opposite sides of the double bond, i.e. in trans configuration and wherein the term “adjacent” means that —R¹ and —R² are attached to carbon atoms that are next to each other.

It is also understood that the phrase “two adjacent —R² form a carbon-carbon double bond provided that n is selected from the group consisting of 2, 3 and 4” in relation with a moiety of the structure:

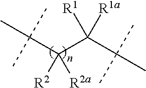

means that for example when n is 2, two adjacent —R² form the following structure:

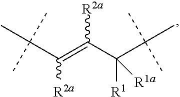

wherein the wavy bond means that each —R^{2a} may be either on the same side of the double bond, i.e. in cis configuration, or on opposite sides of the double bond, i.e. in trans configuration and wherein the term “adjacent” means that two —R² are attached to carbon atoms that are next to each other.

As used herein, the term “excipient” refers to a diluent, adjuvant or vehicle with which the therapeutic, such as a drug or conjugate, is administered. Such pharmaceutical excipient can be sterile liquids, such as water and oils, including those of petroleum, animal, vegetable or synthetic origin, including but not limited to peanut oil, soybean oil, mineral oil, sesame oil and the like. Water is a preferred excipient when the pharmaceutical composition is administered orally. Saline and aqueous dextrose are preferred excipients when the pharmaceutical composition is administered intravenously. Saline solutions and aqueous dextrose and glycerol solutions are preferably employed as liquid excipients for injectable solutions. Suitable pharmaceutical excipients include starch, glucose, lactose, sucrose, mannitol, trehalose, gelatin, malt, rice, flour, chalk, silica gel, sodium stearate, glycerol monostearate, talc, sodium chloride, dried skim milk, glycerol, propylene, glycol, hyaluronic acid, propylene glycol, water, ethanol and the like. The pharmaceutical composition, if desired, can also contain minor amounts of wetting or emulsifying agents, pH buffering agents, like, for example, acetate, succinate, tris, carbonate, phosphate, HEPES (4-(2-hydroxyethyl)-1-piperazineethanesulfonic acid), MES (2-(N-morpholino)ethanesulfonic acid) or can contain detergents, like Tween®, poloxamers, poloxamines, CHAPS, Igepal® or amino acids like, for example, glycine, lysine or histidine. These pharmaceutical compositions can take the form of solutions, suspensions, emulsions, tablets, pills, capsules, powders, sustained-release formulations and the like. The pharmaceutical composition can be formulated as a suppository, with traditional binders and excipients such as triglycerides. Oral formulation can include standard excipients such as pharmaceutical grades of mannitol, lactose, starch, magnesium stearate, sodium saccharine, cellulose, magnesium carbonate, etc. Such compositions will contain a therapeutically effective amount of the drug or drug moiety, together with a suitable amount of excipient so as to provide the form for proper administration to the patient. The formulation should suit the mode of administration.

As used herein, the term “free form” of a drug refers to the drug in its unmodified, pharmacologically fully active form, e.g. after being released from the conjugate.

As used herein, the term “functional group” means a group of atoms which can react with other groups of atoms. Exemplary functional groups are carboxylic acid, primary amine, secondary amine, tertiary amine, maleimide, thiol, sulfonic acid, carbonate, carbamate, hydroxyl, aldehyde, ketone, hydrazine, isocyanate, isothiocyanate, phosphoric acid, phosphonic acid, haloacetyl, alkyl halide, acryloyl, aryl fluoride, hydroxylamine, disulfide, sulfonamides, sulfuric acid, vinyl sulfone, vinyl ketone, diazoalkane, oxirane and aziridine.

As used herein, the term “halogen” means fluoro, chloro, bromo or iodo. In certain embodiments, halogen is fluoro or chloro.

As used herein, the term “interrupted” means that a moiety is inserted in between two carbon atoms or—if the insertion is at one of the moiety's ends—between a carbon or heteroatom and a hydrogen atom, in certain embodiments between a carbon and a hydrogen atom.

In case the conjugates of the present invention comprise one or more acidic or basic groups, the invention also comprises their corresponding pharmaceutically or toxicologically acceptable salts, in particular their pharmaceutically utilizable salts. Thus, the conjugates of the present invention comprising acidic groups can be used according to the invention, for example, as alkali metal salts, alkaline earth metal salts or as ammonium salts. More precise examples of such salts include sodium salts, potassium salts, calcium salts, magnesium salts or salts with ammonia or organic amines such as, for example, ethylamine, ethanolamine, triethanolamine or amino acids, or quaternary ammoniums, such as tetrabutylammonium and cetyl trimethylammonium. Conjugates of the present invention comprising one or more basic groups, i.e. groups which can be protonated, can be present and can be used according to the invention in the form of their addition salts with inorganic or organic acids. Examples for suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, trifluoroacetic acid and other acids known to the person skilled in the art. For the person skilled in the art further methods are known for converting the basic group into a cation like the alkylation of an amine group resulting in a positively-charged ammonium group and an appropriate counterion of the salt. If the conjugates of the present invention simultaneously comprise acidic and basic groups, the invention also includes, in addition to the salt forms mentioned, inner salts or betaines (zwitterions). The respective salts can be obtained by customary methods, which are known to the person skilled in the art like, for example by contacting these prodrugs with an organic or inorganic acid or base in a solvent or dispersant, or by anion exchange or cation exchange with other salts. The present invention also includes all salts of the conjugates of the present invention which, owing to low physiological compatibility, are not directly suitable for use in pharmaceuticals but which can be used, for example, as intermediates for chemical reactions or for the preparation of pharmaceutically acceptable salts.

As used herein, the term “pharmaceutically acceptable” means a substance that does not cause harm when administered to a patient and preferably means approved by a regulatory agency, such as the EMA (Europe) and/or the FDA (US) and/or any other national regulatory agency for use in animals, preferably for use in humans.

As used herein, the term “peptide” as used herein refers to a chain of at least 2 and up to and including 50 amino acid monomer moieties, which may also be referred to as “amino acid residues”, linked by peptide (amide) linkages. The amino acid monomers may be selected from the group consisting of proteinogenic amino acids and non-proteinogenic amino acids and may be D- or L-amino acids. The term “peptide” also includes peptidomimetics, such as peptoids, beta-peptides, cyclic peptides and depsipeptides and covers such peptidomimetic chains with up to and including 50 monomer moieties.

As used herein, the term “protein” refers to a chain of more than 50 amino acid monomer moieties, which may also be referred to as “amino acid residues”, linked by peptide linkages, in which preferably no more than 12000 amino acid monomers are linked by peptide linkages, such as no more than 10000 amino acid monomer moieties, no more than 8000 amino acid monomer moieties, no more than 5000 amino acid monomer moieties or no more than 2000 amino acid monomer moieties.

As used herein, the term “small molecule drug” refers to drugs that are organic compounds with a molecular weight of less than 1000 Da, such as less than 900 Da or less than 800 Da. It is understood that nucleobase-based drug moieties, such as adenine or guanine analogues, may also be a type of small molecule drugs.

As used herein, the term “medium molecule drug” refers to drugs that are organic compounds which are not peptides and which are not proteins, and have a molecular weight ranging from and including 1 kDa to 7.5 kDa.

As used herein, the term “polymer” means a molecule comprising repeating structural units, i.e. the monomers, connected by chemical bonds in a linear, circular, branched, crosslinked or dendrimeric way or a combination thereof, which may be of synthetic or biological origin or a combination of both. The monomers may be identical, in which case the polymer is a homopolymer, or may be different, in which case the polymer is a heteropolymer. A heteropolymer may also be referred to as a “copolymer” and includes for example alternating copolymers in which monomers of different types alternate; periodic copolymers in which monomers of different types of monomers are arranged in a repeating sequence; statistical copolymers in which monomers of different types are arranged randomly; block copolymers in which blocks of different homopolymers consisting of only one type of monomers are linked by a covalent bond; and gradient copolymers in which the composition of different monomers changes gradually along a polymer chain. It is understood that a polymer may also comprise one or more other moieties, such as, for example, one or more functional groups. Likewise, it is understood that also a peptide or protein is a polymer, even though the side chains of individual amino acid residues may be different. It is understood that for covalently crosslinked polymers, such as hydrogels, no meaningful molecular weight ranges can be provided.

As used herein, the term “polymeric” or “polymeric moiety” refers to a reagent or a moiety comprising one or more polymers or polymer moieties. A polymeric reagent or moiety may optionally also comprise one or more other moiety/moieties, which in certain embodiments are selected from the group consisting of
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl and tetralinyl;
branching points, such as —CR<, >C< or —N<; and
linkages selected from the group comprising

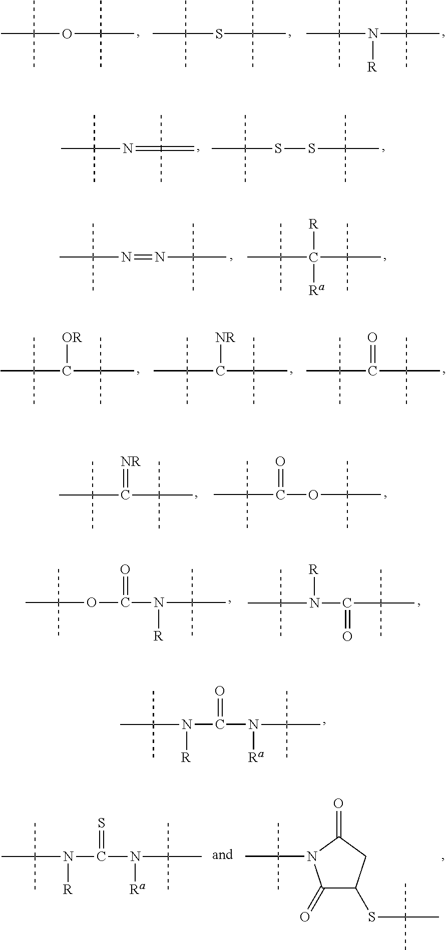

wherein
dashed lines indicate attachment to the remainder of the moiety or reagent;
—R and —R^{a} are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and which moieties and linkages are optionally further substituted.

The person skilled in the art understands that the polymerization products obtained from a polymerization reaction do not all have the same molecular weight, but rather exhibit a molecular weight distribution. Consequently, the molecular weight ranges, molecular weights, ranges of numbers of monomers in a polymer and numbers of monomers in a polymer as used herein, refer to the number average molecular weight and number average of monomers, i.e. to the arithmetic mean of the molecular weight of the polymer or polymeric moiety and the arithmetic mean of the number of monomers of the polymer or polymeric moiety.

Accordingly, in a polymeric moiety comprising “x” monomer units any integer given for “x” therefore corresponds to the arithmetic mean number of monomers. Any range of integers given for “x” provides the range of integers in which the arithmetic mean numbers of monomers lies. An integer for “x” given as “about x” means that the arithmetic mean numbers of monomers lies in a range of integers of x+/−10%, in certain embodiments lies in a range of integers x+/−8%, in certain embodiments lies in a range of integers x+/−5% and in certain embodiments lies in a range of integers x+/−2%.

As used herein, the term “number average molecular weight” means the ordinary arithmetic mean of the molecular weights of the individual polymers.

As used herein, the term “PEG-based” in relation to a moiety or reagent means that said moiety or reagent comprises PEG. In certain embodiments, such PEG-based moiety or reagent comprises at least 10% (w/w) PEG, such as at least 20% (w/w) PEG, such as at least 30% (w/w) PEG, such as at least 40% (w/w) PEG, such as at least 50% (w/w), such as at least 60% (w/w) PEG, such as at least 70% (w/w) PEG, such as at least 80% (w/w) PEG, such as at least 90% (w/w) PEG, such as at least 95% (w/w) PEG. The remaining weight percentage of the PEG-based moiety or reagent may be other moieties, such as those selected from the group consisting of:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl and tetralinyl;
branching points, such as —CR<, >C< or —N<; and
linkages selected from the group comprising

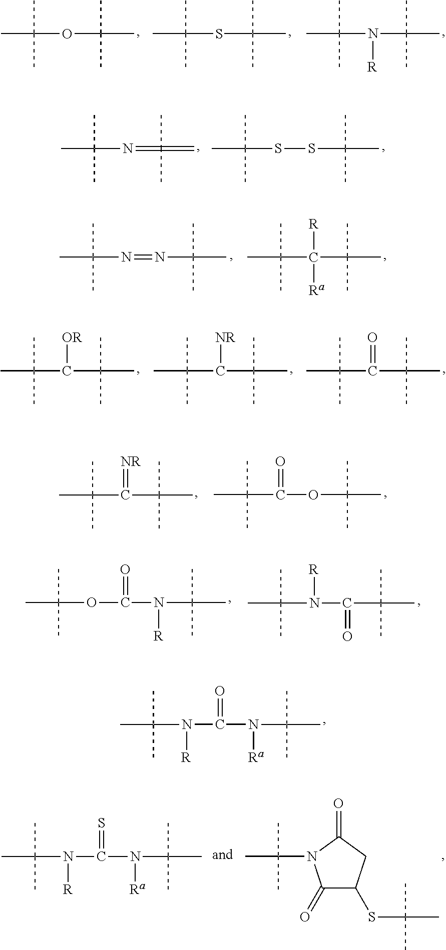

wherein
dashed lines indicate attachment to the remainder of the moiety or reagent;
—R and —R^{a} are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and which moieties and linkages are optionally further substituted.

As used herein, the term “PEG-based comprising at least X % PEG” in relation to a moiety or reagent means that said moiety or reagent comprises at least X % (w/w) ethylene glycol units (—CH₂CH₂O—), wherein the ethylene glycol units may be arranged blockwise, alternating or may be randomly distributed within the moiety or reagent. In certain embodiments, all ethylene glycol units of said moiety or reagent are present in one block; the remaining weight percentage of the PEG-based moiety or reagent are other moieties in certain embodiments selected from the group consisting of:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl;
branching points, such as —CR<, >C< or —N<; and
linkages selected from the group comprising

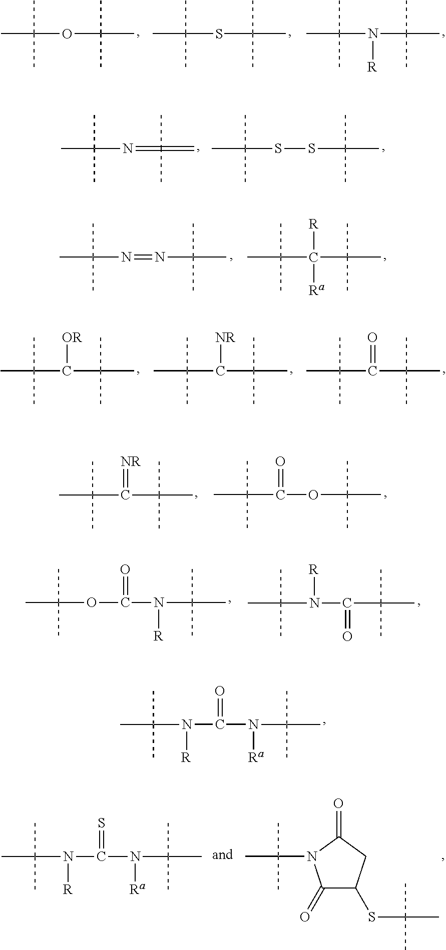

wherein
dashed lines indicate attachment to the remainder of the moiety or reagent, and wherein —R and —R^{a} are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and which moieties and linkages are optionally further substituted.

As used herein, the term “hyaluronic acid-based” in relation to a moiety or reagent means that said moiety or reagent comprises hyaluronic acid. Such hyaluronic acid-based moiety or reagent comprises at least 10% (w/w) hyaluronic acid, such as at least 20% (w/w) hyaluronic acid, such as at least 30% (w/w) hyaluronic acid, such as at least 40% (w/w) hyaluronic acid, such as at least 50% (w/w) hyaluronic acid, such as at least 60% (w/w) hyaluronic acid, such as at least 70% (w/w) hyaluronic acid, such as at least 80% (w/w) hyaluronic acid, such as at least 90% (w/w) hyaluronic acid, or such as at least 95% (w/w) hyaluronic acid. The remaining weight percentage of the hyaluronic acid-based moiety or reagent may be other moieties, such as those selected from the group consisting of
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl;
branching points, such as —CR<, >C< or —N<; and
linkages selected from the group consisting of

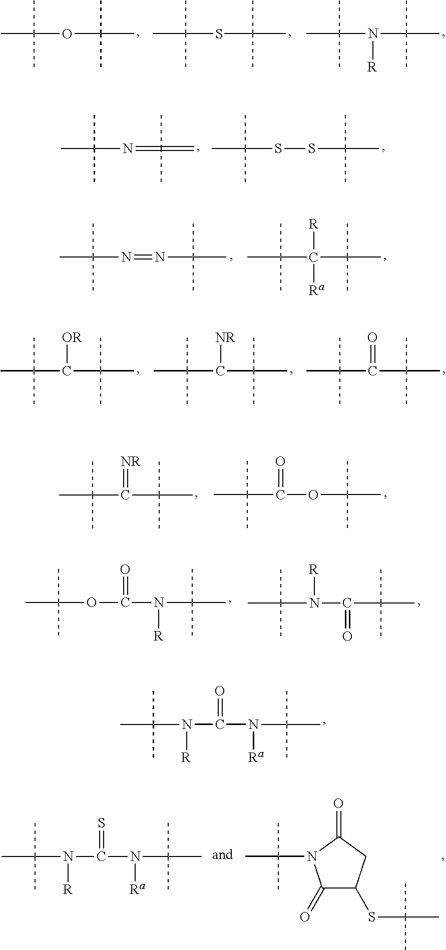

wherein
dashed lines indicate attachment to the remainder of the moiety or reagent;
—R and —R^{a} are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and which moieties and linkages are optionally further substituted.

As used herein, the term “hydrogel” means a hydrophilic or amphiphilic polymeric network composed of homopolymers or copolymers, which is insoluble due to the presence of hydrophobic interactions, hydrogen bonds, ionic interactions and/or covalent chemical crosslinks. The crosslinks provide the network structure and physical integrity.

As used herein, the term “random coil” refers to a peptide or protein adopting/having/forming, in certain embodiments having, a conformation which substantially lacks a defined secondary and tertiary structure as determined by circular dichroism spectroscopy performed in aqueous buffer at ambient temperature, and pH 7.4. In certain embodiments, the ambient temperature is about 20° C., i.e. between 18° C. and 22° C., while in certain embodiments the ambient temperature is 20° C.

As used herein, the term “spacer” or “spacer moiety” refers to a moiety suitable for connecting two moieties. Suitable spacers may be selected from the group consisting of C₁₋₅₀ alkyl, C₂₋₅₀ alkenyl and C₂₋₅₀ alkynyl, which C₁₋₅₀ alkyl, C₂₋₅₀ alkenyl or C₂₋₅₀ alkynyl is optionally interrupted by one or more groups selected from —NH—, —N(C₁₋₄ alkyl)-, —O—, —S—, —C(O)—, —C(O)NH—, —C(O)N(C₁₋₄ alkyl)-, —O—C(O)—, —S(O)—, —S(O)₂—, 4- to 7-membered heterocyclyl, phenyl and naphthyl and may optionally be substituted.

As used herein, the term “substituted” means that one or more —H atom(s) of a molecule or moiety are replaced by a different atom or a group of atoms, which are referred to as “substituent”.

As used herein, the term “substituent” refers in certain embodiments to a moiety selected from the group consisting of halogen, —CN, —C(O)OR^{x1}, —OR^{x1}, —C(O)R^{x1}, —C(O)N(R^{x1})(R^{x1a}), —S(O)₂N(R^{x1})(R^{x1a}), —S(O)N(R^{x1})(R^{x1a}), —S(O)₂R^{x1}, —S(O)R^{x1}, —N(R^{x1})S(O)₂N(R^{x1a})(R^{x1b}), —SR^{x1}, —N(R^{x1})(R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)O^{Rx1a}, —N(R^{x1})C(O)N(R^{x1a})(R^{x1b}), —OC(O)N(R^{x1})(R^{x1a}), -T⁰, C₁₋₅₀ alkyl, C₂₋₅₀ alkenyl and C₂₋₅₀ alkynyl; wherein -T⁰, C₁₋₅₀ alkyl, C₂₋₅₀ alkenyl and C₂₋₅₀ alkynyl are optionally substituted with one or more —R^{x2}, which are the same or different and wherein C₁₋₅₀ alkyl, C₂₋₅₀ alkenyl and C₂₋₅₀ alkynyl are optionally interrupted by one or more groups selected from the group consisting of -T⁰-, —C(O)O—, —O—, —C(O)—, —C(O)N(R^{x3}), —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})— and —OC(O)N(R^{x3})—;

—R^{x1}, —R^{x1a}, —R^{x1b} are independently selected from the group consisting of —H, -T⁰, C₁₋₅₀ alkyl, C₂₋₅₀ alkenyl and C₂₋₅₀ alkynyl; wherein -T⁰, C₁₋₅₀ alkyl, C₂₋₅₀ alkenyl and C₂₋₅₀ alkynyl are optionally substituted with one or more —R^{x2}, which are the same or different and wherein C₁₋₅₀ alkyl, C₂₋₅₀ alkenyl and C₂₋₅₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})— and —OC(O)N(R^{x3})—;
each T⁰ is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl; wherein each T⁰ is independently optionally substituted with one or more —R^{x2} which are the same or different;
each —R^{x2} is independently selected from the group consisting of halogen, —CN, oxo (═O), —C(O)OR^{x4}, —OR^{x4}, —C(O)R^{x4}, —C(O)N(R^{x4})(R^{x4a}), —S(O)₂N(R^{x4})(R^{x4a}), —S(O)N(R^{x4})(R^{x4a}), —S(O)₂R^{x4}, —S(O)R^{x4}, —N(R^{x4})S(O)₂N(R^{x4a})(R^{x4b}), —SR^{x4}, —N(R^{x4})(R^{x4a}), —NO₂, —OC(O)R^{x4}, —N(R^{x4})C(O)R^{x4a}, —N(R^{x4})S(O)₂R^{x4a}, —N(R^{x4})S(O)R^{x4a}, —N(R^{x4})C(O)OR^{x4a}, —N(R^{x4})C(O)N(R^{x4}a)(R^{x4}b), —OC(O)N(R^{x4})(R^{x4}a) and C₁₋₆ alkyl; wherein C₁₋₆ alkyl is optionally substituted with one or more halogen, which are the same or different;
each —R^{x3}, —R^{x3a}, —R^{x4}, —R^{x4a}, —R^{x4b} is independently selected from the group consisting of —H and C₁₋₆ alkyl; wherein C₁₋₆ alkyl is optionally substituted with one or more halogen, which are the same or different.

In certain embodiments, the term “substituent” refers to a moiety selected from the group consisting of halogen, —CN, —COOR^{x1}, —OR^{x1}, —C(O)R^{x1}, —C(O)N(R^{x1})(R^{x1a}), —S(O)₂N(R^{x1})(R^{x1a}), —S(O)N(R^{x1})(R^{x1a}), —S(O)₂R^{x1}, —S(O)R^{x1}, —N(R^{x1})S(O)₂N(R^{x1})(R^{x1a}), —SR^{x1}, —N(R^{x1})(R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1})(R^{x1a}), —OC(O)N(R^{x1})(R^{x1a}), -T⁰, C₁₋₁₀ alkyl, C₂₋₁₀ alkenyl, and C₂₋₁₀ alkynyl; wherein -T⁰, C₁₋₁₀ alkyl, C₂₋₁₀ alkenyl, and C₂₋₁₀ alkynyl are optionally substituted with one or more —R^{x2}, which are the same or different and wherein C₁₋₁₀ alkyl, C₂₋₁₀ alkenyl, and C₂₋₁₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;

each —R^{x1}, —R^{x1a}, —R^{x1b}, —R^{x3}, —R^{x3a} is independently selected from the group consisting of —H, halogen, C₁₋₆ alkyl, C₂₋₆ alkenyl, and C₂₋₆ alkynyl;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R^{x2}, which are the same or different;
each —R^{x2} is independently selected from the group consisting of halogen, —CN, oxo (═O), —C(O)OR^{x4}, —OR^{x4}, —C(O)R^{x4}, —C(O)N(R^{x4})(R^{x4a}), —S(O)₂N(R^{x4})(R^{x4a}), —S(O)N(R^{x4})(R^{x4a}), —S(O)₂R^{x4}, —S(O)R^{x4}, —N(R^{x4})S(O)₂N(R^{x4a})(R^{x4b}), —SR^{x4}, —N(R^{x4})(R^{x4a}), —NO₂, —OC(O)R^{x4}, —N(R^{x4})C(O)R^{x4a}, —N(R^{x4})S(O)₂R^{x4a}, —N(R^{x4})S(O)R^{x4a}, —N(R^{x4})C(O)OR^{x4a}, —N(R^{x4})C(O)N(R^{x4}a)(R^{x4}b), —OC(O)N(R^{x4})(R^{x4}a) and C₁₋₆ alkyl; wherein C₁₋₆ alkyl is optionally substituted with one or more halogen, which are the same or different;
each —R^{x4}, —R^{x4a}, —R^{x4b} is independently selected from the group consisting of —H, halogen, C₁₋₆ alkyl, C₂₋₆ alkenyl, and C₂₋₆ alkynyl.

In certain embodiments, the term “substituent” refers to a moiety selected from the group consisting of halogen, —CN, —COOR^{x1}, —OR^{x1}, —C(O)R^{x1}, —C(O)N(R^{x1})(R^{x1a}), —S(O)₂N(R^{x1})(R^{x1a}), —S(O)N(R^{x1})(R^{x1a}), —S(O)₂R^{x1}, —S(O)R^{x1}, —N(R^{x1})S(O)₂N(R^{x1a})(R^{x1b}), —SR^{x1}, —N(R^{x1})(R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1a})(R^{x1b}), —OC(O)N(R^{x1})(R^{x1a}), -T⁰, C₁₋₆ alkyl, C₂₋₆ alkenyl, and C₂₋₆ alkynyl; wherein -T⁰, C₁₋₆ alkyl, C₂₋₆ alkenyl, and C₂₋₆ alkynyl are optionally substituted with one or more —R^{x2}, which are the same or different and wherein C₁₋₆ alkyl, C₂₋₆ alkenyl, and C₂₋₆ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR³)(R^{x3a})—, —N(R^{x3})C(O)N(R^{x3a})—, and —OC(O)N(R^{x3})—;

each —R^{x1}, —R^{x1a}, —R^{x1b}, —R^{x2}, —R^{x3}, —R^{x3}a is independently selected from the group consisting of —H, halogen, C₁₋₆ alkyl, C₂₋₆ alkenyl, and C₂₋₆ alkynyl;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R^{x}, which are the same or different.

In certain embodiments, a maximum of 6 —H atoms of an optionally substituted molecule are independently replaced by a substituent, e.g. 5 —H atoms are independently replaced by a substituent, 4 —H atoms are independently replaced by a substituent, 3 —H atoms are independently replaced by a substituent, 2 —H atoms are independently replaced by a substituent, or 1 —H atom is replaced by a substituent.

As used herein, the term “therapeutically effective amount” means an amount sufficient to cure, alleviate or partially arrest the clinical manifestations of a given disease and its complications.

Effective amounts for each purpose will depend on the severity of the disease or injury as well as the weight and general state of the subject.

As used herein, the term “water-insoluble” refers to a compound of which less than 1 g can be dissolved in one liter of water at 20° C. to form a homogeneous solution. Accordingly, the term “water-soluble” refers to a compound of which 1 g or more can be dissolved in one liter of water at 20° C. to form a homogeneous solution.

In general, the term “comprise(s)” or “comprising” also encompasses “consist of” or “consisting of”.

It is understood that the “N” in the phrase “π-electron-pair-donating heteroaromatic N” refers to nitrogen.

It is understood that two adjacent —R² in formula (I) can only exist if n is at least 2.

It is understood that the expression “distance between the nitrogen atom marked with an asterisk and the carbon atom marked with an asterisk” refers to the total number of atoms in the shortest distance between the nitrogen and carbon atoms marked with the asterisk and also includes the nitrogen and carbon atoms marked with the asterisk. For example, in the structure below, n is 1 and the distance between the nitrogen marked with an asterisk and the carbon marked with an asterisk is 5:

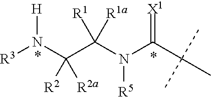

and in the structure below, n is 2, —R¹ and —R^{1a} form a cyclohexyl and the distance between the nitrogen marked with an asterisk and the carbon marked with an asterisk is 6:

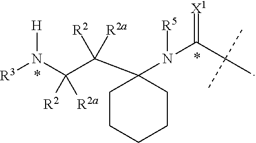

In certain embodiments, all moieties -D of the conjugate are identical, i.e. have the same chemical structure. In such case all moieties -D of the conjugate derive from the same type of drug molecule. It is understood that this means that all moieties -D originate from the same parent drug, but that there may be molecular rearrangements that for example lead to the formation of different tautomeric forms.

In certain embodiments, the conjugate of the present invention comprises different moieties -D, i.e. comprises moieties -D with different chemical structures. These different structures derive from different types of drug molecules. It is understood that this does not include certain molecular rearrangements that for example lead to the formation of different tautomeric forms, which however may also be present. In certain embodiments, the conjugate of the present invention comprises two different types of moieties -D. In certain embodiments, the conjugate of the present invention comprises three different types of moieties -D. In certain embodiments, the conjugate of the present invention comprises four different types of moieties -D. In certain embodiments, the conjugate of the present invention comprises five different types of moieties -D.

If the conjugates of the present invention comprise more than one type of -D, all moieties -D may be conjugated to the same type of -L¹- or may be conjugated to different types of -L¹-, i.e. a first type of -D may be conjugated to a first type of -L¹-, a second type of -D may be conjugated to a second type of -L¹- and so on. Using different types of -L¹- may, in certain embodiments, allow different release kinetics for different types of -D, such as for example a faster release for a first type of -D, a medium release for a second type of -D and a slow release for a third type of -D. Accordingly, in certain embodiments the conjugates of the present invention comprise one type of -L¹-. In certain embodiments, the conjugates of the present invention comprise two types of -L¹-. In certain embodiments, the conjugates of the present invention comprise three types of -L¹-. In certain embodiments, the conjugates of the present invention comprise four types of -L¹-.

In certain embodiments, the conjugates of the present invention comprise one type of -D and one type of -L¹-. In certain embodiments, the conjugates of the present invention comprise two types of -D and two types of -L¹-. In certain embodiments, the conjugates of the present invention comprise three types of -D and three types of -L¹-. In certain embodiments, the conjugates of the present invention comprise four types of -D and four types of -L¹-.

In certain embodiments, all moieties -L¹- of the conjugate have the same structure. In certain embodiments, the conjugate comprises two or more different types of moiety -L¹-, such as for example two, three, four or five different types of moiety -L¹-. Such two or more different types of moiety -L¹- may be conjugated to the same or different type of -D. Using different types of -L¹- allows releasing the same or different type of drug D-H from the conjugate of the present invention with different release half-lives, such as when combining a first group of moieties -L¹- with a short release half-life with a second group of moieties -L¹- with a long release half-life.

In certain embodiments, -D is selected from the group consisting of small molecule, medium size, peptide and protein drug moieties.

In certain embodiments, -D is a small molecule drug moiety. In certain embodiments, such small molecule drug moiety is a nucleobase-based drug moiety.

It is understood that a moiety -D may comprise at least one π-electron-pair-donating heteroaromatic nitrogen atoms, such as for example, one, two, three, four, five, six, seven, eight, nine or ten π-electron-pair-donating heteroaromatic nitrogen atoms. It is also understood that for the peptide and protein drug moieties such nitrogens may be provided by amino acids, such as for example, tryptophan or histidine and for nucleobase-based drug moieties such nitrogens may be provided by adenine or guanine.

In certain embodiments, -D is a peptide drug moiety.

In certain embodiments, -D is a peptide drug moiety selected from the group consisting of C-type natriuretic peptide, parathyroid hormone, W peptide, memno-peptide A and G1 peptide.

In certain embodiments, -D is a protein drug moiety. In certain embodiments, such protein moiety is a monoclonal or polyclonal antibody or fragment or fusion thereof.

In certain embodiments, -D is selected from the group consisting of acitazanolast, seglitide, etodolac, ledazerol, N-desmethylmilameline, carbazomycin G, carbazomycin H, asperlicin C, asperlicin D, desacetylvinblastinehydrazide, jasplakinolide, ageliferin diacetate, ageliferin dihydrochloride, dolasetron, roxindole mesilate, liblomycin, tazanolast, abecamil, verticillatine, liarozole, irtemazole, omeprazole, parodilol hemifumarate, tropisetron, topsentine B1, bromotopsentin, lifarizine, pyrindamycin A, pyrindamycin B, duocarmycin C1, duocarmycin C2, duocarmycin A, biemnidin, elopiprazole, mibefradil, luzindole, manzamine D, manzamine B, manzamine C, octreotide acetate, lazabemide hydrochloride, tetrazolast meglumine, enalkiren, cloturin, pergolide mesylate, liarozole hydrochloride, chloropeptin II, adozelesin, carzelesin, beta-CCM, dexmedetomidine hydrochloride, naratriptan hydrochloride, indanomycin, homoindanomycin, mibefradil hydrochloride, dolasetron mesilate, nicotredole, duocarmycin B1, duocarmycin B2, vincristine sulfate, antiflammin-2, pantoprazole, manzamine A, janthinomycin C, albifylline, janthinomycin A, janthinomycin B, eflumast, voxergolide hydrochloride, gedocamil, temoporfin, proterguride, vinorelbine, cyclo[His-Pro], mepindolol transdermal patch, tubingensin B, methoxatin, mivazerol, atalaphillidine, atalaphillinine, discorhabdin D, lurosetron, naltrindole, azetirelin, bizelesin, intoplicine, cimetidine bismuth citrate, cimetidine bismuth L-tartrate, manzamine F, lecimibide, manzamine E, pyrazoloacridine, duocarmycin SA, vinfosiltine sulfate, nepaprazole, ramorelix, andolast, taltirelin, ramosetron hydrochloride, nafarelin acetate, cipamfylline, romergoline, pazelliptine trihydrochloride monohydrate, pazelliptine trihydrochloride, giracodazole, saviprazole, pibrozelesin hydrochloride, human angiotensin II, ceruletide diethylamine, carvedilol, remikiren mesilate, rolofylline, nortopsentin D, nortopsentin A, nortopsentin B, nortopsentin C, devazepide, atipamezole, imetit, batzelline B, carsatrin, demetomidine, medetomidine, pemetrexed disodium, carvotroline hydrochloride, sumatriptan succinate, alosetron maleate, leminoprazole, atevirdine mesylate, lifarizine hydrochloride, arofylline, nepaprazole, vinleucinol, moxonidine hydrochloride hydrate, lansoprazole, cytoblastin, L-histidinol, montirelin tetrahydrate, fabesetron hydrochloride, O6-benzylguanine, indisetron hydrochloride, pyrrolosporin A, antagonist-G, azatoxin, alpha-methyltryptophan, ecteinascidin 722, ecteinascidin 736, eptifibatide, dexpemedolac, kistamicin A, ilomastat, histrelin acetate, verongamine, spinorphin, delavirdine mesilate, epocarbazolin A, epocarbazolin B, ilatreotide, peldesine, prezatide copper acetate, plevitrexed, carquinostatin A, gavestinel sodium, thiazohalostatin, glycothiohexide alpha, cystamidin A, ciprokiren, immepip, immepyr, fipamezole hydrochloride, rizatriptan sulfate, clobenpropit, nornicotine, cabergoline, porfimer sodium, tezampanel, tenatoprazole, almotriptan, iodoproxyfan, pralmorelin, frovatriptan, pranazepide, rizatriptan benzoate, lomeguatrib, ¹¹¹In-pentetreotide, polydiscamide A, pimobendan, impentamine, apaxifylline, makaluvamine C, makaluvamine D, makaluvamine F, vinflunine, examorelin, pumosetrag hydrochloride, pranlukast hydrate, vilazodone hydrochloride, lanepitant, terguride, avitriptan, cimetidine, naxifylline, buserelin acetate, bopindolol, mepindolol sulfate, carprofen, leuprorelin acetate, oxypertine, elliptinium acetate, indoramin hydrochloride, reserpine, ergotamine tartrate, lisuride maleate, ilaprazole, chondramide A, chondramide B, chondramide C, chondramide D, lavanduquinocin, eletriptan, midaxifylline, indisulam, conivaptan hydrochloride, improgan, edotecarin, dexketoprofen imidazole salt, styloguanidine, ciproxifan, loloatin B, trifluproxim, nemifitide ditriflutate, beta-methyl-6-chloromelatonin, argyrin B, argyrin A, 18-hydroxycoronaridine, 18-methoxycoronaridine, fadolmidine hydrochloride, semaxanib, kurasoin B, avorelin, gilvusmycin, tegaserod maleate, carbazomadurin A, carbazomadurin B, rafabegron, nepadutant, donitriptan mesilate, becatecarin, donitriptan hydrochloride, Yttrium-90 edotreotide, methylhistaprodifen, histaprodifen, lemuteporfin, afeletecan hydrochloride, cipralisant, demethylasterriquinone B-1, indole-3-propionic acid, shermilamine D, decatromicin A, decatromicin B, venorphin, milbemycin alpha-9, alsterpaullone, secobatzelline B, arcyriacyanin A, O-demethylmurrayafoline A, clausenamine A, Secobatzelline A, sabiporide mesilate, alosetron hydrochloride, halimide, imoproxifan, barusiban, calothrixin A, golotimod, tadalafil, fluoroindolocarbazole C, fluoroindolocarbazole A, fluoroindolocarbazole B, denibulin hydrochloride, 99mTc-c(RGDfK*)2HYNIC, sunitinib, sunitinib malate, indolmycin, 2,7-dibromocryptolepine, pasireotide, calindol dihydrochloride, dacinostat, gilatide, pyridone-6, forodesine hydrochloride, sotrastaurin, gastrazole, yatakemycin, antileukinate, dovitinib lactate, axitinib, pruvanserin hydrochloride, shishijimicin C, shishijimicin A, shishijimicin B, plinabulin, DADMe-immucillin-G, DADMe-immucillin-H, cediranib, bremelanotide, immethridine, talaporfin sodium, methanobactin, [D-Tyr1] MS-10, [Arg(Me)9] MS-10, [D-Tyr1,Arg(Me)9] MS-10, [Trp19] MS-10, [D-Tyr1,AzaGly7,Arg(Me)9] MS-10, bederocin, methimepip, pachymedusa dacnicolor tryptophyllin-1, obatoclax mesylate, necrostatin-1, 2-bromo-7-nitrocryptolepine, 7-bromo-2-chlorocryptolepine, brivanib alaninate, brivanib, danusertib, afobazole, centanamycin, linifanib, ethylthio-DADMe-immucillin-A, 4-chlorophenylthio-DADMe-immucillin-A, methylthio-DADMe-immucillin-A, radezolid, shepherdin, desacetylvinblastinehydrazide folate conjugate, anamorelin hydrochloride, histrelin, mercaptopurine, histamine dihydrochloride, bleomycin A2 sulfate, bromocriptine mesilate, timodepressin, yohimbine, peplomycin, detomidine hydrochloride, vindesine, desglugastrin tromethamine, dihydroergotamine mesylate, oglufanide disodium, cefpimizole sodium, tinazoline hydrohloride, panobinostat, lanreotide acetate, pindolol, kinetin, reversine, carteramine A, meriolin-3, pymeprazole, 3-indole, PPI17-24, dexlansoprazole, lecirelin, methylhomoindanomycin, deslorelin, fabesetron, carmoxirole hydrochloride, galdansetron, melanotan II, nocathiacin II, theophylline, turofexorate isopropyl, marinopyrrole A, amycolamicin, calpinactam, microbisporicin A2, beta-amyloid (12-20), 5-fluorouracil, thioguanine, pemetrexed, mercaptopurine, tivantinib, ulixertinib, MK-8353, SCH772984, idelalisib, vemurafenib, EOS-200271 and X4P-001.

In certain embodiments, -D is axitinib.

In certain embodiments, ═X¹ is ═O. In certain embodiments, ═X¹ is ═S. In certain embodiments, ═X¹ is ═N(R⁴).

In certain embodiments, —X²— is —O—. In certain embodiments, —X²— is —S—. In certain embodiments, —X²— is —N(R⁵)—. In certain embodiments, —X²— is —C(R⁶)(R^{6a})—.

In certain embodiments, —X³— is

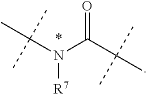

In certain embodiments, —X³— is

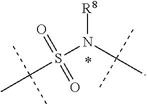

In certain embodiments, —X³— is R

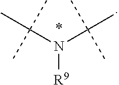

In certain embodiments, —X³— is —C(R¹⁰)(R^{10a})—. In certain embodiments, —X³— is —C(R¹¹)(R^{11a})—C(R¹²)(R^{12a})—. In certain embodiments, —X³— is —O—. In certain embodiments, —X³— is —C(O)—.

In certain embodiments, —X²— is —N(R⁵)—, —X³— is

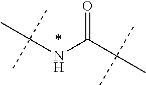

and the distance between the nitrogen atom marked with an asterisk and the carbon atom marked with an asterisk in formula (I) is 5 atoms.

In certain embodiments, —X² is —N(R⁵)—, —X³— is

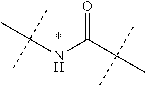

and the distance between the nitrogen atom marked with an asterisk and the carbon atom marked with an asterisk in formula (I) is 6 atoms.

In certain embodiments, —X²— is —N(R⁵)—, —X³— is

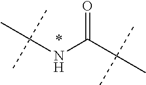

and the distance between the nitrogen atom marked with an asterisk and the carbon atom marked with an asterisk in formula (I) is 7 atoms.

In certain embodiments, —X²— is —N(R⁵)—, —X³— is

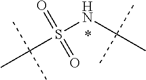

and the distance between the nitrogen atom marked with an asterisk and the carbon atom marked with an asterisk in formula (I) is 5 atoms.

In certain embodiments, —X²— is —N(R⁵)—, —X³— is

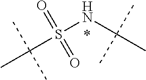

and the distance between the nitrogen atom marked with an asterisk and the carbon atom marked with an asterisk in formula (I) is 6 atoms.

In certain embodiments, —X²— is —N(R⁵)—, —X³— is

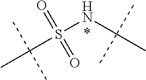

and the distance between the nitrogen atom marked with an asterisk and the carbon atom marked with an asterisk in formula (I) is 7 atoms.

In certain embodiments, —X²— is —N(R⁵)—, —X³— is

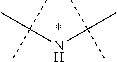

and the distance between the nitrogen atom marked with an asterisk and the carbon atom marked with an asterisk in formula (I) is 5 atoms.

In certain embodiments, —X²— is —N(R⁵)—, —X³— is

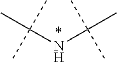

and the distance between the nitrogen atom marked with an asterisk and the carbon atom marked with an asterisk in formula (I) is 6 atoms.

In certain embodiments, —X²— is —N(R⁵)—, —X³— is

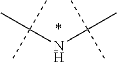

and the distance between the nitrogen atom marked with an asterisk and the carbon atom marked with an asterisk in formula (I) is 7 atoms.

In certain embodiments, —X²— is —N(R⁵)—, —X³— is

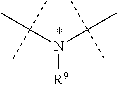

and the distance between the nitrogen atom marked with an asterisk and the carbon atom marked with an asterisk in formula (I) is 5 atoms.

In certain embodiments, —X²— is —N(R⁵)—, —X³— is

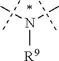

and the distance between the nitrogen atom marked with an asterisk and the carbon atom marked with an asterisk in formula (I) is 6 atoms.

In certain embodiments, —X²— is —N(R⁵)—, —X³— is

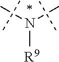

and the distance between the nitrogen atom marked with an asterisk and the carbon atom marked with an asterisk in formula (I) is 7 atoms.

In certain embodiments, ═X¹ is ═O, —X²— is —C(R⁶)(R^{6a})—, —X³— is

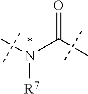

and —R³ does not comprise an amine.

In certain embodiments, —R¹, —R^{1a}, —R⁶, —R^{6a}, —R¹⁰, —R^{10a}, —R¹¹, —R^{11a}, —R¹², —R^{12a} and each of —R² and —R^{2a} are independently selected from the group consisting of —H, —C(O)OH, halogen, —CN, —OH, C₁₋₆ alkyl, C₂₋₆ alkenyl and C₂₋₆ alkynyl.

In certain embodiments, —R¹ is selected from the group consisting of —H, —C(O)OH, halogen, —CN, —OH, C₁₋₆ alkyl, C₂₋₆ alkenyl and C₂₋₆ alkynyl. In certain embodiments, —R¹ is selected from the group consisting of —H, —C(O)OH, —CN, —OH, C₁₋₆ alkyl, C₂₋₆ alkenyl and C₂₋₆ alkynyl. In certain embodiments, —R¹ is selected from the group consisting of —H, —C(O)OH, halogen, —OH, C₁₋₆ alkyl, C₂₋₆ alkenyl and C₂₋₆ alkynyl. In certain embodiments, —R¹ is selected from the group consisting of —H, —C(O)OH, —OH and C₁₋₆ alkyl. In certain embodiments, —R¹ is —H. In certain embodiments, —R¹ is —C(O)OH. In certain embodiments, —R¹ is halogen. In certain embodiments, —R¹ is —F. In certain embodiments, —R¹ is —CN. In certain embodiments, —R¹ is —OH. In certain embodiments, —R¹ is C₁₋₆ alkyl. In certain embodiments, —R¹ is C₂₋₆ alkenyl. In certain embodiments, —R¹ is C₂₋₆ alkynyl.

In certain embodiments, —R¹ is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl. In this case it is understood that —R¹/—R^{1a} may optionally be joined together with the atom to which they are attached to form a C₃₋₁₀ cycloalkyl and that one or more of the pairs —R¹/—R², —R¹/—R⁵, —R¹/—R⁶, —R¹/—R⁹ and —R¹/—R¹⁰ may optionally be joined together with the atoms to which they are attached to form a ring -A-, wherein -A- is used as defined for formula (I).

In certain embodiments, —R^{1a} is selected from the group consisting of —H, —C(O)OH, halogen, —CN, —OH, C₁₋₆ alkyl, C₂₋₆ alkenyl and C₂₋₆ alkynyl. In certain embodiments, —R^{1a} is selected from the group consisting of —H, —C(O)OH, —CN, —OH, C₁₋₆ alkyl, C₂₋₆ alkenyl and C₂₋₆ alkynyl. In certain embodiments, —R^{1a} is selected from the group consisting of —H, —C(O)OH, halogen, —OH, C₁₋₆ alkyl, C₂₋₆ alkenyl and C₂₋₆ alkynyl. In certain embodiments, —R^{1a} is selected from the group consisting of —H, —C(O)OH, —OH and C₁₋₆ alkyl. In certain embodiments, —R^{1a} is —H. In certain embodiments, —R^{1a} is —C(O)OH. In certain embodiments, —R^{1a} is halogen. In certain embodiments, —R^{1a} is —F. In certain embodiments, —R^{1a} is —CN. In certain embodiments, —R^{1a} is —OH. In certain embodiments, —R^{1a} is C₁₋₆ alkyl. In certain embodiments, —R^{1a} is C₂₋₆ alkenyl. In certain embodiments, —R^{1a} is C₂₋₆ alkynyl. In certain embodiments, —R^{1a} is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R⁶ is selected from the group consisting of —H, —C(O)OH, halogen, —CN, —OH, C₁₋₆ alkyl, C₂₋₆ alkenyl and C₂₋₆ alkynyl. In certain embodiments, —R⁶ is selected from the group consisting of —H, —C(O)OH, —CN, —OH, C₁₋₆ alkyl, C₂₋₆ alkenyl and C₂₋₆ alkynyl. In certain embodiments, —R⁶ is selected from the group consisting of —H, —C(O)OH, halogen, —OH, C₁₋₆ alkyl, C₂₋₆ alkenyl and C₂₋₆ alkynyl. In certain embodiments, —R⁶ is selected from the group consisting of —H, —C(O)OH, —OH and C₁₋₆ alkyl. In certain embodiments, —R⁶ is —H. In certain embodiments, —R⁶ is —C(O)OH. In certain embodiments, —R⁶ is halogen. In certain embodiments, —R⁶ is —F. In certain embodiments, —R⁶ is —CN. In certain embodiments, —R⁶ is —OH. In certain embodiments, —R⁶ is C₁₋₆ alkyl. In certain embodiments, —R⁶ is C₂₋₆ alkenyl. In certain embodiments, —R⁶ is C₂₋₆ alkynyl. In certain embodiments, —R⁶ is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R^{6a} is selected from the group consisting of —H, —C(O)OH, halogen, —CN, —OH, C₁₋₆ alkyl, C₂₋₆ alkenyl and C₂₋₆ alkynyl. In certain embodiments, —R^{6a} is selected from the group consisting of —H, —C(O)OH, —CN, —OH, C₁₋₆ alkyl, C₂₋₆ alkenyl and C₂₋₆ alkynyl.

In certain embodiments, —R^{6a} is selected from the group consisting of —H, —C(O)OH, halogen, —OH, C₁₋₆ alkyl, C₂₋₆ alkenyl and C₂₋₆ alkynyl. In certain embodiments, —R^{6a} is selected from the group consisting of —H, —C(O)OH, —OH and C₁₋₆ alkyl. In certain embodiments, —R^{6a} is —H. In certain embodiments, —R^{6a} is —C(O)OH. In certain embodiments, —R^{6a} is halogen. In certain embodiments, —R^{6a} is —F. In certain embodiments, —R^{6a} is —CN. In certain embodiments, —R^{6a} is —OH. In certain embodiments, —R^{6a} is C₁₋₆ alkyl. In certain embodiments, —R^{6a} is C₂₋₆ alkenyl. In certain embodiments, —R^{6a} is C₂₋₆ alkynyl. In certain embodiments, —R^{6a} is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R¹⁰ is selected from the group consisting of —H, —C(O)OH, halogen, —CN, —OH, C₁₋₆ alkyl, C₂₋₆ alkenyl and C₂₋₆ alkynyl. In certain embodiments, —R¹⁰ is selected from the group consisting of —H, —C(O)OH, —CN, —OH, C₁₋₆ alkyl, C₂₋₆ alkenyl and C₂₋₆ alkynyl. In certain embodiments, —R¹⁰ is selected from the group consisting of —H, —C(O)OH, halogen, —OH, C₁₋₆ alkyl, C₂₋₆ alkenyl and C₂₋₆ alkynyl. In certain embodiments, —R¹⁰ is selected from the group consisting of —H, —C(O)OH, —OH and C₁₋₆ alkyl. In certain embodiments, —R¹⁰ is —H. In certain embodiments, —R¹⁰ is —C(O)OH. In certain embodiments, —R¹⁰ is halogen. In certain embodiments, —R¹⁰ is —F. In certain embodiments, —R¹⁰ is —CN. In certain embodiments, —R¹⁰ is —OH. In certain embodiments, —R¹⁰ is C₁₋₆ alkyl. In certain embodiments, —R¹⁰ is C₂₋₆ alkenyl. In certain embodiments, —R¹⁰ is C₂₋₆ alkynyl. In certain embodiments, —R¹⁰ is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R^{10a} is selected from the group consisting of —H, —C(O)OH, halogen, —CN, —OH, C₁₋₆ alkyl, C₂₋₆ alkenyl and C₂₋₆ alkynyl. In certain embodiments, —R^{10a} is selected from the group consisting of —H, —C(O)OH, —CN, —OH, C₁₋₆ alkyl, C₂₋₆ alkenyl and C₂₋₆ alkynyl. In certain embodiments, —R^{10a} is selected from the group consisting of —H, —C(O)OH, halogen, —OH, C₁₋₆ alkyl, C₂₋₆ alkenyl and C₂₋₆ alkynyl. In certain embodiments, —R^{10a} is selected from the group consisting of —H, —C(O)OH, —OH and C₁₋₆ alkyl. In certain embodiments, —R^{10a} is —H. In certain embodiments, —R^{10a} is —C(O)OH. In certain embodiments, —R^{10a} is halogen. In certain embodiments, —R^{10a} is —F. In certain embodiments, —R^{10a} is —CN. In certain embodiments, —R^{10a} is —OH. In certain embodiments, —R^{10a} is C₁₋₆ alkyl. In certain embodiments, —R^{10a} is C₂₋₆ alkenyl. In certain embodiments, —R^{10a} is C₂₋₆ alkynyl. In certain embodiments, —R^{10a} is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R¹¹ is selected from the group consisting of —H, —C(O)OH, halogen, —CN, —OH, C₁₋₆ alkyl, C₂₋₆ alkenyl and C₂₋₆ alkynyl. In certain embodiments, —R¹¹ is selected from the group consisting of —H, —C(O)OH, —CN, —OH, C₁₋₆ alkyl, C₂₋₆ alkenyl and C₂₋₆ alkynyl.

In certain embodiments, —R¹¹ is selected from the group consisting of —H, —C(O)OH, halogen, —OH, C₁₋₆ alkyl, C₂₋₆ alkenyl and C₂₋₆ alkynyl. In certain embodiments, —R¹¹ is selected from the group consisting of —H, —C(O)OH, —OH and C₁₋₆ alkyl. In certain embodiments, —R¹¹ is —H. In certain embodiments, —R¹¹ is —C(O)OH. In certain embodiments, —R¹¹ is halogen. In certain embodiments, —R¹¹ is —F. In certain embodiments, —R¹¹ is —CN. In certain embodiments, —R¹¹ is —OH. In certain embodiments, —R¹¹ is C₁₋₆ alkyl. In certain embodiments, —R¹¹ is C₂₋₆ alkenyl. In certain embodiments, —R¹¹ is C₂₋₆ alkynyl. In certain embodiments, —R¹¹ is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R^{11a} is selected from the group consisting of —H, —C(O)OH, halogen, —CN, —OH, C₁₋₆ alkyl, C₂₋₆ alkenyl and C₂₋₆ alkynyl. In certain embodiments, —R^{11a} is selected from the group consisting of —H, —C(O)OH, —CN, —OH, C₁₋₆ alkyl, C₂₋₆ alkenyl and C₂₋₆ alkynyl. In certain embodiments, —R^{11a} is selected from the group consisting of —H, —C(O)OH, halogen, —OH, C₁₋₆ alkyl, C₂₋₆ alkenyl and C₂₋₆ alkynyl. In certain embodiments, —R^{11a} is selected from the group consisting of —H, —C(O)OH, —OH and C₁₋₆ alkyl. In certain embodiments, —R^{11a} is —H. In certain embodiments, —R^{11a} is —C(O)OH. In certain embodiments, —R^{11a} is halogen. In certain embodiments, —R^{11a} is —F. In certain embodiments, —R^{11a} is —CN. In certain embodiments, —R^{11a} is —OH. In certain embodiments, —R^{11a} is C₁₋₆ alkyl. In certain embodiments, —R^{11a} is C₂₋₆ alkenyl. In certain embodiments, —R^{11a} is C₂₋₆ alkynyl. In certain embodiments, —R^{11a} is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R¹² is selected from the group consisting of —H, —C(O)OH, halogen, —CN, —OH, C₁₋₆ alkyl, C₂₋₆ alkenyl and C₂₋₆ alkynyl. In certain embodiments, —R¹² is selected from the group consisting of —H, —C(O)OH, —CN, —OH, C₁₋₆ alkyl, C₂₋₆ alkenyl and C₂₋₆ alkynyl. In certain embodiments, —R¹² is selected from the group consisting of —H, —C(O)OH, halogen, —OH, C₁₋₆ alkyl, C₂₋₆ alkenyl and C₂₋₆ alkynyl. In certain embodiments, —R¹² is selected from the group consisting of —H, —C(O)OH, —OH and C₁₋₆ alkyl. In certain embodiments, —R¹² is —H. In certain embodiments, —R¹² is —C(O)OH. In certain embodiments, —R¹² is halogen. In certain embodiments, —R¹² is —F. In certain embodiments, —R¹² is —CN. In certain embodiments, —R¹² is —OH. In certain embodiments, —R¹² is C₁₋₆ alkyl. In certain embodiments, —R¹² is C₂₋₆ alkenyl. In certain embodiments, —R¹² is C₂₋₆ alkynyl. In certain embodiments, —R¹² is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R^{12a} is selected from the group consisting of —H, —C(O)OH, halogen, —CN, —OH, C₁₋₆ alkyl, C₂₋₆ alkenyl and C₂₋₆ alkynyl. In certain embodiments, —R^{12a} is selected from the group consisting of —H, —C(O)OH, —CN, —OH, C₁₋₆ alkyl, C₂₋₆ alkenyl and C₂₋₆ alkynyl. In certain embodiments, —R^{12a} is selected from the group consisting of —H, —C(O)OH, halogen, —OH, C₁₋₆ alkyl, C₂₋₆ alkenyl and C₂₋₆ alkynyl. In certain embodiments, —R^{12a} is selected from the group consisting of —H, —C(O)OH, —OH and C₁₋₆ alkyl. In certain embodiments, —R^{12a} is —H. In certain embodiments, —R^{12a} is —C(O)OH. In certain embodiments, —R^{12a} is halogen. In certain embodiments, —R^{12a} is —F. In certain embodiments, —R^{12a} is —CN. In certain embodiments, —R^{12a} is —OH. In certain embodiments, —R^{12a} is C₁₋₆ alkyl. In certain embodiments, —R^{12a} is C₂₋₆ alkenyl. In certain embodiments, —R^{12a} is C₂₋₆ alkynyl. In certain embodiments, —R^{12a} is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, each of —R² is independently selected from the group consisting of —H, —C(O)OH, halogen, —CN, —OH, C₁₋₆ alkyl, C₂₋₆ alkenyl and C₂₋₆ alkynyl. In certain embodiments, each of —R² is independently selected from the group consisting of —H, —C(O)OH, —CN, —OH, C₁₋₆ alkyl, C₂₋₆ alkenyl and C₂₋₆ alkynyl. In certain embodiments, each of —R² is independently selected from the group consisting of —H, —C(O)OH, halogen, —OH, C₁₋₆ alkyl, C₂₋₆ alkenyl and C₂₋₆ alkynyl. In certain embodiments, each of —R² is independently selected from the group consisting of —H, —C(O)OH, —OH and C₁₋₆ alkyl. In certain embodiments, each of —R² is —H. In certain embodiments, each of —R² is —C(O)OH. In certain embodiments, each of —R² is halogen. In certain embodiments, each of —R² is —F. In certain embodiments, each of —R² is —CN. In certain embodiments, each of —R² is —OH. In certain embodiments, each of —R² is C₁₋₆ alkyl. In certain embodiments, each of —R² is C₂₋₆ alkenyl. In certain embodiments, each of —R² is C₂₋₆ alkynyl.

In certain embodiments, each of —R² is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl. In this case it is understood that one or more of the pairs —R²/—R^{2a} and two adjacent —R² may optionally be joined with the atom to which they are attached to form a C₃₋₁₀ cycloalkyl and that the pair —R²/—R⁵ may optionally be joined together with the atoms to which they are attached to form a ring -A-, wherein -A- is used as defined in formula (I).

In certain embodiments, each of —R^{2a} is independently selected from the group consisting of —H, —C(O)OH, halogen, —CN, —OH, C₁₋₆ alkyl, C₂₋₆ alkenyl and C₂₋₆ alkynyl. In certain embodiments, each of —R^{2a} is independently selected from the group consisting of —H, —C(O)OH, —CN, —OH, C₁₋₆ alkyl, C₂₋₆ alkenyl and C₂₋₆ alkynyl. In certain embodiments, each of —R^{2a} is independently selected from the group consisting of —H, —C(O)OH, halogen, —OH, C₁₋₆ alkyl, C₂₋₆ alkenyl and C₂₋₆ alkynyl. In certain embodiments, each of —R^{2a} is independently selected from the group consisting of —H, —C(O)OH, —OH and C₁₋₆ alkyl. In certain embodiments, each of —R^{2a} is —H. In certain embodiments, each of —R^{2a} is —C(O)OH. In certain embodiments, each of —R^{2a} is halogen. In certain embodiments, each of —R^{2a} is —F. In certain embodiments, each of —R^{2a} is —CN. In certain embodiments, each of —R^{2a} is —OH. In certain embodiments, each of —R^{2a} is C₁₋₆ alkyl. In certain embodiments, each of —R^{2a} is C₂₋₆ alkenyl. In certain embodiments, each of —R^{2a} is C₂₋₆ alkynyl. In certain embodiments, each of —R^{2a} is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R³, —R⁴, —R⁵, —R⁷, —R⁸ and —R⁹ are independently selected from the group consisting of —H, -T, —CN, C₁₋₆ alkyl, C₂₋₆ alkenyl and C₂₋₆ alkynyl. In certain embodiments, —R³, —R⁴, —R⁵, —R⁷, —R⁸ and —R⁹ are independently selected from the group consisting of —H, -T, —CN, C₁₋₆ alkyl and C₂₋₆ alkenyl. In certain embodiments, —R³, —R⁴, —R⁵, —R⁷, —R⁸ and —R⁹ are independently selected from the group consisting of —H, -T, —CN and C₁₋₆ alkyl. In certain embodiments, —R³, —R⁴, —R⁵, —R⁷, —R⁸ and —R⁹ are independently selected from the group consisting of —H, -T and C₁₋₆ alkyl. In certain embodiments, —R³, —R⁴, —R⁵, —R⁷, —R⁸ and —R⁹ are independently selected from the group consisting of —H and C₁₋₆ alkyl.

In certain embodiments, —R³ is selected from the group consisting of —H, -T, —CN, C₁₋₆ alkyl, C₂₋₆ alkenyl and C₂₋₆ alkynyl. In certain embodiments, —R³ is —H. In certain embodiments, —R³ is -T. In certain embodiments, —R³ is —CN. In certain embodiments, —R³ is C₁₋₆ alkyl. In certain embodiments, —R³ is C₂₋₆ alkenyl. In certain embodiments, —R³ is C₂₋₆ alkynyl.

In certain embodiments, —R⁴ is selected from the group consisting of —H, -T, —CN, C₁₋₆ alkyl, C₂₋₆ alkenyl and C₂₋₆ alkynyl. In certain embodiments, —R⁴ is —H. In certain embodiments, —R⁴ is -T. In certain embodiments, —R⁴ is —CN. In certain embodiments, —R⁴ is C₁₋₆ alkyl. In certain embodiments, —R⁴ is C₂₋₆ alkenyl. In certain embodiments, —R⁴ is C₂₋₆ alkynyl.

In certain embodiments, —R⁵ is selected from the group consisting of —H, -T, —CN, C₁₋₆ alkyl, C₂₋₆ alkenyl and C₂₋₆ alkynyl. In certain embodiments, —R⁵ is —H. In certain embodiments, —R⁵ is -T. In certain embodiments, —R⁵ is —CN. In certain embodiments, —R⁵ is C₁₋₆ alkyl. In certain embodiments, —R⁵ is C₂₋₆ alkenyl. In certain embodiments, —R⁵ is C₂₋₆ alkynyl.

In certain embodiments, —R⁷ is selected from the group consisting of —H, -T, —CN, C₁₋₆ alkyl, C₂₋₆ alkenyl and C₂₋₆ alkynyl. In certain embodiments, —R⁷ is —H. In certain embodiments, —R⁷ is -T. In certain embodiments, —R⁷ is —CN. In certain embodiments, —R⁷ is C₁₋₆ alkyl. In certain embodiments, —R⁷ is C₂₋₆ alkenyl. In certain embodiments, —R⁷ is C₂₋₆ alkynyl.

In certain embodiments, —R⁸ is selected from the group consisting of —H, -T, —CN, C₁₋₆ alkyl, C₂₋₆ alkenyl and C₂₋₆ alkynyl. In certain embodiments, —R⁸ is —H. In certain embodiments, —R⁸ is -T. In certain embodiments, —R⁸ is —CN. In certain embodiments, —R⁸ is C₁₋₆ alkyl. In certain embodiments, —R⁸ is C₂₋₆ alkenyl. In certain embodiments, —R⁸ is C₂₋₆ alkynyl.

In certain embodiments, —R⁹ is selected from the group consisting of —H, -T, —CN, C₁₋₆ alkyl, C₂₋₆ alkenyl and C₂₋₆ alkynyl. In certain embodiments, —R⁹ is —H. In certain embodiments, —R⁹ is -T. In certain embodiments, —R⁹ is —CN. In certain embodiments, —R⁹ is C₁₋₆ alkyl. In certain embodiments, —R⁹ is C₂₋₆ alkenyl. In certain embodiments, —R⁹ is C₂₋₆ alkynyl.

In certain embodiments, T is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl. In certain embodiments, T is phenyl. In certain embodiments, T is naphthyl. In certain embodiments, T is indenyl. In certain embodiments, T is indanyl. In certain embodiments, T is tetralinyl. In certain embodiments, T is C₃₋₁₀ cycloalkyl. In certain embodiments, T is 3- to 10-membered heterocyclyl. In certain embodiments, T is 8- to 11-membered heterobicyclyl.

In certain embodiments, T is substituted with one or more —R¹³, which are the same or different. In certain embodiments, T is substituted with one —R¹³. In certain embodiments, T is not substituted with —R¹³.

In certain embodiments, —R¹³ is selected from the group consisting of —H, —NO₂, —OCH₃, —CN, —N(R¹⁴)(R^{14a}), —OH, —C(O)OH and C₁₋₆ alkyl. In certain embodiments, —R¹³ is —H. In certain embodiments, —R¹³ is —NO₂. In certain embodiments, —R¹³ is —OCH₃. In certain embodiments, —R¹³ is —CN. In certain embodiments, —R¹³ is —N(R¹⁴)(R^{14a}). In certain embodiments, —R¹³ is —OH. In certain embodiments, —R¹³ is —C(O)OH. In certain embodiments, —R¹³ is C₁₋₆ alkyl.

In certain embodiments, —R¹⁴ and —R^{14a} are independently selected from the group consisting of —H and C₁₋₆ alkyl. In certain embodiments, —R¹⁴ is —H. In certain embodiments, —R¹⁴ is C₁₋₆ alkyl. In certain embodiments, —R^{14a} is —H. In certain embodiments, —R^{14a} is C₁₋₆ alkyl.

In certain embodiments, —R³/—R⁹ are joined with the nitrogen atom to which they are attached to form a 3- to 10-membered heterocyclyl or an 8- to 11-membered heterobicyclyl. In certain embodiments, —R³/—R⁹ are joined with the nitrogen atom to which they are attached to form a 3- to 10-membered heterocyclyl or an 8- to 11-membered heterobicyclyl, wherein the attachment of the 3- to 10-membered heterocyclyl or 8- to 11-membered heterobicyclyl to the rest of the linker moiety of formula (I) takes place via a sp³-hybridized nitrogen.

In certain embodiments, —R³/—R⁹ are joined with the nitrogen atom to which they are attached to form a ring selected from the group consisting of aziridine, azetidine, pyrroline, imidazoline, pyrazoline, 4-thiazoline, pyrrolidine, imidazolidine, pyrazolidine, oxazolidine, isoxazolidine, thiazolidine, isothiazolidine, thiadiazolidine, piperazine, piperidine, morpholine, triazolidine, tetrazolidine, diazepane, homopiperazine, indoline, benzimidazoline, dihydroquinazoline, dihydroquinoline, tetrahydroquinoline, decahydroquinoline, decahydroisoquinoline, tetrahydroisoquinoline and dihydroisoquinoline. Each hydrogen atom of such rings may be replaced by a substituent as defined above.

In certain embodiments, n is selected from the group consisting of 0, 1, 2 and 3. In certain embodiments, n is selected from the group consisting of 0, 1 and 2. In certain embodiments, n is selected from the group consisting of 0 and 1. In certain embodiments, n is 0. In certain embodiments, n is 1. In certain embodiments, n is 2. In certain embodiments, n is 3. In certain embodiments, n is 4.

In certain embodiments, -L¹- is connected to -D through a linkage selected from the group consisting of amide, carbamate, dithiocarbamate, 0-thiocarbamate, S-thiocarbamate, urea, thiourea, thioamide, amidine and guanidine. It is understood that some of these linkages may not be reversible per se, but that in the present invention neighboring groups present in -L¹-, such as for example amide, primary amine, secondary amine and tertiary amine, render these linkages reversible.

In certain embodiments, -L¹- is conjugated to -D through an amide linkage, i.e. ═X¹ is ═O and —X²— is —C(R⁶)(R^{6a})—.

In certain embodiments, -L¹- is conjugated to -D through a carbamate linkage, i.e. ═X¹ is ═O and —X²— is —O—.

In certain embodiments, -L¹- is conjugated to -D through a dithiocarbamate linkage, i.e. ═X¹ is ═S and —X²— is —S—.

In certain embodiments, -L¹- is conjugated to -D through an O-thiocarbamate linkage, i.e. ═X¹ is ═S and —X²— is —O—.

In certain embodiments, -L¹- is conjugated to -D through a S-thiocarbamate linkage, i.e. ═X¹ is ═O and —X²— is —S—.

In certain embodiments, -L¹- is conjugated to -D through a urea linkage, i.e. ═X¹ is ═O and —X²— is —N(R⁵)—.

In certain embodiments, -L¹- is conjugated to -D through a thiourea linkage, i.e. ═X¹ is ═S and —X²— is —N(R⁵)—.

In certain embodiments, -L¹- is conjugated to -D through a thioamide linkage, i.e. ═X¹ is ═S and —X²— is —C(R⁶)(R^{6a}).

In certain embodiments, -L¹- is conjugated to -D through an amidine linkage, i.e. ═X¹ is ═N(R⁴) and —X²— is —C(R⁶)(R^{6a})—.

In certain embodiments, -L¹- is conjugated to -D through a guanidine linkage, i.e. ═X¹ is ═N(R⁴) and —X²— is —N(R⁵)—.

In certain embodiments, -L¹- is further substituted with one or more substituents.

In certain embodiments, -L¹- is not further substituted.

In certain embodiments, all moieties -L²- of the conjugate of the present invention are identical.

In certain embodiments, the conjugate of the present invention comprises more than one type of -L²-, such as two, three, four or five different moieties -L²-. Such more than one type of -L²- may be connected to only one type of -L¹- or may be connected to more than one type of -L¹-.

In certain embodiments, -L²- is a chemical bond.

In certain embodiments, -L²- is a spacer moiety.

In certain embodiments, -L²- is selected from the group consisting of -T′-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a}), —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl and C₂₋₅₀ alkynyl; wherein -T′—, C₁₋₅₀ alkyl, C₂₋₅₀ alkenyl and C₂₋₅₀ alkynyl are optionally substituted with one or more —R^{y2}, which are the same or different and wherein C₁₋₅₀ alkyl, C₂₋₅₀ alkenyl and C₂₋₅₀ alkynyl are optionally interrupted by one or more groups selected from the group consisting of -T′-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})— and —OC(O)N(R^{y3})—;

—R^{y1} and —R^{y1a} are independently selected from the group consisting of —H, -T′, C₁₋₅₀ alkyl, C₂₋₅₀ alkenyl and C₂₋₅₀ alkynyl; wherein -T′, C₁₋₅₀ alkyl, C₂₋₅₀ alkenyl and C₂₋₅₀ alkynyl are optionally substituted with one or more —R^{y2}, which are the same or different, and wherein C₁₋₅₀ alkyl, C₂₋₅₀ alkenyl and C₂₋₅₀ alkynyl are optionally interrupted by one or more groups selected from the group consisting of -T′-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;
each T′ is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl and 8- to 30-membered heteropolycyclyl; wherein each T′ is independently optionally substituted with one or more —R^{y2}, which are the same or different;
each —R^{y2} is independently selected from the group consisting of halogen, —CN, oxo (═O), —C(O)OR^{y5}, —OR^{y5}, —C(O)R^{y5}, —C(O)N(R^{y5})(R^{y5a}), —S(O)₂N(R⁵)(R^{y5a}), —S(O)N(R^{y5})(R^{y5a}), —S(O)₂R^{y5}, —S(O)R^{y5}, —N(R^{y5})S(O)₂N(R^{y5})(R^{y5a}), —SR^{y5}, —N(R^{y5})(R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5})(R^{y5a}), —OC(O)N(R^{y5})(R^{y5a}), and C₁₋₆ alkyl; wherein C₁₋₆ alkyl is optionally substituted with one or more halogen, which are the same or different; and
each —R^{y3}, —R^{y3a}, —R^{y4}, —R^{y4a}, —R^{y5}, —R^{y5a} and —R^{y5b} is independently selected from the group consisting of —H and C₁₋₆ alkyl; wherein C₁₋₆ alkyl is optionally substituted with one or more halogen, which are the same or different.

In certain embodiments, -L²- is selected from the group consisting of -T′-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T′-, C₁₋₂₀ alkyl, C₂₋₂₀ alkenyl, and C₂₋₂₀ alkynyl are optionally substituted with one or more —R^{y2}, which are the same or different and wherein C₁₋₂₀ alkyl, C₂₋₂₀ alkenyl, and C₂₋₂₀ alkynyl are optionally interrupted by one or more groups selected from the group consisting of -T′-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;

—R^{y1} and —R^{y1a} are independently selected from the group consisting of —H, -T′, C₁₋₁₀ alkyl, C₂₋₁₀ alkenyl, and C₂₋₁₀ alkynyl; wherein -T′, C₁₋₁₀ alkyl, C₂₋₁₀ alkenyl, and C₂₋₁₀ alkynyl are optionally substituted with one or more —R^{y2}, which are the same or different, and wherein C₁₋₁₀ alkyl, C₂₋₁₀ alkenyl, and C₂₋₁₀ alkynyl are optionally interrupted by one or more groups selected from the group consisting of -T′-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;
each T′ is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T′ is independently optionally substituted with one or more —R^{y2}, which are the same or different;
—R^{y2} is selected from the group consisting of halogen, —CN, oxo (═O), —C(O)OR^{y5}, —OR^{y5}, —C(O)R^{y5}, —C(O)N(R^{y5})(R^{y5a}), —S(O)₂N(R^{y5})(R^{y5a}), —S(O)N(R^{y5})(R^{y5a}), —S(O)₂R^{y5}, —S(O)R^{y5}, —N(R^{y5})S(O)₂N(R^{y5a})(R^{y5b}), —SR^{y5}, —N(R^{y5})(R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a})(R^{y5b}), —OC(O)N(R^{y5})(R^{y5a}) and C₁₋₆ alkyl; wherein C₁₋₆ alkyl is optionally substituted with one or more halogen, which are the same or different; and each —R^{y3}, —R^{y3a}, —R^{y4}, —R^{y4a}, —R^{y5}, —R^{y5a} and —R^{y5b} is independently selected from the group consisting of —H and C₁₋₆ alkyl; wherein C₁₋₆ alkyl is optionally substituted with one or more halogen, which are the same or different.

In certain embodiments, -L²- is selected from the group consisting of -T′-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T′-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T′-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})— and —OC(O)N(R^{y3})—;

—R^{y1} and —R^{y1a} are independently selected from the group consisting of —H, -T′, C₁₋₁₀ alkyl, C₂₋₁₀ alkenyl and C₂₋₁₀ alkynyl;
each T′ is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl and 8- to 30-membered heteropolycyclyl;
each —R^{y2} is independently selected from the group consisting of halogen, and C₁₋₆ alkyl; and each —R^{y3}, —R^{y3a}, —R^{y4}, —R^{y4a}, —R^{y5}, —R^{y5a} and —R^{y5b} is independently selected from the group consisting of —H and C₁₋₆ alkyl; wherein C₁₋₆ alkyl is optionally substituted with one or more halogen, which are the same or different.

In certain embodiments, -L²- is a C₁₋₂₀ alkyl chain, which is optionally interrupted by one or more groups independently selected from the group consisting of —O—, -T′- and —C(O)N(R^{y1})—; and which C₁₋₂₀ alkyl chain is optionally substituted with one or more groups independently selected from the group consisting of —OH, -T′ and —C(O)N(R^{y6}R^{y6a}); wherein —R^{y1}, —R^{y6}, —R^{y6a} are independently selected from the group consisting of H and C₁₋₄ alkyl and wherein T′ is selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl and 8- to 30-membered heteropolycyclyl.

In certain embodiments, -L²- has a molecular weight in the range of from 14 g/mol to 750 g/mol.

In certain embodiments, -L²- comprises a moiety selected from the group consisting of:

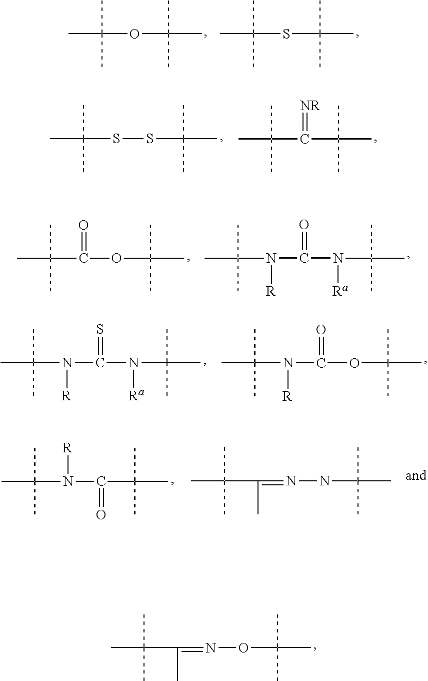

wherein dashed lines indicate attachment to -L¹-, the remainder of -L²- or Z, respectively; and —R and —R^{a} are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

In general, -L²- may be attached to -L¹- at any position where one hydrogen given by —R¹, —R^{1a}, —R², —R^{2a}, —R³, —R⁴, —R⁵, —R⁶, —R^{6a}, —R⁷, —R⁸, —R⁹, —R¹⁰, —R^{10a}, —R¹¹, —R^{11a}, —R¹², —R^{12a}, —R¹³, —R¹⁴ or —R^{14a} is replaced by -L²-.

In certain embodiments, one hydrogen given by —R¹ is replaced by -L²-. In certain embodiments, one hydrogen given by —R^{1a} is replaced by -L²-. In certain embodiments, one hydrogen given by —R² is replaced by -L²-. In certain embodiments, one hydrogen given by —R^{2a} is replaced by -L²-. In certain embodiments, one hydrogen given by —R³ is replaced by -L²-. In certain embodiments, one hydrogen given by —R⁴ is replaced by -L²-. In certain embodiments, one hydrogen given by —R⁵ is replaced by -L²-. In certain embodiments, one hydrogen given by —R⁶ is replaced by -L²-. In certain embodiments, one hydrogen given by —R^{6a} is replaced by -L²-. In certain embodiments, one hydrogen given by —R⁷ is replaced by -L²-. In certain embodiments, one hydrogen given by —R⁸ is replaced by -L²-. In certain embodiments, one hydrogen given by —R⁹ is replaced by -L²-. In certain embodiments, one hydrogen given by —R¹⁰ is replaced by -L²-. In certain embodiments, one hydrogen given by —R^{10a} is replaced by -L²-. In certain embodiments, one hydrogen given by —R¹¹ is replaced by -L²-. In certain embodiments, one hydrogen given by —R^{11a} is replaced by -L²-. In certain embodiments, one hydrogen given by —R¹² is replaced by -L²-. In certain embodiments, one hydrogen given by —R^{12a} is replaced by -L²-. In certain embodiments, one hydrogen given by —R¹³ is replaced by -L²-. In certain embodiments, one hydrogen given by —R¹⁴ is replaced by -L²-. In certain embodiments, one hydrogen given by —R^{14a} is replaced by -L²-.

In certain embodiments, a moiety -L¹-L²- is selected from the group consisting of

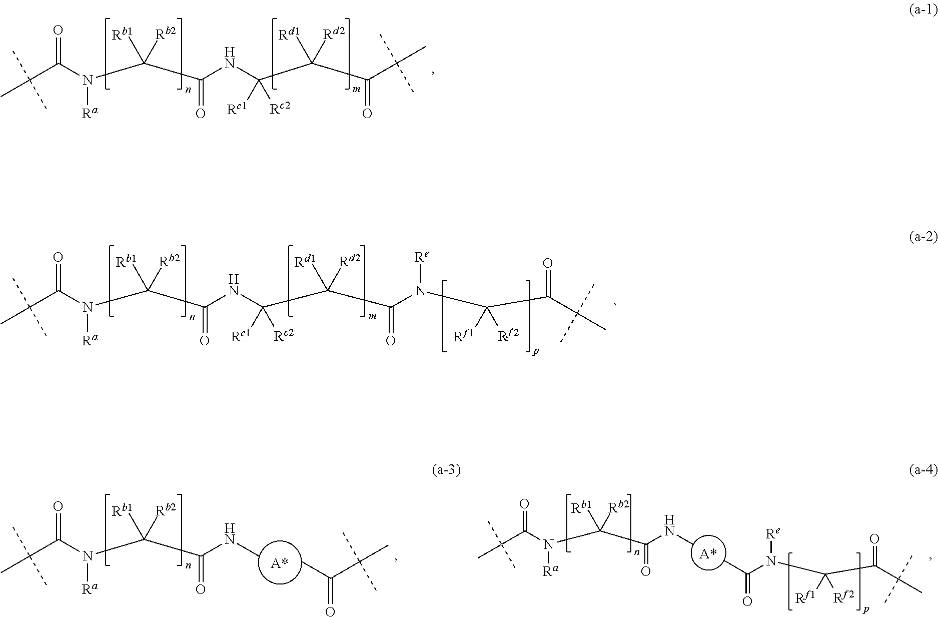

wherein
the dashed line marked with the asterisk indicates attachment to a π-electron-pair-donating heteroaromatic N of -D and the unmarked dashed line indicates attachment to Z, in particular to a nitrogen of an amine of Z;
—R^{a} each —R^{b1}, each —R^{b2}, —R^{c1}, —R^{c2}, each —R^{d1}, each —R^{d2}, —R^{e}, each —R^{f1}, and each —R^{f2} are independently selected from the group consisting of —H and C₁₋₆ alkyl;
n is an integer selected from the group consisting of 1, 2 and 3;
m is an integer selected from the group consisting of 0, 1, 2, 3, 4, 5, 6, 7, 8, 9 and 10;
p is an integer selected from the group consisting of 1, 2, 3, 4, 5, 6, 7, 8, 9, 10, 11, 12, 13, 14, 15, 16, 17, 18, 19 and 20;
A* is a C₃₋₁₀ cycloalkyl; and
optionally, —R^{a} and the adjacent —R^{b1} are joined together with the atoms to which they are attached to form a ring -A-, wherein -A- is used as defined in formula (I).

In certain embodiments, a moiety -L¹-L²- is of formula (a-1). In certain embodiments, —R^{a} of formula (a-1) is selected from the group consisting of —H, methyl and ethyl. In certain embodiments, —R^{a} of formula (a-1) is —H. In certain embodiments, —R^{a} of formula (a-1) is methyl. In certain embodiments, —R^{a} of formula (a-1) is ethyl. In certain embodiments, n of formula (a-1) is selected from the group consisting of 1, 2 and 3. In certain embodiments, n of formula (a-1) is selected from the group consisting of 1 and 2. In certain embodiments, n of formula (a-1) is 1. In certain embodiments, n of formula (a-1) is 2. In certain embodiments, —R^{b1} is selected from the group consisting of —H, methyl and ethyl. In certain embodiments, —R^{b1} of formula (a-1) is —H. In certain embodiments —R^{b1} of formula (a-1) is methyl. In certain embodiments, —R^{b1} of formula (a-1) is ethyl. In certain embodiments, —R^{b2} is selected from the group consisting of —H, methyl and ethyl. In certain embodiments, —R^{b2} of formula (a-1) is —H. In certain embodiments, —R^{b2} of formula (a-1) is methyl. In certain embodiments —R^{b2} of formula (a-1) is ethyl. In certain embodiments, —R^{a} and —R^{b1} of formula (a-1) form a C₅ cycloalkyl. In certain embodiments, n of formula (a-1) is 1 and —R^{a} and —R^{b1} of formula (a-1) form a C₅ cycloalkyl. In certain embodiments, n of formula (a-1) is 1, —R^{a} and —R^{b1} of formula (a-1) form a C₅ cycloalkyl and —R^{b2} is —H. In certain embodiments, —R^{c1} is selected from the group consisting of —H, methyl and ethyl. In certain embodiments, —R^{c1} of formula (a-1) is —H. In certain embodiments, —R¹ of formula (a-1) is methyl. In certain embodiments, —R¹ of formula (a-1) is ethyl. In certain embodiments, —R^{c2} is selected from the group consisting of —H, methyl and ethyl. In certain embodiments, —R^{c2} of formula (a-1) is —H. In certain embodiments, —R^{c2} of formula (a-1) is methyl. In certain embodiments —R^{c2} of formula (a-1) is ethyl. In certain embodiments, —R^{d1} is selected from the group consisting of —H, methyl and ethyl. In certain embodiments, —R^{d1} of formula (a-1) is —H. In certain embodiments —R^{d1} of formula (a-1) is methyl. In certain embodiments, —R^{d1} of formula (a-1) is ethyl. In certain embodiments, —R^{d2} of formula (a-1) is selected from the group consisting of —H, methyl and ethyl. In certain embodiments, —R^{d2} of formula (a-1) is —H. In certain embodiments, —R^{d2} of formula (a-1) is methyl. In certain embodiments, —R^{d2} of formula (a-1) is ethyl. In certain embodiments, m of formula (a-1) is selected from the group consisting of 0, 1, 2, 3, 4, 5 and 6. In certain embodiments, m of formula (a-1) is 0. In certain embodiments, m of formula (a-1) is 1. In certain embodiments, m of formula (a-1) is 2. In certain embodiments, m of formula (a-1) is 4. In certain embodiments, m of formula (a-1) is 5. In certain embodiments, m of formula (a-1) is 6.

In certain embodiments, a moiety -L¹-L²- is of formula (a-2). In certain embodiments, —R^{a} of formula (a-2) is selected from the group consisting of —H, methyl and ethyl. In certain embodiments, —R^{a} of formula (a-2) is —H. In certain embodiments, —R^{a} of formula (a-2) is methyl. In certain embodiments, —R^{a} of formula (a-2) is ethyl. In certain embodiments, n of formula (a-2) is selected from the group consisting of 1, 2 and 3. In certain embodiments, n of formula (a-2) is selected from the group consisting of 1 and 2. In certain embodiments, n of formula (a-2) is 1. In certain embodiments, n of formula (a-2) is 2. In certain embodiments, —R^{b1} is selected from the group consisting of —H, methyl and ethyl. In certain embodiments, —R^{b1} of formula (a-2) is —H. In certain embodiments, —R^{b1} of formula (a-2) is methyl. In certain embodiments, —R^{b1} of formula (a-2) is ethyl. In certain embodiments, —R^{b2} is selected from the group consisting of —H, methyl and ethyl. In certain embodiments, —R^{b2} of formula (a-2) is —H. In certain embodiments, —R^{b2} of formula (a-2) is methyl. In certain embodiments, —R^{b2} of formula (a-2) is ethyl. In certain embodiments, —R^{a} and —R^{b1} of formula (a-2) form a C₅ cycloalkyl. In certain embodiments, n of formula (a-2) is 1 and —R^{a} and —R^{b1} of formula (a-2) form a C₅ cycloalkyl. In certain embodiments, n of formula (a-2) is 1, —R^{a} and —R^{b1} of formula (a-2) form a C₅ cycloalkyl and —R^{b2} is —H. In certain embodiments, —R^{c1} is selected from the group consisting of —H, methyl and ethyl. In certain embodiments, —R^{c1} of formula (a-2) is —H. In certain embodiments, —R¹ of formula (a-2) is methyl. In certain embodiments, —R¹ of formula (a-2) is ethyl. In certain embodiments, —R^{c2} is selected from the group consisting of —H, methyl and ethyl. In certain embodiments, —R^{c2} of formula (a-2) is —H. In certain embodiments, —R^{c2} of formula (a-2) is methyl. In certain embodiments —R^{c2} of formula (a-2) is ethyl. In certain embodiments, —R^{d1} is selected from the group consisting of —H, methyl and ethyl. In certain embodiments, —R^{d1} of formula (a-2) is —H. In certain embodiments —R^{d1} of formula (a-2) is methyl. In certain embodiments, —R^{d1} of formula (a-2) is ethyl. In certain embodiments, —R^{d2} is selected from the group consisting of —H, methyl and ethyl. In certain embodiments, —R^{d2} of formula (a-2) is —H. In certain embodiments, —R^{d2} of formula (a-2) is methyl. In certain embodiments, —R^{d2} of formula (a-2) is ethyl. In certain embodiments, m of formula (a-2) is selected from the group consisting of 0, 1, 2, 3, 4, 5 and 6. In certain embodiments, m of formula (a-2) is 0. In certain embodiments, m of formula (a-2) is 1. In certain embodiments, m of formula (a-2) is 2. In certain embodiments, m of formula (a-2) is 4. In certain embodiments, m of formula (a-2) is 5. In certain embodiments, m of formula (a-2) is 6. In certain embodiments, —R^{e} is selected from the group consisting of —H, methyl and ethyl. In certain embodiments, —R^{e} of formula (a-2) is —H. In certain embodiments, —R^{e} of formula (a-2) is methyl. In certain embodiments, —R^{e} of formula (a-2) is ethyl. In certain embodiments, p of formula (a-2) is selected from the group consisting of 0, 1, 2, 3, 4, 5 and 6. In certain embodiments, p of formula (a-2) is 0. In certain embodiments, p of formula (a-2) is 1. In certain embodiments, p of formula (a-2) is 2. In certain embodiments, p of formula (a-2) is 4. In certain embodiments, p of formula (a-2) is 5. In certain embodiments, p of formula (a-2) is 6. In certain embodiments, —R^{f1} is selected from the group consisting of —H, methyl and ethyl. In certain embodiments, —R¹ of formula (a-2) is —H. In certain embodiments, —R^{f1} of formula (a-2) is methyl. In certain embodiments, —R¹ of formula (a-2) is ethyl. In certain embodiments, —R^{f2} is selected from the group consisting of —H, methyl and ethyl. In certain embodiments, —R^{f2} of formula (a-2) is —H. In certain embodiments, —R^{f} of formula (a-2) is methyl. In certain embodiments, —R² of formula (a-2) is ethyl.

In certain embodiments, a moiety -L¹-L²- is of formula (a-3). In certain embodiments, —R^{a} of formula (a-3) is selected from the group consisting of —H, methyl and ethyl. In certain embodiments, —R^{a} of formula (a-3) is —H. In certain embodiments, —R^{a} of formula (a-3) is methyl.

In certain embodiments, —R^{a} of formula (a-3) is ethyl. In certain embodiments, n of formula (a-3) is selected from the group consisting of 1, 2 and 3. In certain embodiments, n of formula (a-3) is selected from the group consisting of 1 and 2. In certain embodiments, n of formula (a-3) is 1. In certain embodiments, n of formula (a-3) is 2. In certain embodiments, —R^{b1} is selected from the group consisting of —H, methyl and ethyl. In certain embodiments, —R^{b1} of formula (a-3) is —H. In certain embodiments, —R^{b1} of formula (a-3) is methyl. In certain embodiments, —R^{b1} of formula (a-3) is ethyl. In certain embodiments, —R^{b2} is selected from the group consisting of —H, methyl and ethyl. In certain embodiments, —R^{b2} of formula (a-3) is —H. In certain embodiments, —R^{b2} of formula (a-3) is methyl. In certain embodiments, —R^{b2} of formula (a-3) is ethyl. In certain embodiments, —R^{a} and —R^{b1} of formula (a-3) form a C₅ cycloalkyl. In certain embodiments, n of formula (a-3) is 1 and —R^{a} and —R^{b1} of formula (a-3) form a C₅ cycloalkyl. In certain embodiments, n of formula (a-3) is 1, —R^{a} and —R^{b1} of formula (a-3) form a C₅ cycloalkyl and —R^{b2} is —H. In certain embodiments, A* of formula (a-3) is C₅ cycloalkyl. In certain embodiments, A* of formula (a-3) is C₆ cycloalkyl.

In certain embodiments, a moiety -L¹-L²- is of formula (a-4). In certain embodiments, —R^{a} of formula (a-4) is selected from the group consisting of —H, methyl and ethyl. In certain embodiments, —R^{a} of formula (a-4) is —H. In certain embodiments, —R^{a} of formula (a-4) is methyl. In certain embodiments, —R^{a} of formula (a-4) is ethyl. In certain embodiments, n of formula (a-4) is selected from the group consisting of 1, 2 and 3. In certain embodiments, n of formula (a-4) is selected from the group consisting of 1 and 2. In certain embodiments, n of formula (a-4) is 1. In certain embodiments, n of formula (a-4) is 2. In certain embodiments, —R^{b1} is selected from the group consisting of —H, methyl and ethyl. In certain embodiments, —R^{b1} of formula (a-4) is —H. In certain embodiments, —R^{b1} of formula (a-4) is methyl. In certain embodiments —R^{b1} of formula (a-4) is ethyl. In certain embodiments, —R^{b2} is selected from the group consisting of —H, methyl and ethyl. In certain embodiments, —R^{b2} of formula (a-4) is —H. In certain embodiments, —R^{b2} of formula (a-4) is methyl. In certain embodiments, —R^{b2} of formula (a-4) is ethyl. In certain embodiments, —R^{a} and —R^{b1} of formula (a-4) form a C₅ cycloalkyl. In certain embodiments, n of formula (a-4) is 1 and —R^{a} and —R^{b1} of formula (a-4) form a C₅ cycloalkyl. In certain embodiments, n of formula (a-4) is 1, —R^{a} and —R^{b1} of formula (a-4) form a C₅ cycloalkyl and —R^{b2} is —H. In certain embodiments, A* of formula (a-4) is C₅ cycloalkyl. In certain embodiments, A* of formula (a-4) is C₆ cycloalkyl. In certain embodiments, —R^{e} is selected from the group consisting of —H, methyl and ethyl. In certain embodiments, —R^{e} of formula (a-4) is —H. In certain embodiments, —R^{e} of formula (a-4) is methyl. In certain embodiments —R^{e} of formula (a-4) is ethyl. In certain embodiments, p of formula (a-4) is selected from the group consisting of 0, 1, 2, 3, 4, 5 and 6. In certain embodiments, p of formula (a-4) is 0. In certain embodiments, p of formula (a-4) is 1. In certain embodiments, p of formula (a-4) is 2. In certain embodiments, p of formula (a-4) is 4. In certain embodiments, p of formula (a-4) is 5. In certain embodiments, p of formula (a-4) is 6. In certain embodiments, —R^{f1} is selected from the group consisting of —H, methyl and ethyl. In certain embodiments, —R^{f1} of formula (a-4) is —H. In certain embodiments, —R^{f1} of formula (a-4) is methyl. In certain embodiments, —R^{f1} of formula (a-4) is ethyl. In certain embodiments, —R² is selected from the group consisting of —H, methyl and ethyl. In certain embodiments, —R^{f} of formula (a-4) is —H. In certain embodiments, —R^{f} of formula (a-4) is methyl. In certain embodiments, —R² of formula (a-4) is ethyl.

In certain embodiments, a moiety -L¹-L²- is selected from the group consisting of

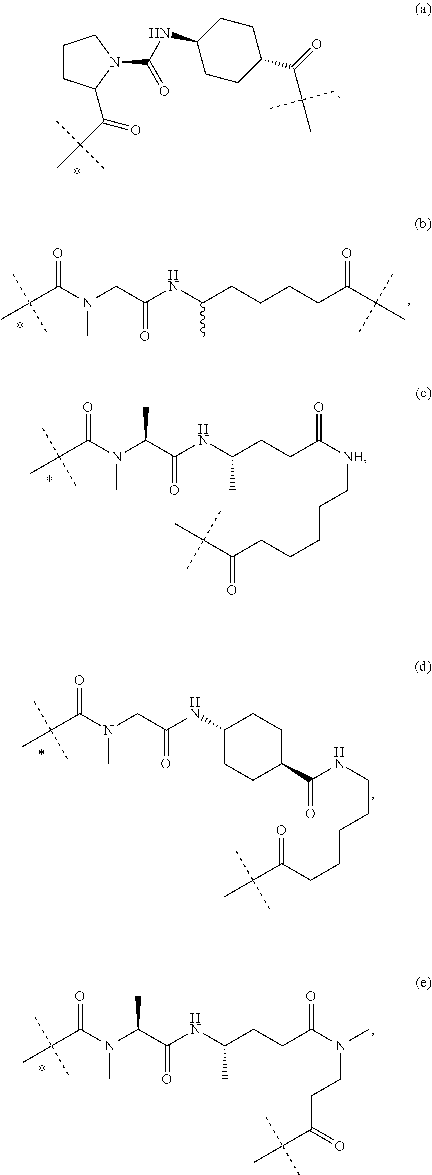

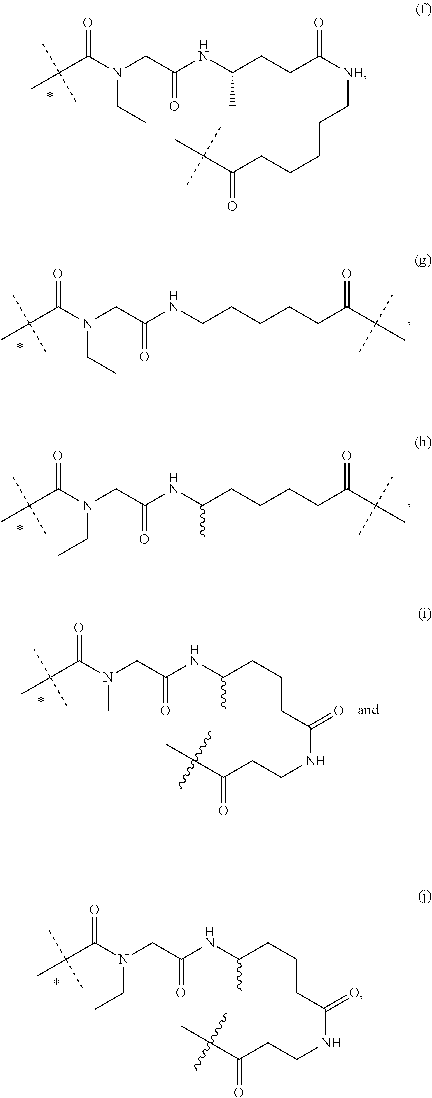

wherein
the dashed line marked with the asterisk indicates attachment to a π-electron-pair-donating heteroaromatic N of -D and the unmarked dashed line indicates attachment to Z, in particular to a nitrogen of an amine of Z.

In certain embodiments, the moiety -L¹-L²- has the structure of formula (a). In certain embodiments, the moiety -L¹-L²- has the structure of formula (b). In certain embodiments, the moiety -L¹-L²- has the structure of formula (c). In certain embodiments, the moiety -L¹-L²- has the structure of formula (d). In certain embodiments, the moiety -L¹-L²- has the structure of formula (e). In certain embodiments, the moiety -L¹-L²- has the structure of formula (f). In certain embodiments, the moiety -L¹-L²- has the structure of formula (g). In certain embodiments, the moiety -L¹-L²- has the structure of formula (h). In certain embodiments, the moiety -L¹-L²- has the structure of formula (i). In certain embodiments, the moiety -L¹-L²- has the structure of formula (j).

In certain embodiments, the dashed line marked with the asterisk in formula (a), (b), (c), (d), (e), (f), (g), (h), (i) and (j) indicates attachment to a π-electron-pair-donating heteroaromatic N of axitinib. In certain embodiments, the unmarked dashed line in formula (a), (b), (c), (d), (e), (f), (g), (h), (i) and j) indicates attachment to a hydrogel, in particular to a PEG-based hydrogel.

In certain embodiments, Z is a polymeric moiety.

In certain embodiments, Z is a C₈₋₂₄ alkyl.

In certain embodiments, Z is water-soluble.

In certain embodiments, Z is a water-soluble polymeric moiety.

If Z is a water-soluble polymeric moiety, such polymeric moiety has a molecular weight ranging from and including 1 kDa to 1000 kDa. In certain embodiments, Z has a molecular weight ranging from and including 5 kDa to 1000 kDa. In certain embodiments, Z has a molecular weight ranging from and including 5 kDa to 500 kDa. In certain embodiments, Z has a molecular weight ranging from and including 10 kDa to 250 kDa. In certain embodiments, Z has a molecular weight ranging from and including 10 kDa to 150 kDa. In certain embodiments, Z has a molecular weight ranging from and including 12 kDa to 100 kDa. In certain embodiments, Z has a molecular weight ranging from and including 15 kDa to 80 kDa. In certain embodiments, Z has a molecular weight ranging from and including 10 kDa to 80 kDa.

In certain embodiments, Z has a molecular weight of about 80 kDa. In certain embodiments, Z has a molecular weight of about 70 kDa. In certain embodiments, Z has a molecular weight of about 60 kDa. In certain embodiments, Z has a molecular weight of about 50 kDa. In certain embodiments, Z has a molecular weight of about 40 kDa. In certain embodiments, Z has a molecular weight of about 30 kDa. In certain embodiments, Z has a molecular weight of about 20 kDa. In certain embodiments, Z has a molecular weight of about 10 kDa. In certain embodiments, Z has a molecular weight of about 5 kDa.

In certain embodiments, Z is a water-soluble polymeric moiety comprising a polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans and copolymers thereof.

In certain embodiments, Z is a water-soluble polymeric moiety comprising a protein, such as a protein selected from the group consisting of carboxyl-terminal peptide of the chorionic gonadotropin as described in US 2012/0035101 A1 which are herewith incorporated by reference; albumin; XTEN sequences as described in WO 2011123813 A2 which are herewith incorporated by reference; proline/alanine random coil sequences as described in WO 2011/144756 A1 which are herewith incorporated by reference; proline/alanine/serine random coil sequences as described in WO 2008/155134 A1 and WO 2013/024049 A1 which are herewith incorporated by reference; and Fc-fusion proteins.

In certain embodiments, Z is a polysarcosine. In certain embodiments, Z comprises poly(N-methylglycine). In certain embodiments, Z comprises a random coil protein moiety.

In certain embodiments, such random coil protein moiety comprises at least 25 amino acid residues and at most 2000 amino acids. In certain embodiments, such random coil protein moiety comprises at least 30 amino acid residues and at most 1500 amino acid residues. In certain embodiments, such random coil protein moiety comprises at least 50 amino acid residues and at most 500 amino acid residues.

In certain embodiments, Z comprises a random coil protein moiety of which at least 80%, in certain embodiments at least 85%, in certain embodiments at least 90%, in certain embodiments at least 95%, in certain embodiments at least 98% and in certain embodiments at least 99% of the total number of amino acids forming said random coil protein moiety are selected from alanine and proline. In certain embodiments, at least 10%, but less than 75%, in certain embodiments less than 65% of the total number of amino acid residues of such random coil protein moiety are proline residues. In certain embodiments, such random coil protein moiety is as described in WO 2011/144756 A1, which is hereby incorporated by reference in its entirety. In certain embodiments, Z comprises at least one moiety selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:51 and SEQ ID NO:61 as disclosed in WO2011/144756 which are hereby incorporated by reference. A moiety comprising such random coil protein comprising alanine and proline will be referred to as “PA” or “PA moiety”. Accordingly, in certain embodiments, Z comprises a PA moiety.

In certain embodiments, Z comprises a random coil protein moiety of which at least 80%, in certain embodiments at least 85%, in certain embodiments at least 90%, in certain embodiments at least 95%, in certain embodiments at least 98% and in certain embodiments at least 99% of the total number of amino acids forming said random coil protein moiety are selected from alanine, serine and proline. In certain embodiments, at least 4%, but less than 40% of the total number of amino acid residues of such random coil protein moiety are proline residues. In certain embodiments, such random coil protein moiety is as described in WO 2008/155134 A1, which is hereby incorporated by reference in its entirety. In certain embodiments, Z comprises at least one moiety selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:40, SEQ ID NO:42, SEQ ID NO:44, SEQ ID NO:46, SEQ ID NO:50, SEQ ID NO:52, SEQ ID NO:54 and SEQ ID NO:56 as disclosed in WO 2008/155134 A1, which are hereby incorporated by reference. A moiety comprising such random coil protein moiety comprising alanine, serine and proline will be referred to as “PAS” or “PAS moiety”. Accordingly, in certain embodiments, Z comprises a PAS moiety.

In certain embodiments, Z comprises a random coil protein moiety of which at least 80%, in certain embodiments at least 85%, in certain embodiments at least 90%, in certain embodiments at least 95%, in certain embodiments at least 98% and in certain embodiments 99% of the total number of amino acids forming said random coil protein moiety are selected from alanine, glycine, serine, threonine, glutamate and proline. In certain embodiments, such random coil protein moiety is as described in WO 2010/091122 A1 which is hereby incorporated by reference. In certain embodiments, Z comprises at least one moiety selected from the group consisting of SEQ ID NO:182, SEQ ID NO:183, SEQ ID NO:184; SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:195, SEQ ID NO: 196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:759, SEQ ID NO:760, SEQ ID NO:761, SEQ ID NO:762, SEQ ID NO:763, SEQ ID NO:764, SEQ ID NO:765, SEQ ID NO:766, SEQ ID NO:767, SEQ ID NO:768, SEQ ID NO:769, SEQ ID NO:770, SEQ ID NO:771, SEQ ID NO:772, SEQ ID NO:773, SEQ ID NO:774, SEQ ID NO:775, SEQ ID NO:776, SEQ ID NO:777, SEQ ID NO:778, SEQ ID NO:779, SEQ ID NO:1715, SEQ ID NO:1716, SEQ ID NO:1718, SEQ ID NO:1719, SEQ ID NO:1720, SEQ ID NO:1721 and SEQ ID NO:1722 as disclosed in WO2010/091122A1, which are hereby incorporated by reference. A moiety comprising such random coil protein moiety comprising alanine, glycine, serine, threonine, glutamate and proline will be referred to as “XTEN” or “XTEN moiety” in line with its designation in WO 2010/091122 A1. Accordingly, in certain embodiments, Z comprises an XTEN moiety.

In certain embodiments, Z is a hyaluronic acid-based polymer.

In certain embodiments, Z is a polymeric moiety as disclosed in WO 2013/024047 A1 which is herewith incorporated by reference. In certain embodiments, Z is a polymeric moiety as disclosed in WO 2013/024048 A1 which is herewith incorporated by reference.

In certain embodiments, Z is a PEG-based polymer, such as linear, branched or multi-arm PEG-based polymer.

In certain embodiments, Z is a linear PEG-based polymer.

In certain embodiments, Z is a branched C₈₋₂₄ alkyl having one, two, three, four, five or six branching points. In certain embodiments, Z is a branched C₈₋₂₄ alkyl having one, two or three branching points. In certain embodiments, Z is a branched C₈₋₂₄ alkyl having one branching point. In certain embodiments, Z is a branched C₈₋₂₄ alkyl having two branching points. In certain embodiments, Z is a branched C₈₋₂₄ alkyl having three branching points.

In certain embodiments, Z is a branched polymer. In certain embodiments, Z is a branched polymer having one, two, three, four, five or six branching points. In certain embodiments, Z is a branched polymer having one, two or three branching points. In certain embodiments, Z is a branched polymer having one branching point. In certain embodiments, Z is a branched polymer having two branching points. In certain embodiments, Z is a branched polymer having three branching points.

In certain embodiments, a branching point is selected from the group consisting of —N<, —CH< and >C<.

In certain embodiments, such branched moiety Z is PEG-based.

In certain embodiments, Z is a multi-arm PEG-based polymer.

In certain embodiments, Z is a multi-arm PEG-based polymer having at least 2 PEG-based arms, such as 2, 3, 4, 5, 6, 7, or 8 PEG-based arms.

In certain embodiments, Z is a branched PEG-based polymer comprising at least 10% PEG, has one branching point and two PEG-based polymer arms and has a molecular weight of about 40 kDa. Accordingly, each of the two PEG-based polymer arms has a molecular weight of about 20 kDa. In certain embodiments, the branching point is —CH<.

In certain embodiments, Z is a branched PEG-based polymer comprising at least 10% PEG, has three branching points and four PEG-based polymer arms and has a molecular weight of about 40 kDa. Accordingly, each of the four PEG-based polymer arms has a molecular weight of about 10 kDa. In certain embodiments, each of the three branching points is —CH<.

In certain embodiments, Z is water-insoluble.

In certain embodiments, Z is a water-insoluble polymeric moiety.

In certain embodiments, Z is a water-insoluble polymeric moiety comprising a polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans and copolymers thereof.

In certain embodiments, Z is a hydrogel.

In certain embodiments, Z is a PEG-based or hyaluronic acid-based hydrogel. In certain embodiments, Z is a PEG-based hydrogel. In certain embodiments, Z is a hyaluronic acid-based hydrogel.

In certain embodiments, Z is a hydrogel as described in WO 2006/003014 A2, WO 2011/012715 A1 or WO 2014/056926 A1, which are herewith incorporated by reference in their entirety.

In certain embodiments, Z is a polymer network formed through the physical aggregation of polymer chains, which physical aggregation is preferably caused by hydrogen bonds, crystallization, helix formation or complexation. In certain embodiments, such polymer network is a thermogelling polymer.

In certain embodiments, Z comprises a moiety selected from the group consisting of

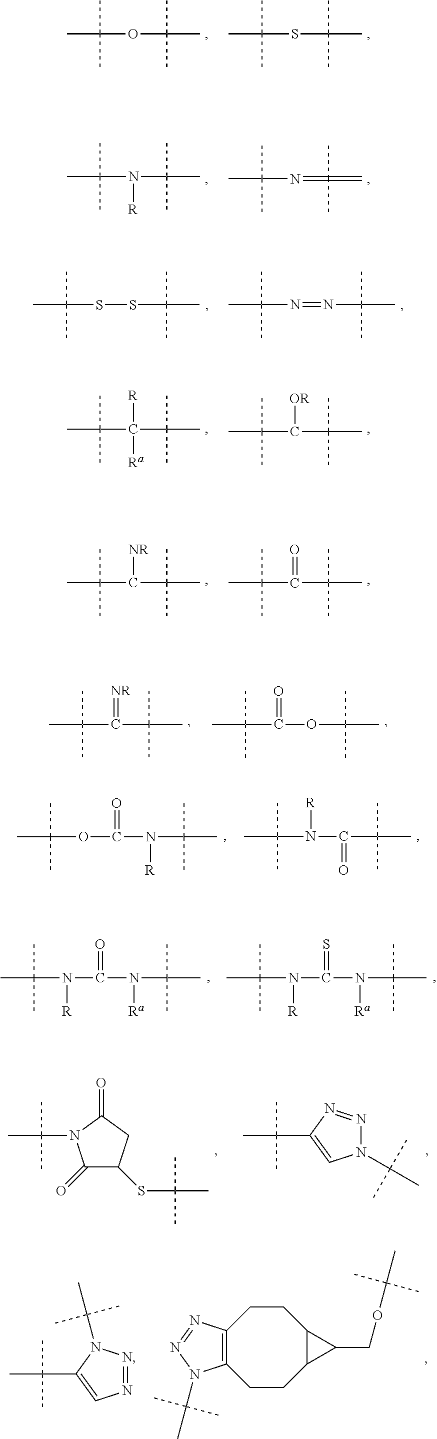

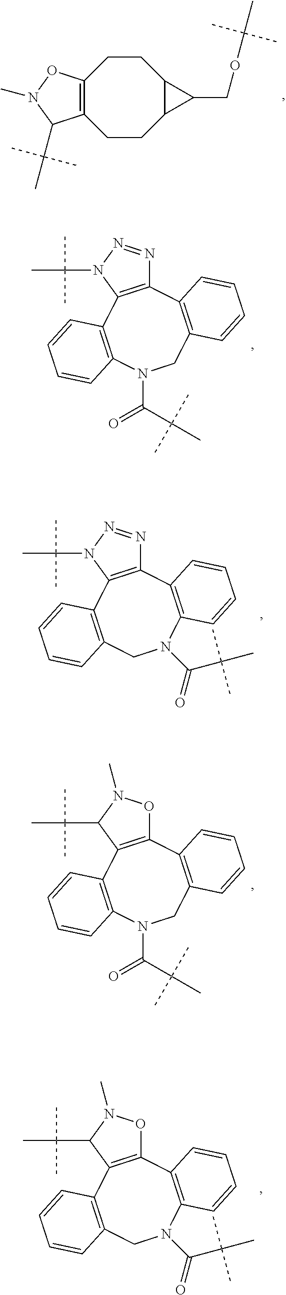

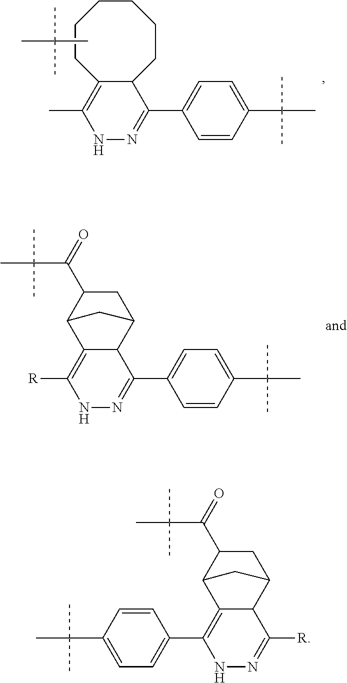

In certain embodiments, the conjugate of the present invention or the pharmaceutically acceptable salt thereof is of formula (Ia), (Ib), (Ic) or (Id):

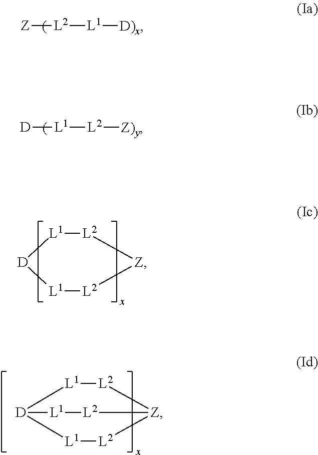

wherein
each -D, -L²- and Z are defined as above and each -L¹- is independently of formula (I);
x is an integer of at least 1; and
y is an integer selected from the group consisting of 2, 3, 4 and 5.

It is understood that even though one -D can be conjugated to multiple -L¹- moieties, the drug moiety is represented by “-D” and the drug by “D-H”.

In certain embodiments, the conjugate is of formula (Ia), (Ic) or (Id) and Z is a hydrogel. In such cases, a plurality of moieties -L²-L¹-D are conjugated to Z and it is understood that no upper limit for x can be provided.

In certain embodiments, the conjugate is of formula (Ia). In certain embodiments, the conjugate is of formula (Ib). In certain embodiments, the conjugate is of formula (Ic). In certain embodiments, the conjugate is of formula (Id). In certain embodiments, the conjugate is of formula (Ia) and Z is a hydrogel.

In certain embodiments, the conjugate is of formula (Ia), (Ic) or (Id), Z is a water-soluble polymeric moiety and x ranges from 2 to 1000, such as from 2 to 1500, such as from 2 to 1000, such as from 2 to 500, such as from 2 to 250 or such as from 2 to 100. In certain embodiments, the conjugate is of formula (Ia), (Ic) or (Id), Z is a water-soluble polymeric moiety and x is 20.

In certain embodiments, the conjugate is of formula (Ia), (Ic) or (Id), Z is a water-soluble polymeric moiety and x is 19. In certain embodiments, the conjugate is of formula (Ia), (Ic) or (Id), Z is a water-soluble polymeric moiety and x is 18. In certain embodiments, the conjugate is of formula (Ia), (Ic) or (Id), Z is a water-soluble polymeric moiety and x is 17. In certain embodiments, the conjugate is of formula (Ia), (Ic) or (Id), Z is a water-soluble polymeric moiety and x is 16. In certain embodiments, the conjugate is of formula (Ia), (Ic) or (Id), Z is a water-soluble polymeric moiety and x is 15. In certain embodiments, the conjugate is of formula (Ia), (Ic) or (Id), Z is a water-soluble polymeric moiety and x is 14. In certain embodiments, the conjugate is of formula (Ia), (Ic) or (Id), Z is a water-soluble polymeric moiety and x is 13. In certain embodiments, the conjugate is of formula (Ia), (Ic) or (Id), Z is a water-soluble polymeric moiety and x is 12. In certain embodiments, the conjugate is of formula (Ia), (Ic) or (Id), Z is a water-soluble polymeric moiety and x is 11. In certain embodiments, the conjugate is of formula (Ia), (Ic) or (Id), Z is a water-soluble polymeric moiety and x is 10. In certain embodiments, the conjugate is of formula (Ia), (Ic) or (Id), Z is a water-soluble polymeric moiety and x is 9. In certain embodiments, the conjugate is of formula (Ia), (Ic) or (Id), Z is a water-soluble polymeric moiety and x is 8. In certain embodiments, the conjugate is of formula (Ia), (Ic) or (Id), Z is a water-soluble polymeric moiety and x is 7. In certain embodiments, the conjugate is of formula (Ia), (Ic) or (Id), Z is a water-soluble polymeric moiety and x is 6. In certain embodiments, the conjugate is of formula (Ia), (Ic) or (Id), Z is a water-soluble polymeric moiety and x is 5. In certain embodiments, the conjugate is of formula (Ia), (Ic) or (Id), Z is a water-soluble polymeric moiety and x is 4. In certain embodiments, the conjugate is of formula (Ia), (Ic) or (Id), Z is a water-soluble polymeric moiety and x is 3. In certain embodiments, the conjugate is of formula (Ia), (Ic) or (Id), Z is a water-soluble polymeric moiety and x is 2.

In certain embodiments, the conjugate is of formula (Ib), Z is a water-soluble polymeric moiety and y is 1. In certain embodiments, the conjugate is of formula (Ib), Z is a water-soluble polymeric moiety and y is 2. In certain embodiments, the conjugate is of formula (Ib), Z is a water-soluble polymeric moiety and y is 3. In certain embodiments, the conjugate is of formula (Ib), Z is a water-soluble polymeric moiety and y is 4. In certain embodiments, the conjugate is of formula (Ib), Z is a water-soluble polymeric moiety and y is 5.

In certain embodiments, -L¹- of formula (I) is of formula (Ix):

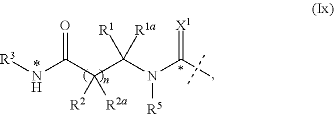

wherein the dashed line indicates the attachment to the π-electron-pair-donating heteroaromatic N of -D;
═X¹, —R, —R^{1a}, —R², —R^{2a}, —R³, —R⁵ and n are used as defined in formula (I);
optionally, one or more of the pairs —R¹/—R^{1a}, —R²/—R^{2a}, two adjacent —R² are joined together with the atom to which they are attached to form a C₃₋₁₀ cycloalkyl, 3- to 10-membered heterocyclyl or an 8- to 11-membered heterobicyclyl;
optionally, one or more of the pairs —R¹/—R², —R¹/—R⁵, —R²/—R⁵ and —R⁴/—R⁵ are joined together with the atoms to which they are attached to form a ring -A-;
   wherein -A- is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl;
optionally, —R¹ and an adjacent —R² form a carbon-carbon double bond provided that n is selected from the group consisting of 1, 2, 3 and 4;
optionally, two adjacent —R² form a carbon-carbon double bond provided that n is selected from the group consisting of 2, 3 and 4;
and wherein the distance between the nitrogen atom marked with an asterisk and the carbon atom marked with an asterisk in formula (Ix) is 5, 6 or 7 atoms and if present the carbon-carbon double bond formed between —R¹ and —R² or two adjacent —R² is in a cis configuration.

In certain embodiments, n of formula (Ix) is 0. In certain embodiments, n of formula (Ix) is 1. In certain embodiments, n of formula (Ix) is 2.

In certain embodiments, —R¹ and —R^{1a} of formula (Ix) are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. In this case it is understood that —R¹/—R^{1a} may optionally be joined together with the atom to which they are attached to form a C₃₋₁₀ cycloalkyl and that one or more of the pairs —R¹/—R² and —R¹/—R⁵ may optionally be joined together with the atoms to which they are attached to form a ring -A-, wherein -A- is used as defined for formula (I).

In certain embodiments, —R² and —R^{2a} of formula (Ix) are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. In this case it is understood that one or more of the pairs —R²/—R^{2a} and two adjacent —R² may optionally be joined with the atom to which they are attached to form a C₃₋₁₀ cycloalkyl and that the pair —R²/—R⁵ may optionally be joined together with the atoms to which they are attached to form a ring -A-, wherein -A- is used as defined in formula (I).

In certain embodiments, ═X¹ of formula (Ix) is ═O.

In certain embodiments, —R¹ and —R^{1a} of formula (Ix) are both —H.

In certain embodiments, —R¹ of formula (Ix) is —H and —R^{1a} of formula (Ix) is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

In certain embodiments, —R³ of formula (Ix) is C₁₋₆ alkyl.

In certain embodiments, —R⁵ of formula (Ix) is —H. In certain embodiments, —R⁵ of formula (Ix) is methyl. In certain embodiments, —R⁵ of formula (Ix) is ethyl.

In certain embodiments, —R⁷ of formula (Ix) is hydrogen. In certain embodiments, —R⁷ of formula (Ix) is methyl. In certain embodiments, —R⁷ of formula (Ix) is ethyl.

In certain embodiments, -L¹- of formula (I) is of formula (I′):

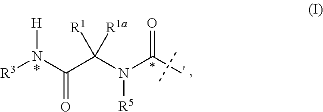

wherein the dashed line indicates the attachment to the π-electron-pair-donating heteroaromatic N of -D;
—R¹, —R^{1a}, —R³ and —R⁵ are used as defined in formula (I);
optionally, the pair —R¹/—R^{1a} is joined together with the atom to which they are attached to form a C₃₋₁₀ cycloalkyl, 3- to 10-membered heterocyclyl or an 8- to 11-membered heterobicyclyl; and
optionally, the pair —R¹/—R⁵ is joined together with the atoms to which they are attached to form a 3- to 10-membered heterocyclyl or 8- to 11-membered heterobicyclyl.

In certain embodiments, —R¹ and —R^{1a} of formula (I′) are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. In this case it is understood that —R¹/—R^{1a} may optionally be joined together with the atom to which they are attached to form a C₃₋₁₀ cycloalkyl and that the pairs —R¹/—R⁵ may optionally be joined together with the atoms to which they are attached to form a 3- to 10-membered heterocyclyl or 8- to 11-membered heterobicyclyl.

In certain embodiments, —R¹ and —R^{1a} of formula (I′) are both —H.

In certain embodiments, —R¹ of formula (I′) is —H and —R^{1a} of formula (I′) is C₁₋₆ alkyl. In certain embodiments, —R¹ of formula (I′) is —H and —R^{1a} of formula (I′) is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

In certain embodiments, —R³ of formula (I′) is C₁₋₆ alkyl.

In certain embodiments, —R⁵ of formula (I′) is methyl. In certain embodiments, —R⁵ of formula (I′) is ethyl.

In certain embodiments, —R⁵ of formula (I′) is —CH₃, —R¹ and —R^{1a} of formula (I′) are —H and —R³ of formula (I′) is —H which is replaced by one -L²-Z moiety.

In certain embodiments, —R⁵ of formula (I′) is —CH₃, —R¹ of formula (I′) is —H and —R^{1a} of formula (I′) is —CH₃ and —R³ of formula (I′) is —H which is replaced by one -L²-Z moiety.

In certain embodiments, —R⁵ of formula (I′) is ethyl, —R¹ and —R^{1a} of formula (I′) are —H and —R³ of formula (I′) is —H which is replaced by one -L²-Z moiety.

In certain embodiments, -L¹- of formula (I) is of formula (Iy):

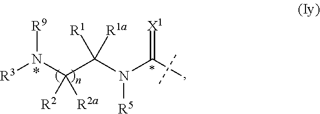

wherein the dashed line indicates the attachment to the π-electron-pair-donating heteroaromatic N of -D;
═X¹, —R¹, —R^{1a}, —R², —R^{2a}, —R³, —R⁵, —R⁹ and n are used as defined in formula (I);
optionally, one or more of the pairs —R¹/—R^{1a}, —R²/—R^{2a}, two adjacent —R² and —R³/—R⁹ are joined together with the atom to which they are attached to form a C₃₋₁₀ cycloalkyl, 3- to 10-membered heterocyclyl or an 8- to 11-membered heterobicyclyl;
optionally, one or more of the pairs —R¹/—R², —R¹/—R⁵, —R²/—R⁵ and —R⁴/—R⁵ are joined together with the atoms to which they are attached to form a ring -A-;
   wherein -A- is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl;
optionally, —R¹ and an adjacent —R² form a carbon-carbon double bond provided that n is selected from the group consisting of 1, 2, and 3;
optionally, two adjacent —R² form a carbon-carbon double bond provided that n is selected from the group consisting of 2, and 3;
and wherein the distance between the nitrogen atom marked with an asterisk and the carbon atom marked with an asterisk in formula (Iy) is 5, 6 or 7 atoms and if present the carbon-carbon double bond formed between —R¹ and —R² or two adjacent —R² is in a cis configuration.

In certain embodiments, n of formula (Iy) is 1. In certain embodiments, n of formula (Iy) is 2.

In certain embodiments, n of formula (Iy) is 3.

In certain embodiments, —R¹ and —R^{1a} of formula (Iy) are independently selected from the group consisting of —H and C₁₋₆ alkyl. In certain embodiments, —R¹ and —R^{1a} of formula (Iy) are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. In this case it is understood that —R¹/—R^{1a} may optionally be joined together with the atom to which they are attached to form a C₃₋₁₀ cycloalkyl and that one or more of the pairs —R¹/—R⁵, —R¹/—R⁹ and —R¹/—R¹⁰ may optionally be joined together with the atoms to which they are attached to form a ring -A-, wherein -A- is used as defined for formula (I).

In certain embodiments, —R¹ and —R^{1a} of formula (Iy) are both —H.

In certain embodiments, —R² and —R^{2a} of formula (Iy) are independently selected from the group consisting of —H and C₁₋₆ alkyl. In certain embodiments, —R² and —R^{2a} of formula (Iy) are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. In this case it is understood that one or more of the pairs —R²/—R^{2a} and two adjacent —R² may optionally be joined with the atom to which they are attached to form a C₃₋₁₀ cycloalkyl and that the pair —R²/—R⁵ may optionally be joined together with the atoms to which they are attached to form a 3- to 10-membered heterocyclyl or 8- to 11-membered heterobicyclyl.

In certain embodiments, —R² and —R^{2a} of formula (Iy) are both —H.

In certain embodiments, —R³ of formula (Iy) is H. In certain embodiments, —R³ of formula (Iy) is methyl.

In certain embodiments, —R⁵ of formula (Iy) is H. In certain embodiments, —R⁵ of formula (Iy) is methyl.

The conjugates of the present invention release one or more types of drug over an extended period of time, i.e. they are sustained-release conjugates. In certain embodiments, the release occurs with a release half-life ranging between 1 day and 1 month. In certain embodiments, the release occurs with a release half-life ranging between 1 day and 20 days. In certain embodiments, the release occurs with a release half-life between 1 day and 15 days. In certain embodiments the release half-life may also range from 2 to 20 days, 4 to 15 days or 3 to 6 days.

Another aspect of the present invention is a pharmaceutical composition comprising at least one conjugate of the present invention or a pharmaceutical salt thereof.

In certain embodiments, the pharmaceutical composition comprises one conjugate of the present invention or a pharmaceutical salt thereof. In certain embodiments, the pharmaceutical composition comprises two conjugates of the present invention. In certain embodiments, the pharmaceutical composition comprises three conjugates of the present invention.

Such pharmaceutical composition may have a pH ranging from pH 3 to pH 8, such as ranging from pH 4 to pH 6 or ranging from pH 4 to pH 5. In certain embodiments, the pH of the pharmaceutical composition is about 4. In certain embodiments, the pH of the pharmaceutical composition is about 4.5. In certain embodiments, the pH of the pharmaceutical composition is about 5. In certain embodiments, the pH of the pharmaceutical composition is about 5.5.

In certain embodiments, the pH of the pharmaceutical composition is 4. In certain embodiments, the pH of the pharmaceutical composition is 4.5. In certain embodiments, the pH of the pharmaceutical composition is 5. In certain embodiments, the pH of the pharmaceutical composition is 5.5.

In certain embodiments, such pharmaceutical composition is a suspension formulation.

In certain embodiments such pharmaceutical is a dry composition. It is understood that such dry composition may be obtained by drying, such as lyophilizing, a suspension composition.

If the pharmaceutical composition is a parenteral composition, suitable excipients may be categorized as, for example, buffering agents, isotonicity modifiers, preservatives, stabilizers, anti-adsorption agents, oxidation protection agents, viscosifiers/viscosity enhancing agents, anti-agglomeration agents or other auxiliary agents. However, in some cases, one excipient may have dual or triple functions. Excipient may be selected from the group consisting of

(i) Buffering agents: physiologically tolerated buffers to maintain pH in a desired range, such as sodium phosphate, bicarbonate, succinate, histidine, citrate, acetate, sulphate, nitrate, chloride, or pyruvate; antacids such as Mg(OH)₂ or ZnCO₃ may be also used;
(ii) Isotonicity modifiers: to minimize pain that can result from cell damage due to osmotic pressure differences at the injection depot; glycerin and sodium chloride are examples; effective concentrations can be determined by osmometry using an assumed osmolality of 285-315 mOsmol/kg for serum;
(iii) Preservatives and/or antimicrobials: multidose parenteral formulations require the addition of preservatives at a sufficient concentration to minimize risk of patients becoming infected upon injection and corresponding regulatory requirements have been established; typical preservatives include m-cresol, phenol, methylparaben, ethylparaben, propylparaben, butylparaben, chlorobutanol, benzyl alcohol, phenylmercuric nitrate, thimerosol, sorbic acid, potassium sorbate, benzoic acid, chlorocresol and benzalkonium chloride;
(iv) Stabilizers: Stabilisation is achieved by strengthening of the protein-stabilising forces, by destabilisation of the denatured state, or by direct binding of excipients to the protein; stabilizers may be amino acids such as alanine, arginine, aspartic acid, glycine, histidine, lysine, proline, sugars such as glucose, sucrose, trehalose, polyols such as glycerol, mannitol, sorbitol, salts such as potassium phosphate, sodium sulphate, chelating agents such as EDTA, hexaphosphate, ligands such as divalent metal ions (zinc, calcium, etc.), other salts or organic molecules such as phenolic derivatives; in addition, oligomers or polymers such as cyclodextrins, dextran, dendrimers, PEG or PVP or protamine or HSA may be used;
(v) Anti-adsorption agents: Mainly ionic or non-ionic surfactants or other proteins or soluble polymers are used to coat or adsorb competitively to the inner surface of the formulation's container; e.g., poloxamer (Pluronic F-68), PEG dodecyl ether (Brij 35), polysorbate 20 and 80, dextran, polyethylene glycol, PEG-polyhistidine, BSA and HSA and gelatins; chosen concentration and type of excipient depends on the effect to be avoided but typically a monolayer of surfactant is formed at the interface just above the CMC value;
(vi) Oxidation protection agents: antioxidants such as ascorbic acid, ectoine, methionine, glutathione, monothioglycerol, morin, polyethylenimine (PEI), propyl gallate, and vitamin E; chelating agents such as citric acid, EDTA, hexaphosphate, and thioglycolic acid may also be used;
(vii) Viscosifiers or viscosity enhancers: retard settling of the particles in the vial and syringe and are used in order to facilitate mixing and resuspension of the particles and to make the suspension easier to inject (i.e., low force on the syringe plunger); suitable viscosifiers or viscosity enhancers are, for example, carbomer viscosifiers like Carbopol 940, Carbopol Ultrez 10, cellulose derivatives like hydroxypropylmethylcellulose (hypromellose, HPMC) or diethylaminoethyl cellulose (DEAE or DEAE-C), colloidal magnesium silicate (Veegum) or sodium silicate, hydroxyapatite gel, tricalcium phosphate gel, xanthans, carrageenans like Satia gum UTC 30, aliphatic poly(hydroxy acids), such as poly(D,L- or L-lactic acid) (PLA) and poly(glycolic acid) (PGA) and their copolymers (PLGA), terpolymers of D,L-lactide, glycolide and caprolactone, poloxamers, hydrophilic poly(oxyethylene) blocks and hydrophobic poly(oxypropylene) blocks to make up a triblock of poly(oxyethylene)-poly(oxypropylene)-poly(oxyethylene) (e.g. Pluronic®), polyetherester copolymer, such as a polyethylene glycol terephthalate/polybutylene terephthalate copolymer, sucrose acetate isobutyrate (SAIB), dextran or derivatives thereof, combinations of dextrans and PEG, polydimethylsiloxane, collagen, chitosan, polyvinyl alcohol (PVA) and derivatives, polyalkylimides, poly (acrylamide-co-diallyldimethyl ammonium (DADMA)), polyvinylpyrrolidone (PVP), glycosaminoglycans (GAGs) such as dermatan sulfate, chondroitin sulfate, keratan sulfate, heparin, heparan sulfate, hyaluronan, ABA triblock or AB block copolymers composed of hydrophobic A-blocks, such as polylactide (PLA) or poly(lactide-co-glycolide) (PLGA), and hydrophilic B-blocks, such as polyethylene glycol (PEG) or polyvinyl pyrrolidone; such block copolymers as well as the abovementioned poloxamers may exhibit reverse thermal gelation behavior (fluid state at room temperature to facilitate administration and gel state above sol-gel transition temperature at body temperature after injection);
(viii) Spreading or diffusing agent: modifies the permeability of connective tissue through the hydrolysis of components of the extracellular matrix in the intrastitial space such as but not limited to hyaluronic acid, a polysaccharide found in the intercellular space of connective tissue; a spreading agent such as but not limited to hyaluronidase temporarily decreases the viscosity of the extracellular matrix and promotes diffusion of injected drugs;
(ix) Anti-agglomeration agents, such as propylene glycol; and
(x) Other auxiliary agents: such as wetting agents, viscosity modifiers, antibiotics, hyaluronidase; acids and bases such as hydrochloric acid and sodium hydroxide are auxiliary agents necessary for pH adjustment during manufacture.

In another aspect, the present invention relates to a conjugate of the present invention or a pharmaceutical composition comprising a conjugate of the present invention for use as a medicament.

In another aspect, the present invention relates to a conjugate or a pharmaceutically acceptable salt thereof of the present invention or a pharmaceutical composition comprising a conjugate of the present invention for use in a method of treating a disease that can be treated with D-H or its pharmaceutically acceptable salt thereof.

In a further aspect, the present invention relates to a method of preventing a disease or treating a patient suffering from a disease that can be prevented or treated with D-H comprising administering an effective amount of the conjugate or its pharmaceutically acceptable salt thereof of the present invention or the pharmaceutical compositions comprising said conjugates to the patient.

As the present invention is applicable to all drug molecules comprising a π-electron-pair-donating heteroaromatic N, it is impossible to further specify the disease that can be treated. However, it is evident to the person skilled in the art which disease can be treated with a particular conjugate.

### EXAMPLES

### Materials and Methods

All materials were commercially available except where stated otherwise.

### Amino Hydrogels

PEG based amino hydrogels were synthesized as described in example 3 of WO2011/012715A1 using different crosslinkers and crosslinking degrees to give different levels of amine content. All crosslinkers were based on 2 kDa PEG and were synthesized as described in example 2 of WO2011/012715A1 using adipic acid (C6), suberic acid (C8), or azelaic acid (C9). The choice of crosslinker is in brackets and the hydrogels were characterized by their free amine content: HG-1: 0.309 mmol/g (C6), HG-2: 0.300 mmol/g (C6), HG-3: 0.134 mmol/g (C6); HG-4: 0.668 mmol/g (C9); HG-5: 0.303 mmol/g (C6); HG-6: 0.668 mmol/g (C9); HG-7: 0.331 mmol/g (C6); HG-8: 0.686 mmol/g (C9); HG-9: 0.393 mmol/g; (C9): HG-10: 0.474 mmol/g (C8); HG-16: 0.483 mmol/g (C9)

The following hydrogels were prepared by modification of amine hydrogels with lysine as described in example 5 of WO2011/042450A1, and were characterised by their free amine content:

HG-11: 0564 mmol/g (from HG-5); HG-12: 0.614 mmol/g (from HG-7), HG-13: 0.691 mmol/g (from HG-9), HG-14: 0.934 mmol/g (from HG-10). HG-15: 0.621 mmol/g (from HG-7); HG-17: 0.864 mmol/g (from HG-16)

### Reactions

Reactions were performed with dry solvents (CH₂Cl₂, DMF, THF) stored over molecular sieves purchased from Sigma-Aldrich Chemie GmbH, Munich, Germany. Generally, reactions were stirred at room temperature and monitored by LCMS.

### Solid Phase Synthesis

Solid phase synthesis was performed in syringe reactors with frit. A standard Fmoc protocol was used. 2-Chlorotrityl chloride resin (100-200 mesh), 1% DVB (Merck, Darmstadt, Germany) was loaded with the first amino acid using DIPEA in DCM. Fmoc deprotection was performed using 2:2:96 piperidine/DBU/DMF. Coupling of the next amino acid was performed using PyBOP/DIPEA or HATU/DIPEA in DMF. Cleavage from the resin was performed using HFIP or TFA/TES/water/DCM 48:2:2:48. Products were concentrated in vacuo.

### RP-HPLC Purification

Preparative RP-HPLC purifications were performed with a Waters 600 controller with a 2487 Dual Absorbance Detector or an Agilent Infinity 1260 preparative system using a Waters XBridge BEH300 Prep C18 10 μm, 150×30 mm column as stationary phase. Products were detected at 215 nm, 320 nm or 360 nm. Linear gradients of solvent system A (water containing 0.1% TFA v/v) and solvent system B (acetonitrile containing 0.1% TFA v/v) were used. HPLC fractions containing product were pooled and lyophilized if not stated otherwise.

### Flash Chromatography

Flash chromatography purifications were performed on an Isolera One system or an Isolera Four system from Biotage AB, Sweden, using Biotage KP-Sil silica cartridges. Products were detected at 254 nm, 280 nm, or 360 nm.

### RP-LPLC Purification

Low pressure RP chromatography purifications were performed on an Isolera One system or an Isolera Four system from Biotage AB, Sweden, using Biotage SNAP C18 cartridges. Products were detected at 215 nm and 360 nm. Linear gradients of solvent system A (water containing 0.1% TFA v/v) and solvent system B (acetonitrile containing 0.1% TFA v/v). LPLC fractions containing product were pooled and lyophilized if not stated otherwise.

UPLC-MS Analysis

Analytical ultra-performance LC (UPLC)-MS was performed on a Waters Acquity system or an Agilent 1290 Infinity II equipped with a Waters BEH300 C18 column (2.1×50 mm, 1.7 μm particle size or 2.1×100 mm, 1.7 μm particle size); solvent A: water containing 0.04% TFA (v/v), solvent B: acetonitrile containing 0.05% TFA (v/v) coupled to a Waters Micromass ZQ or coupled to an Agilent Single Quad MS system.
Drug Moiety Content Determination from Hydrogels

Drug moiety contents of hydrogels were determined by total release of the drug after basic incubation and LCMS quantification (UV based).

### Example 1: Synthesis of Indazole Conjugate 1e

Linker reagent 1e was synthesized according to the following scheme:

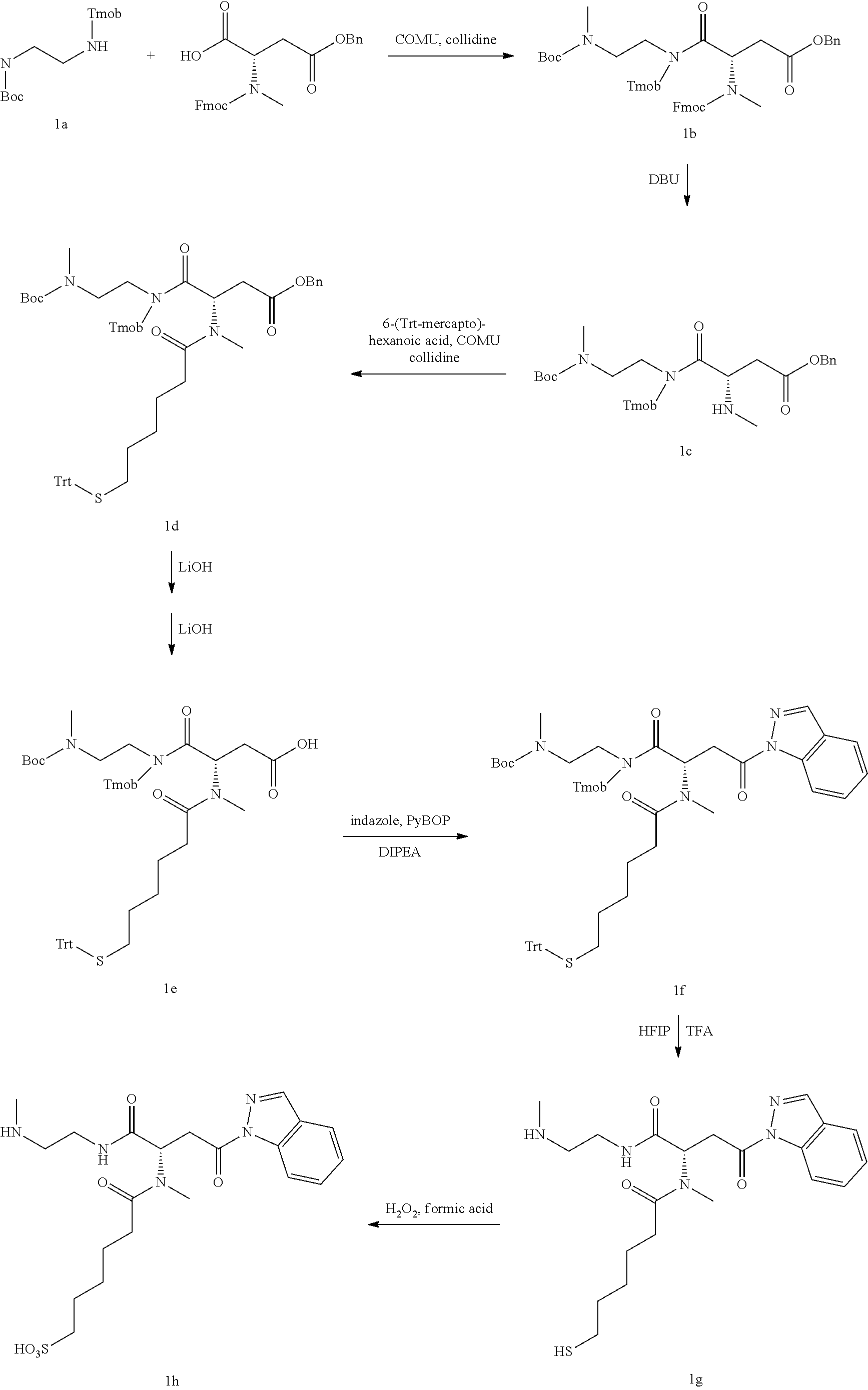

To a solution of N-Methyl-N-boc-ethylenediamine (2 g, 11.48 mmol) and NaCNBH₃ (819 mg, 12.63 mmol) in MeOH (20 mL) was added 2,4,6-trimethoxybenzaldehyde (2.08 mg, 10.61 mmol) portion-wise. The mixture was stirred at RT for 90 min, acidified with 3 M HCl (4 mL) and stirred further 15 min. The reaction mixture was added to saturated NaHCO₃ solution (200 mL) and extracted 5× with CH₂Cl₂. The combined organic phases were dried over Na₂SO₄ and the solvents were evaporated in vacuo. The resulting N-Methyl-N-boc-N′-tmob-ethylenediamine 1a was dried in high vacuum and used in the next reaction step without further purification.

Yield: 3.76 g (11.48 mmol, 89% purity, 1a: double Tmob protected product=8:1)

MS: m/z 355.22=[M+H]⁺, (calculated=354.21).

To a solution of 1a (2 g, 5.65 mmol) in CH₂Cl₂ (24 ml) COMU (4.84 g, 11.3 mmol), N-Fmoc-N-Me-Asp(OBn)-OH (2.08 g, 4.52 mmol) and collidine (2.65 mL, 20.34 mmol) were added. The reaction mixture was stirred for 3 h at RT, diluted with CH₂Cl₂ (250 mL) and washed 3× with 0.1 M H₂SO₄ (100 mL) and 3× with brine (100 mL). The aqueous phases were re-extracted with CH₂Cl₂ (100 mL). The combined organic phases were dried over Na₂SO₄, filtrated and the residue concentrated to a volume of 24 mL. 1b was purified using flash chromatography.

Yield: 5.31 g (148%, 6.66 mmol)

MS: m/z 796.38=[M+H]+, (calculated=795.37).

To a solution of 1b (5.31 g, max. 4.51 mmol ref. to N-Fmoc-N-Me-Asp(OBn)-OH) in THF (60 mL) DBU (1.8 mL, 3% v/v) was added. The solution was stirred for 12 min at RT, diluted with CH₂Cl₂ (400 ml) and washed 3× with 0.1 M H₂SO₄ (150 ml) and 3× with brine (150 ml). The aqueous phases were re-extracted with CH₂Cl₂ (100 ml). The combined organic phases were dried over Na₂SO₄ and filtrated. 1c was isolated upon evaporation of the solvent and used in the next reaction without further purification.

MS: m/z 574.31=[M+H]⁺, (calculated=573.30).

1c (5.31 g, 4.51 mmol, crude) was dissolved in acetonitrile (26 mL) and COMU (3.87 g, 9.04 mmol), 6-tritylmercaptohexanoic acid (2.12 g, 5.42 mmol) and collidine (2.35 mL, 18.08 mmol) were added. The reaction mixture was stirred for 4 h at RT, diluted with CH₂Cl₂ (400 mL) and washed 3× with 0.1 M H₂SO₄ (100 mL) and 3× with brine (100 mL). The aqueous phases were re-extracted with CH₂Cl₂ (100 ml). The combined organic phases were dried over Na₂SO₄, filtrated and 1d was isolated upon evaporation of the solvent. Product 1d was purified using flash chromatography.

Yield: 2.63 g (62%, 94% purity)

MS: m/z 856.41=[M+H]⁺, (calculated=855.41).

To a solution of 1d (2.63 g, 2.78 mmol) in i-PrOH (33 mL) and H₂O (11 mL) was added LiOH (267 mg, 11.12 mmol) and the reaction mixture was stirred for 70 min at RT. The mixture was diluted with CH₂Cl₂ (200 ml) and washed 3× with 0.1 M H₂SO₄ (50 ml) and 3× with brine (50 ml). The aqueous phases were re-extracted with CH₂Cl₂ (100 mL). The combined organic phases were dried over Na₂SO₄, filtrated and 1e was isolated upon evaporation of the solvent. 1e was purified using flash chromatography.

Yield: 2.1 g (88%)

MS: m/z 878.4=[M+Na]⁺, (calculated=878.40).

Indazole (50 mg, 0.42 mmol) and PyBOP (264 mg, 0.51 mmol) were dissolved in DMF (1.5 mL). To the solution 1e (435 mg, 0.51 mmol) and DIPEA (222 μL, 1.27 mmol) were added with stirring. After 18 h the reaction solution was transferred into a separating funnel, diluted with 10 mL ethyl acetate and the organic phase was washed 1× with 10 mL 0.1 N HCl, 1×10 mL water and 1×10 mL brine. The organic phase was dried over Na₂SO₄, filtered and all volatiles were evaporated. 1f was purified by flash chromatography.

Yield: 173 mg (43%)

MS: m/z 956.72=[M+H]⁺, (calculated=956.47).

1f (80 mg, 84 μmol) was dissolved in HFIP/TES/H₂O 39/1/1 (v/v/v) (1 mL). To the solution TFA (200 μL, 2.6 mmol) was added. All volatiles were removed in a stream of argon. Crude 1g was purified by RP-HPLC.

Yield: 9.6 mg (21%) TFA salt

MS: m/z 434.51=[M+H]⁺, (calculated=434.22).

A mixture of 450 μL formic acid and 50 μL hydrogen peroxide was incubated at RT for 1 h and precooled in the fridge. 100 μL of this solution was added to 1g (2.50 mg; 4.6 μmol). After 10 min 100 μL water were added and the product 1h isolated by repetitive lyophilization.

Yield: 2 mg (86%) formic acid salt

MS: m/z 482.46=[M+H]⁺, (calculated=482.21).

### Example 2: Synthesis of 1-((4-nitrophenoxy)carbonyl)-1H-indazole-3-carboxylic acid 2

1H-indazole-3-carboxylic acid (249 mg, 1.54 mmol) was suspended in DCM (5 mL) and a solution of 4-nitrophenyl chloroformate (343 mg; 1.70 mmol) in DCM (5 mL) was added with stirring. A suspension was obtained. TEA (645 μL, 4.63 mmol) was added with stirring. The reaction solution was diluted after 2 h with 150 ml ethyl acetate and the organic phase was washed 3× with 50 mL 0.1 M HCl. The aqueous phase was re-extracted 2× with 50 mL of ethyl acetate. The combined organic phase was dried over Na₂SO₄, filtered and the solvent was evaporated. The product 1-((4-nitrophenoxy)carbonyl)-1H-indazole-3-carboxylic acid 2 was used without further purification.

Yield: 486 mg (97%)

MS: m/z 327.99=[M+H]⁺, (calculated=328.06).

### Example 3: Synthesis of Compound 3

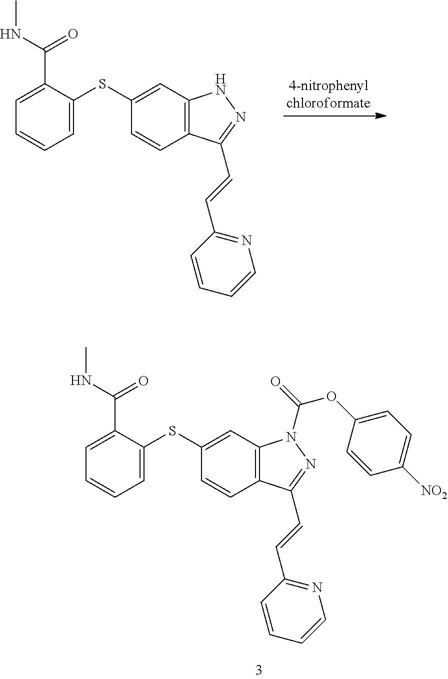 [SMILES: CNC(=O)c1ccccc1Sc1ccc2c(/C=C/c3ccccn3)n[nH]c2c1 ; CNC(=O)c1ccccc1Sc1ccc2c(/C=C/c3ccccn3)nn(C(=O)Oc3ccc([N+](=O)[O-])cc3)c2c1]

4-nitrophenyl chloroformate (188 mg, 0.93 mmol) was dissolved in THF (8 mL). This solution was added to axitinib (100 mg, 0.26 mmol) and the reaction heated at 80° C. for 7 h with stirring (yellow suspension). The reaction suspension was left standing at RT overnight. The suspension was centrifuged, the supernatant removed, and the precipitate washed with ethyl acetate (2 times 6 ml). The precipitate was dried in high vacuum.

Yield: 139 mg (92%, HCl salt)

MS: m/z 552.11=[M+H]⁺, (calculated=552.14).

### Example 4: Synthesis of Compound 4a

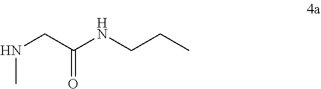

N-benzyloxycarbonyl sarcosine (100 mg, 0.45 mmol) and HOBt (59 mg, 0.44 mmol) were suspended in DCM (1 mL). 1-propylamine (44 μL, 0.54 mmol) was added and a solution was obtained. EDC HCl (91 mg, 0.48 mmol) was added and the reaction stirred. After 4.5 h the reaction was concentrated in vacuo and the crude purified by RP-HPLC. The product was dissolved in THF (1.95 mL) by ultrasonication. To the solution 10% Palladium on activated charcoal (4.2 mg, 0.04 mmol) was added and the reaction was stirred in an atmosphere of hydrogen. After 3 h the reaction was filtered and the filtrate concentrated in vacuo.

Yield: 50 mg (86%)

MS: m/z 130.93=[M+H]⁺, (calculated=131.12).

### Example 5: Synthesis of Compound 4b

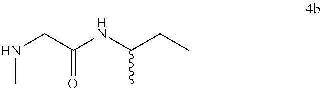

N-benzyloxycarbonyl sarcosine (103 mg, 0.46 mmol) was dissolved in DMF (1 mL) and 2-butylamine (54 μL, 0.53 mmol) and PyBOP (257 mg, 0.49 mmol) were added with stirring. A solution was obtained. DIPEA (156 μL, 0.90 mmol) was added. After 5 h the reaction was quenched with TFA (50 μl) and the product purified by RP-HPLC. The product step was dissolved in THF (2 mL). To the solution 10% Palladium on activated charcoal (4.5 mg, 0.04 mmol) was added and the reaction was stirred in an atmosphere of hydrogen. After 3 h the reaction was filtered, and the filtrate concentrated in vacuo.

Yield: 67 mg (quant)

MS: m/z 144.97=[M+H]⁺, (calculated=145.14).

### Example 6: Synthesis of Compound 4c

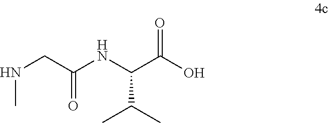

Boc-Sar-OH (99 mg, 0.52 mmol) was dissolved in DCM (1 mL). L-Valine tert-butyl ester hydrochloride (111 mg, 0.53 mmol), EDC HCl (109 mg, 0.57 mmol) and DIPEA (276 μL, 1.59 mmol) were added with stirring. After 3 h the reaction was diluted with 30 mL of DCM and was washed 3 times with 30 mL of 0.1 N HCl, 2 times with sat. NaHCO3 and once with brine. The organic phase was dried over Na₂SO₄, filtered and evaporated. The product was purified by RP-HPLC. The product was dissolved in 0.5 ml of DCM. 0.5 ml of TFA were added with stirring in an open flask. After 5 h the reaction was concentrated in a stream of nitrogen and the product co-evaporated 3 times with DCM.

Yield: 44 mg (28%, TFA salt)

MS: m/z 188.88=[M+H]⁺, (calculated=189.13).

### Example 7: Synthesis of Compound 4d

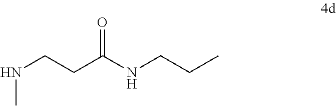

3-[(tert-butoxycarbonyl)(methyl)amino]propanoic acid (102 mg, 0.50 mmol) was dissolved in DMF (0.5 mL). 1-propylamine (49 μL, 0.59 mmol), PyBOP (286 mg, 0.55 mmol) and DIPEA (171 μL, 0.98 mmol). After 3 h the reaction was quenched with TFA (50 μl) and the product purified by RP-HPLC. The product was dissolved in 0.5 ml of DCM. 0.5 mL of TFA were added with stirring in an open flask. After 1 h the reaction was concentrated in a stream of nitrogen and the product co-evaporated 2 times with DCM. The residue was dissolved in water (2 mL) and lyophilized.

Yield: 128 mg (99%, TFA salt)

MS: m/z 144.92=[M+H]⁺, (calculated=145.14).

### Example 8: Synthesis of Compound 4e

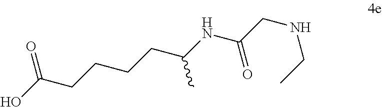 [SMILES: CCNCC(=O)NC(C)CCCCC(=O)O]

Methyl 6-oxo-heptanoate (2 g, 12.64 mmol) was dissolved in methanol (13 mL) and ammonium acetate (9.75 g, 126.43 mmol), and sodium cyanoborohydride (1.19 g, 18.96 mmol) was added with stirring. The resulting suspension turned into a solution and stirring was continued overnight. The mixture was diluted with water (70 mL) and ethyl acetate was added (80 mL). The pH of the water phase was adjusted to ca pH 11 with 25 mL 4 M NaOH. The water phase was extracted 3 more times with 70 mL ethyl acetate. The combined organic phases were dried (MgSO₄), filtered and concentrated in vacuo. The crude from the first step was dissolved in DMF (20 mL) and N-Boc-N-ethylglycine (2.55 g, 12.56 mmol), PyBOP (7.19 g, 13.82 mmol) and DIPEA (6.56 mL, 37.68 mmol) were added with stirring. After 1 h the reaction was diluted with 60 mL ethyl acetate and washed with 0.1 M HCl (3 times 80 mL), 0.5 M NaOH (3 times 50 mL) and brine (50 mL). The organic phase was dried (MgSO₄), filtered and concentrated in vacuo. The residue was purified using flash chromatography (heptane/ethyl acetate). The product was dissolved in THF (10 mL), and LiOH (0.46 g, 19.21 mmol) was dissolved in water (4 mL). The solutions were combined and stirred vigorously. After 3 h the reaction was diluted with 80 mL ethyl acetate and 60 mL 1 M HCl was added. The pH of the aqueous phase was below 2. The organic phase was collected and the aqueous phase extracted with ethyl acetate (2 times 50 mL). The combined organic solution was dried (MgSO₄), filtered and concentrated in vacuo. The residue was dissolved in DCM (10 mL) and TFA (5 mL) was added with vigorous stirring in an open flask. After 30 min the reaction was concentrated in vacuo and co-evaporated once with 5 mL DCM. The crude was dissolved in water (40 mL) and lyophilized.

Yield: 2.54 g (59%, TFA salt)

MS: m/z 230.94=[M+H]⁺, (calculated=231.17).

### Example 9: Synthesis of Compound 4f

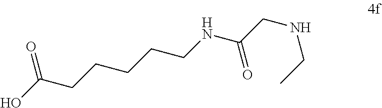

Compound 4f was synthesized using solid phase synthesis following the general protocol using Fmoc-Ahx-OH and Boc-N-ethyl glycine as building blocks. Upon cleavage from the resin the BOC protecting group was removed concurrently using the TFA cleavage cocktail. The cleavage solution was concentrated in vacuo, and the residue was dissolved in acetonitrile/water and lyophilized.

Yield: 1.01 g (quant., TFA salt)

MS: m/z 216.92=[M+H]⁺, (calculated=217.16).

### Example 10: Synthesis of Compound 4g

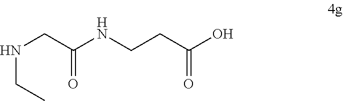

N-Boc-N-ethylglycine (100 mg, 0.49 mmol) and HOBt (66 mg, 0.49 mmol) were suspended in DCM (1 mL). H-beta-Ala-OtBu hydrochloride (107 mg, 0.59 mmol) was added and a solution was obtained. EDC HCl (99 mg, 0.52 mmol) was added and the reaction was stirred for 1.5 h. The volatiles were removed in vacuo and the product purified by RP-HPLC. The product was dissolved in 0.5 mL of DCM. 0.5 mL of TFA were added with stirring in an open flask. After 30 min the reaction was concentrated in vacuo and the product co-evaporated 2 times with DCM. The residue was dissolved in acetonitrile/water 1:1 (2 mL) and lyophilized.

Yield: 125 mg (88%, TFA salt)

MS: m/z 174.98=[M+H]⁺, (calculated=175.11).

### Example 11: Synthesis of Compound 4h

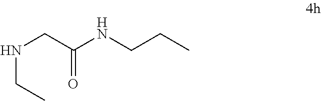

N-Boc-N-ethylglycine (102 mg, 0.50 mmol) was dissolved in DMF (0.5 mL). 1-propylamine, (49 μL, 0.59 mmol), PyBOP (281 mg, 0.54 mmol) and DIPEA (171 μL, 0.98 mmol) were added. After 3.5 h TFA (40 μL) was added and the product purified by RP-HPLC. The product was dissolved in 0.5 ml of DCM. 0.5 ml of TFA were added with stirring in an open flask. After 1 h the reaction was concentrated in vacuo and the product co-evaporated 2 times with DCM (2 mL).

Yield: 122 mg (94%, TFA salt)

MS: m/z 144.89=[M+H]⁺, (calculated=145.14).

### Example 12: Synthesis of Compound 4i

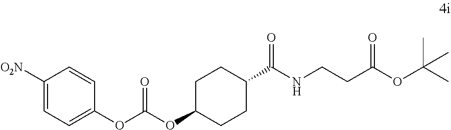 [SMILES: CC(C)(C)OC(=O)CCNC(=O)[C@H]1CC[C@H](OC(=O)Oc2ccc([N+](=O)[O-])cc2)CC1]

To a suspension of trans-4-hydroxycyclohexanoic acid (61 mg, 0.43 mmol) in DCM (0.8 mL) was added HOBt (63 mg, 0.47 mmol) then DIC (73 μL, 0.47 mmol). To the suspension was added DMF (0.2 mL). H-beta-Ala-OtBu hydrochloride (86 mg, 0.47 mmol) in DCM (0.2 mL). After 4.5 h DIPEA (60 μL) was added. After 5 h the reaction was diluted with DCM (ca. 10 mL) and filtered. The filtrate was washed with aq. 0.1 M HCl, then with brine. The organic phase was dried over MgSO₄, filtered and concentrated in vacuo. The product was purified by RP-HPLC. The product (57.00 mg, 0.21 mmol) was dissolved in DCM (2.5 mL) and DMAP (26 mg, 0.21 mmol) was added. 4-nitrophenyl chloroformate (85 mg, 0.42 mmol) in DCM (0.5 mL) and DIPEA (110 μL, 0.63 mmol) were added. After 1 h 0.1 M aq. HCl (15 mL) was added and the mixture diluted with ethyl acetate (30 mL). The organic phase was washed with 0.1 M aq. HCl (2 times 10 mL). The aq phase was re-extracted with ethyl acetate (3 times 10 mL). The organic phases were combined, dried over MgSO₄, filtered, and concentrated in vacuo.

Yield: 119 mg (65%)

MS: m/z 437.21=[M+H]⁺, (calculated=437.19).

### Example 13: Synthesis of Compound 4j

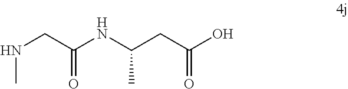

Boc-Sar-OH (103 mg, 0.54 mmol) was dissolved in DCM (1 mL). tert-Butyl-(3S)-3-aminobutanoate (84 mg, 0.53 mmol), EDC HCl (113 mg, 0.59 mmol) and DIPEA (0.28 mL, 1.58 mmol) were added. After 3 h the reaction mixture was diluted with 30 mL of DCM and was washed 3 times with 30 mL of 0.1 N HCl, 2 times with sat. NaHCO₃ and once with brine. The organic phase was dried over Na₂SO₄, filtered and evaporated. The product was purified by RP-HPLC. The product was dissolved in 0.5 mL of DCM. 0.5 ml of TFA were added with stirring in an open flask. After 3 h the reaction was concentrated in vacuo and the product co-evaporated 3 times with DCM (5 mL).

Yield: 73 mg (47%)

MS: m/z 437.21=[M+H]⁺, (calculated=437.19).

### Example 14: Synthesis of Compound 5a

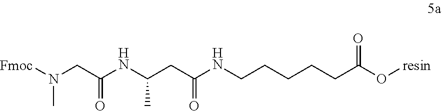

5a was synthesized using solid phase synthesis following the general protocol using Fmoc-Ahx-OH, Fmoc-beta homoalanine-OH and Fmoc-Sar-OH as building blocks.

### Example 15: Synthesis of Compound 5b

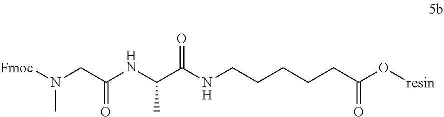

5b was synthesized using solid phase synthesis following the general protocol using Fmoc-Ahx-OH, Fmoc-Ala-OH and Fmoc-Sar-OH as building blocks.

### Example 16: Synthesis of Compound 5c

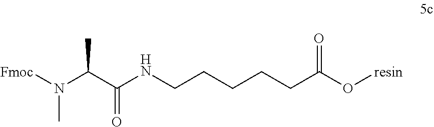

5c was synthesized using solid phase synthesis following the general protocol using Fmoc-Ahx-OH, and Fmoc-N-Methyl-Ala-OH as building blocks.

### Example 17: Synthesis of Compound 5d

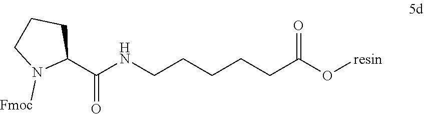

5c was synthesized using solid phase synthesis following the general protocol using Fmoc-Ahx-OH, and Fmoc-Pro-OH as building blocks.

### Example 18: Synthesis of Compound 6a

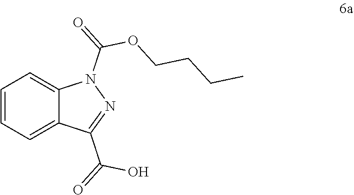 [SMILES: CCCCOC(=O)n1nc(C(=O)O)c2ccccc21]

1H-indazole-3-carboxylic acid (40 mg, 0.25 mmol) was dissolved in DMF (0.5 mL). DIPEA (172 μL, 0.99 mmol) and n-butyl chloroformate (63 μL, 0.49 mmol) were added. The mixture was stirred for 2.5 h. TFA (95 μL) was added and the product was purified by RP-HPLC.

Yield: 0.8 mg (1%)

MS: m/z 262.91=[M+H]⁺, (calculated=263.11).

### Example 19: Synthesis of Compound 6b

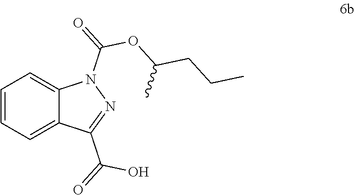 [SMILES: CCCC(C)OC(=O)n1nc(C(=O)O)c2ccccc21]

4-nitrophenyl chloroformate (50 mg, 0.25 mmol) was dissolved in DCM (0.50 mL). To the stirred reaction 2-pentanol (25 μL, 0.23 mmol) and TEA (79 μL, 0.57 mmol) were added. After 4 h volatiles were removed in a stream of nitrogen and the product purified by RP-HPLC. The product (17 mg, 0.07 mmol) was dissolved in 0.5 mL acetonitrile and 1H-indazole-3-carboxylic acid (10 mg, 0.06 mmol) was added. DIPEA (27 μL, 0.15 mmol) was added. After 2 h again DIPEA (27 μL, 0.15 mmol) was added. After 4 h acetic acid (100 μL) was added and the product purified by RP-HPLC.

Yield: 15 mg (53%)

MS: m/z 277.12=[M+H]⁺, (calculated=277.12).

### Example 20: Synthesis of Compound 6c

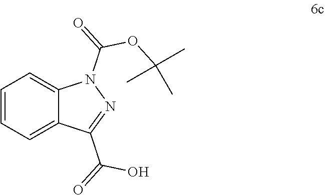 [SMILES: CC(C)(C)OC(=O)n1nc(C(=O)O)c2ccccc21]

1H-indazole-3-carboxylic acid (20 mg, 0.12 mmol) and di-tert-butyl dicarbonate (30 mg, 0.14 mmol) were suspended in acetonitrile (0.50 mL). DMAP (1.5 mg, 0.01 mmol) and DIPEA (32 μL, 0.19 mmol) were added and the reaction stirred for 2.25 h. The product was purified by RP-HPLC.

Yield: 18 mg (56%)

MS: m/z 547.18=[2M+Na]⁺, (calculated=547.18).

### Example 21: Synthesis of Compound 6d

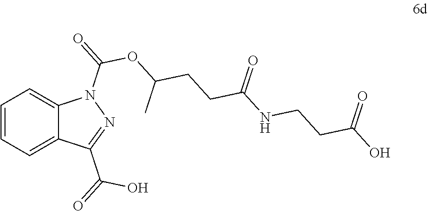 [SMILES: CC(CCC(=O)NCCC(=O)O)OC(=O)n1nc(C(=O)O)c2ccccc21]

H-beta-Ala-OtBu hydrochloride (245 mg, 1.35 mmol) was dissolved in DMF (3 mL) and cooled under stirring in an ice-bath for 10 min before sequential addition of DIPEA (250 μL, 1.44 mmol), gamma-valerolactone (86 μL, 0.90 mmol) and tin(II)acetate (46 mg, 0.20 mmol). After 5 more minutes of cooling, the solution was heated to 80° C. with stirring for 6.75 h. The reaction was diluted with ethyl acetate (25 mL), washed with 0.1 M aq. HCl (30 mL) and brine (2 times 25 mL). The combined organic phases were dried (MgSO₄), filtered and concentrated in vacuo. The product was purified by RP-HPLC.

The aforementioned product (4 mg, 16 μmol) was dissolved in DCM (0.2 mL) and 4-nitrophenyl chloroformate (6.5 mg, 32 μmol) in DCM (50 μL) was added. DIPEA (8 μL, 46 μmol) and DMAP (1.9 mg, 16 μmol) were added. After 2 h the reaction was quenched by addition of 0.1 M aq. HCl (2 mL) and diluted with ethyl acetate (ca. 2 mL). The organic phase was extracted, and then the aq. phase re-extracted with ethyl acetate (4 times ca. 2 mL). The organics were combined and dried over MgSO₄, filtered, and the volatiles removed in vacuo. The activated PNP carbonate from the previous step was used without further purification. 1H-indazole-3-carboxylic acid (3.2 mg, 20 μmol) in DCM (0.4 ml) was added. DIPEA (9 μL, 52 μmol) was added and the reaction stirred overnight. The product was purified by RP-HPLC. The product from the former step was dissolved in DCM (0.4 mL) and TFA (0.2 mL) was added. After 1 h volatiles were removed in vacuo and the residue dissolved in water and lyophilized.

Yield: 1.7 mg (0.6%)

MS: m/z 378.06=[M+H]⁺, (calculated=378.13).

### Example 22: Synthesis of Compound 6e

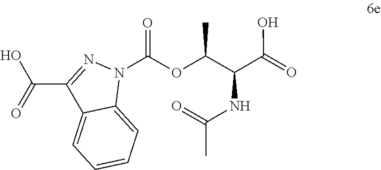 [SMILES: CC(=O)N[C@H](C(=O)O)[C@H](C)OC(=O)n1nc(C(=O)O)c2ccccc21]

H-Thr-OtBu (59 mg, 0.28 mmol) was dissolved in DMF (0.5 mL), and DIPEA (145 μL, 0.84 mmol) was added. After 5 min, N-acetoxysuccinimide (54 mg, 0.34 mmol) in DMF (0.2 mL) was added drop-wise. After 40 min the reaction was diluted with ethyl acetate (15 mL) then washed sequentially with 0.1 M aq. HCl (3 times 10 mL), sat. NaHCO₃ (2 times 10 mL) and brine (2 times 10 mL). The organic phase was dried over MgSO₄, filtered, and concentrated in vacuo. The product was purified by RP-HPLC.

To a stirred solution of the product from the former step (4.6 mg, 21 μmol) in DCM (0.2 mL) was added 4-nitrophenyl chloroformate (8.7 mg, 43 μmol) in DCM (0.1 ml). Under stirring DIPEA (11 IL, 63 μmol) and DMAP (2.7 mg, 22 μmol) were added. After 2.25 h the reaction was diluted with ethyl acetate (ca. 2 mL) then washed with 0.1 M aq. HCl (2 mL). Re-extracted from the aq. phase with ethyl acetate (3 times ca. 3 mL). The organics were combined, dried over MgSO₄, filtered and concentrated in vacuo.

The activated PNP carbonate from the previous step was used without further purification. 1H-indazole-3-carboxylic acid (4.7 mg, 29 μmol) in DCM (0.4 mL) was added. DIPEA (11 IL, 63 μmol) was added. After stirring overnight, the volatiles were removed in vacuo and the product was purified by RP-HPLC. The product from the former step was dissolved in DCM (0.4 mL) and TFA (0.2 mL) was added. After 4 h volatiles were removed in vacuo and the residue dissolved in water and lyophilized.

Yield: 1.1 mg (11.2%, TFA salt)

MS: m/z 350.06=[M+H]⁺, (calculated=350.10).

### Example 23: Synthesis of Compound 6f

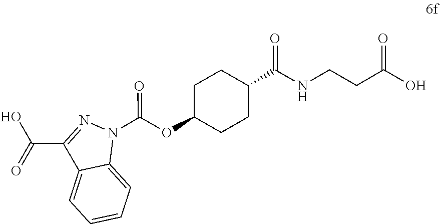 [SMILES: O=C(O)CCNC(=O)[C@H]1CC[C@H](OC(=O)n2nc(C(=O)O)c3ccccc32)CC1]

4i (22.9 mg, 52 μmol) and 1H-indazole-3-carboxylic acid (7.5 mg, 46 μmol) were suspended in DCM (0.55 mL). DIPEA (25 μL, 144 μmol) was added and the reaction stirred overnight. More DIPEA (3 times 8.5 μl) was added after 1 h, 1.5 h, 3.5 h. Next day the reaction was diluted with ethyl acetate (25 mL) and washed with 0.1 M aq. HCl (2 times 10 mL) and brine (10 mL). The organic phase was dried over Na₂SO₄, filtered and concentrated in vacuo. The product was purified by RP-HPLC. The product from the former step (2.3 mg) was dissolved in DCM (0.1 mL) and TFA (0.1 mL) was added. After 2.25 h volatiles were removed in vacuo and the residue dissolved in water and lyophilized.

Yield: 1.6 mg (38%)

MS: m/z 460.15=[M+H]⁺, (calculated=460.21).

### Example 24

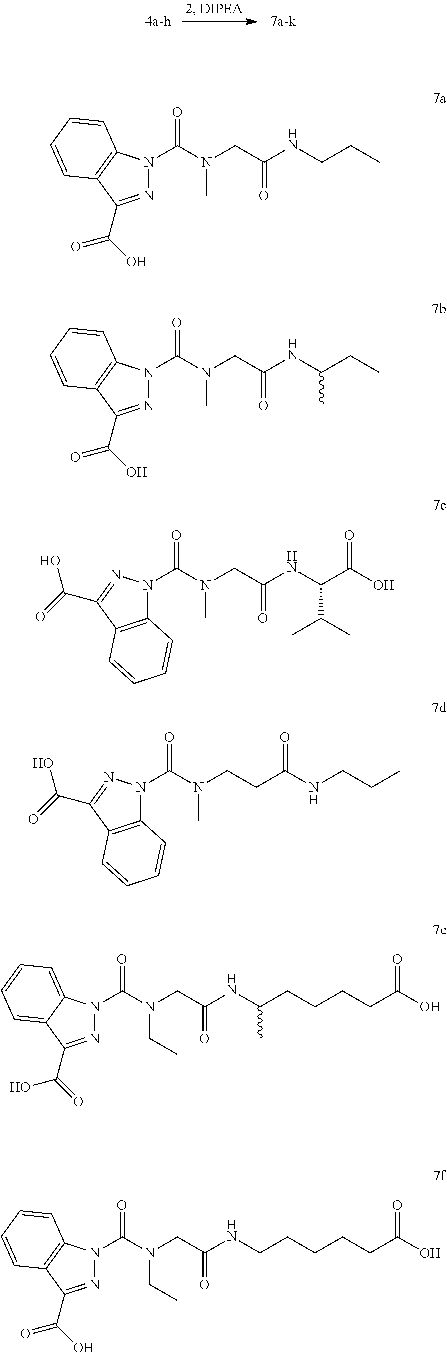

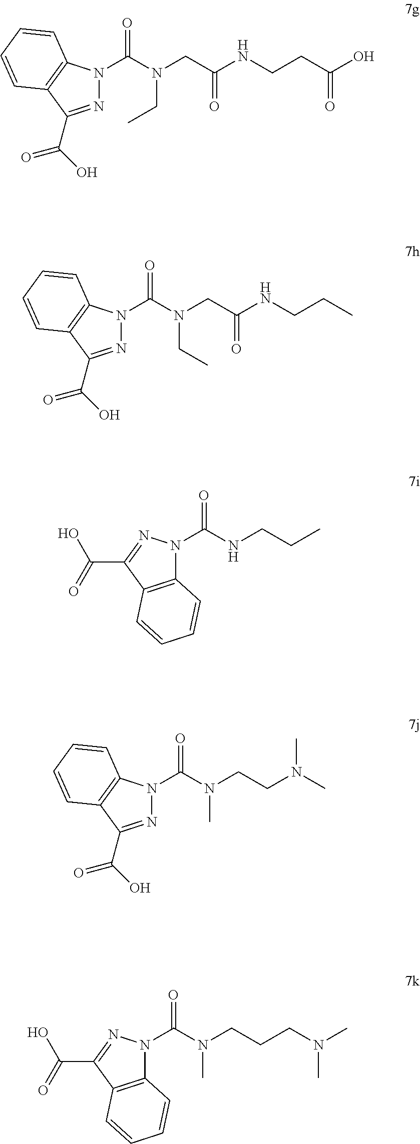 [SMILES: CCN(CC(=O)NCCC(=O)O)C(=O)n1nc(C(=O)O)c2ccccc21 ; CCCNC(=O)CN(CC)C(=O)n1nc(C(=O)O)c2ccccc21 ; CCCNC(=O)n1nc(C(=O)O)c2ccccc21 ; CN(C)CCN(C)C(=O)n1nc(C(=O)O)c2ccccc21 ; CN(C)CCCN(C)C(=O)n1nc(C(=O)O)c2ccccc21]

Conjugates 7a-k were synthesized by reacting 1 eq. of 2 with 1.0-1.2 eq. of the respective amine 4a-h or for 7i: 1-propylamine, 7j: N,N,N′-trimethylethylene, 7k: N,N,N′-trimethyl-1,3-propane diamine using excess DIPEA in DMF. Reactions were quenched using excess TFA and purified by RP-HPLC.

7a: 2: 19 mg, 59 μmol, 4a: 9 mg, 69 μmol, DIPEA: 21 μL, 0.12 mmol:

Yield: 2.1 mg (11%), MS: m/z 319.09=[M+H]⁺, (calculated=319.14).

7b: 2: 20 mg, 60 μmol, 4b: 12 mg, 69 μmol, DIPEA: 21 μL, 0.12 mmol:

Yield: 5.5 mg (27%), MS: m/z 333.11=[M+H]⁺, (calculated=333.16).

7c: 2: 10 mg, 31 μmol, 4c: 11 mg, 35 μmol, DIPEA: 21 μL, 0.12 mmol:

Yield: 2.3 mg (19%), MS: m/z 377.06=[M+H]⁺, (calculated=377.15).

7d: 2: 21 mg, 65 μmol, 4d: 18 mg, 66 μmol, DIPEA: 43 μL, 0.25 mmol:

Yield: 6.5 mg (30%), MS: m/z 333.11=[M+H]⁺, (calculated=333.16).

7e: 2: 17 mg, 51 μmol, 4e: 17 mg, 49 μmol, DIPEA: 21 μL, 0.12 mmol:

Yield: 3.9 mg (18%), MS: m/z 419.17=[M+H]⁺, (calculated=419.20).

7f: 2: 10 mg, 31 μmol, 4f: 11 mg, 34 μmol, DIPEA: 21 μL, 0.12 mmol:

Yield: 1.7 mg (14%), MS: m/z 405.03=[M+H]⁺, (calculated=405.18).

7g: 2: 20 mg, 60 μmol, 4g: 24 mg, 69 μmol, DIPEA: 21 μL, 0.12 mmol:

Yield: 1.9 mg (9%), MS: m/z 363.03=[M+H]⁺, (calculated=363.13).

7h: 2: 20 mg, 60 μmol, 4h: 24 mg, 68 μmol, DIPEA: 43 μL, 0.25 mmol:

Yield: 4 mg (20%), MS: m/z 333.18=[M+H]⁺, (calculated=333.16).

7i: 2: 22 mg, 66 μmol, 4i: 5 μl, 61 μmol, DIPEA: 21 μL, 0.12 mmol:

Yield: 4.7 mg (29%), MS: m/z 247.96=[M+H]⁺, (calculated=248.11).

7j: 2: 16 mg, 50 μmol, 4j: 8 μl, 62 μmol, DIPEA: 21 μL, 0.12 mmol:

Yield: 8 mg (52%), MS: m/z 291.03=[M+H]⁺, (calculated=291.15).

7k: 2: 21 mg, 65 μmol, 4k: 9.4 μl, 64 μmol, DIPEA: 21 μL, 0.12 mmol:

Yield: 0.4 mg (1%), MS: m/z 305.06=[M+H]⁺, (calculated=305.16).

### Example 25

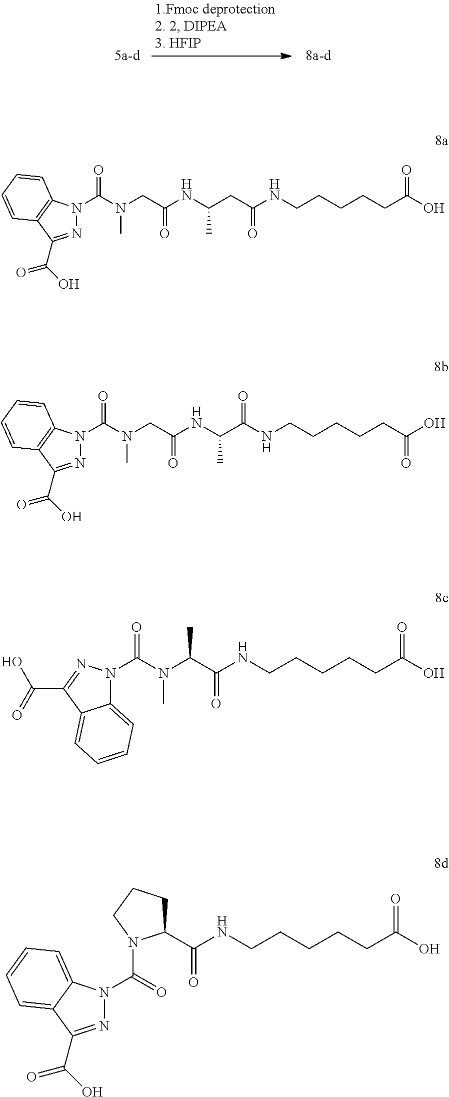 [SMILES: C[C@@H](CC(=O)NCCCCCC(=O)O)NC(=O)CN(C)C(=O)n1nc(C(=O)O)c2ccccc21 ; C[C@H](NC(=O)CN(C)C(=O)n1nc(C(=O)O)c2ccccc21)C(=O)NCCCCCC(=O)O ; C[C@@H](C(=O)NCCCCCC(=O)O)N(C)C(=O)n1nc(C(=O)O)c2ccccc21 ; O=C(O)CCCCCNC(=O)[C@@H]1CCCN1C(=O)n1nc(C(=O)O)c2ccccc21]

Conjugates 8a-e were synthesized by Fmoc deprotection of 5a-d using 2:2:96 piperidine/DBU/DMF, following reacting an excess of 2 with the respective amine on resin using excess DIPEA in DMF. The product was cleaved from the resin using HFIP and purified by RP-HPLC.

8a: 5a: 7 mg, 5 μmol, 2: 5 mg, 16 μmol, DIPEA: 5 μL, 31 μmol

Yield: 1.1 mg (45%), MS: m/z 476.02=[M+H]⁺, (calculated=476.22).

8b: 5b: 7 mg, 5 μmol, 2: 5 mg, 15 μmol, DIPEA: 5 μl, 31 μmol

Yield: 1.3 mg (55%), MS: m/z 462.12=[M+H]⁺, (calculated=462.20).

8c: 5c: 50 μmol, 2: 44 mg, 0.13 mmol, DIPEA: 50 μl, 0.29 mmol

Yield: 6.5 mg (32%), MS: m/z 405.16=[M+H]⁺, (calculated=405.18).

8d: 5d: 55 μmol, 2: 52 mg, 0.16 mmol, DIPEA: 55 μl, 0.32 mmol

Yield: 11 mg (48%), MS: m/z 417.16=[M+H]⁺, (calculated=417.18).

### Example 26: Synthesis of Compound 9a

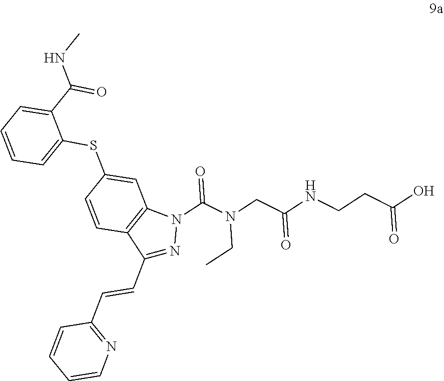 [SMILES: CCN(CC(=O)NCCC(=O)O)C(=O)n1nc(/C=C/c2ccccn2)c2ccc(Sc3ccccc3C(=O)NC)cc21]

4g (29 mg, 82 μmol) was dissolved in 100 μL of DMF and DIPEA (48 μL, 0.27 mmol) was added. A suspension of 3 (40 mg, 68 μmol) (0.79 mL in DMF) was added. After 3.5 h 4g (14 mg, 41 μmol) in 50 μL of DMF was added. After 4.75 h TFA (21 μL) were added and the reaction purified by RP-HPLC.

Yield: 22 mg (45%, TFA salt)

MS: m/z 350.06=[M+H]⁺, (calculated=350.10).

### Example 27: Synthesis of Compound 9b

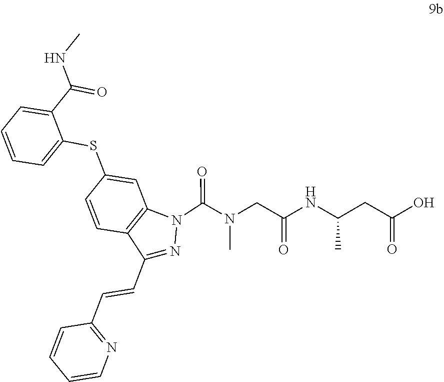 [SMILES: CNC(=O)c1ccccc1Sc1ccc2c(/C=C/c3ccccn3)nn(C(=O)N(C)CC(=O)N[C@@H](C)CC(=O)O)c2c1]

4j (28 mg, 88 μmol) was dissolved in 100 μL of DMF and DIPEA (38 μL, 0.22 mmol) was added. A suspension of 3 (26 mg, 44 μmol) (508 μL in DMF) was added. After 30 min TFA (6.7 μL) was added and the product purified by RP-HPLC.

Yield: 31 mg (quant, TFA salt)

MS: m/z 587.16=[M+H]⁺, (calculated=587.21).

### Example 28: Synthesis of Compound 9c

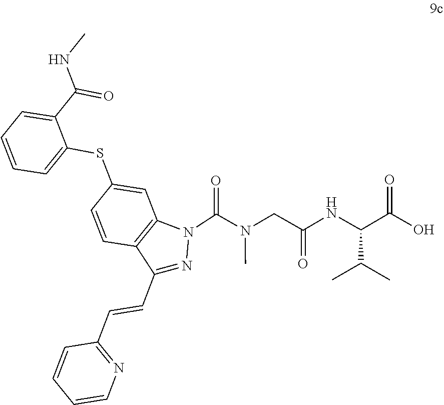 [SMILES: CNC(=O)c1ccccc1Sc1ccc2c(/C=C/c3ccccn3)nn(C(=O)N(C)CC(=O)N[C@H](C(=O)O)C(C)C)c2c1]

4c (22 mg, 68 μmol) was dissolved in 100 μL of DMF and DIPEA (30 μL, 0.17 mmol) was added. A suspension of 3 (20 mg, 34 μmol) (393 μL in DMF) was added. After 1 h TFA (5.2 μl) was added and the product purified by RP-HPLC.

Yield: 26 mg (quant, TFA salt)

MS: m/z 601.10=[M+H]⁺, (calculated=601.23).

### Example 29: Synthesis of Compounds 9d and 9e

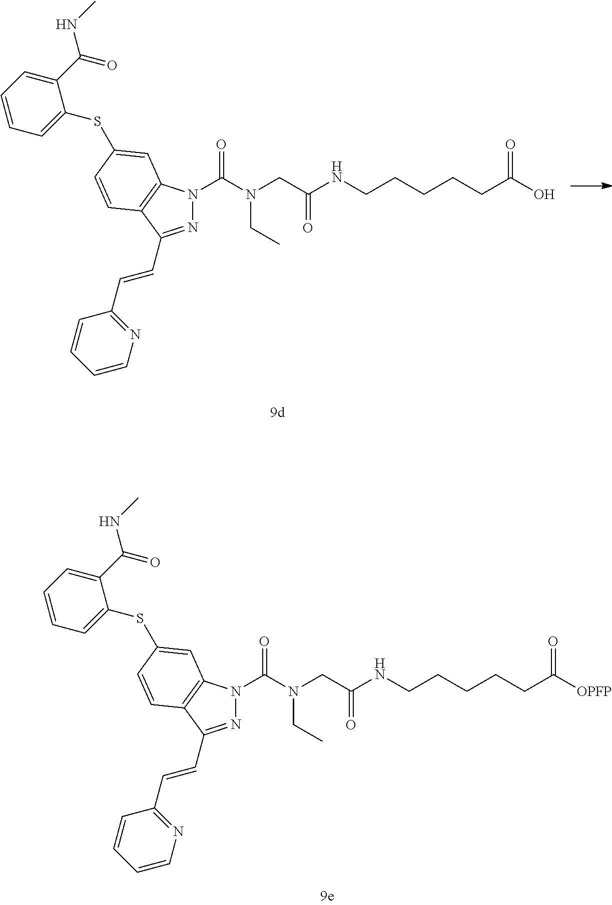

4f (689 mg, 2.09 mmol) was dissolved in 4 mL of DMF and DIPEA (0.9 mL, 5.2 mmol) was added. A suspension of 3 (0.61 g, 0.98 mmol) (8.2 mL in DMF) was added. After 30 min the reaction was added to a solution of 2.6 mL 4 N HCl in dioxane and 237 mL of ethyl acetate. The precipitate was centrifuged, the supernatant decanted and the residue washed once with 180 mL ethyl acetate. The residue was purified by RP-LPLC to obtain compound 9d.

Yield: 0.34 g (46%, TFA salt)

MS: m/z 629.34=[M+H]⁺, (calculated=629.26).

9d (0.34 g; 0.45 mmol) was dissolved in DMF (6.76 mL) and bis(pentafluorophenyl) carbonate (0.21 g, 0.54 mmol) was added. DIPEA (0.48 mL, 2.73 mmol) was added. After 45 min acetic acid (0.48 mL) was added and the product purified by RP-LPLC to obtain compound 9e.

Yield: 0.40 g (98%, TFA salt)

MS: m/z 795.39=[M+H]⁺, (calculated=795.24).

### Example 30: Synthesis of Compound 9f

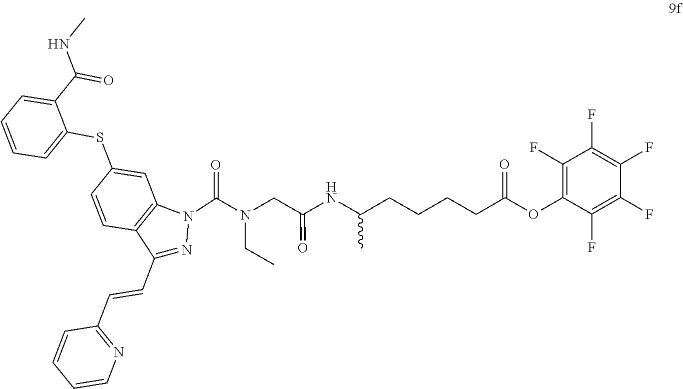 [SMILES: CCN(CC(=O)NC(C)CCCCC(=O)Oc1c(F)c(F)c(F)c(F)c1F)C(=O)n1nc(/C=C/c2ccccn2)c2ccc(Sc3ccccc3C(=O)NC)cc21]

A suspension of 3 (8.50 mL, 0.13 mol/L; 1.04 mmol) in DMF was added to 4e (0.72 g, 2.08 mmol) and DIPEA (0.91 mL, 5.21 mmol) was added. After 45 min the reaction was added to a solution of 2.6 mL 4 N HCl in dioxane and 160 mL of ethyl acetate. The precipitate was centrifuged, the supernatant decanted and the residue purified by RP-LPLC.

The product from the former step (0.42 g, 0.55 mmol) was dissolved in DMF (8.40 mL) and bis(pentafluorophenyl) carbonate (0.27 g, 0.67 mmol) was added. DIPEA (0.58 mL, 3.33 mmol) was added. After 1 h acetic acid (0.48 mL) was added and the product purified by RP-LPLC.

Yield: 0.27 g (28%, TFA salt)

MS: m/z 809.36=[M+H]⁺, (calculated=809.26).

### Example 31: Synthesis of Compound 9g

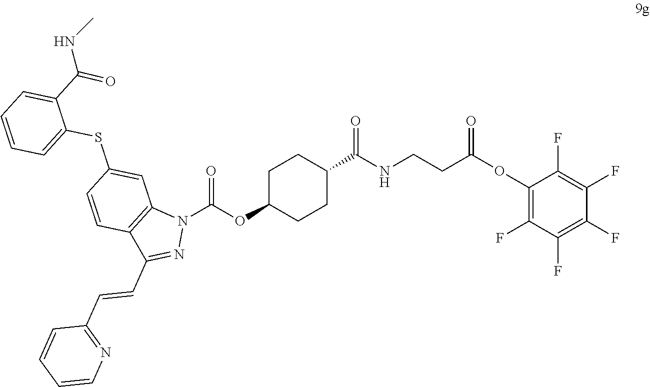 [SMILES: CNC(=O)c1ccccc1Sc1ccc2c(/C=C/c3ccccn3)nn(C(=O)O[C@H]3CC[C@H](C(=O)NCCC(=O)Oc4c(F)c(F)c(F)c(F)c4F)CC3)c2c1]

A solution of 4i (49 mg, 0.11 mmol) in THF (1.60 mL) was added to axitinib (22 mg, 56 μmol). DIPEA (49 μL, 0.28 mmol) was added. The reaction was heated to 60° C. for 6 h and stirred overnight at RT. DMF (0.5 ml) was added. The reaction was heated to 60° C. for 6.5 h and stirred for 3 days at RT. DMAP (>1 eq.) was added and the reaction stirred at RT for 1 day. TFA (25 μL) was added and the product purified by RP-HPLC.

The product from the former step (16 mg, 20 μmol) was dissolved in a mixture of DCM (1 mL) and TFA (1 mL). After 2 h the volatiles were removed in vacuo and the residue dissolved in 3 mL acetonitrile/water/TFA 1:1:0.002 and lyophilized.

The product from the former step (15 mg, 20 μmol) was dissolved in DMF (0.29 mL) and bis(pentafluorophenyl) carbonate (9.4 mg 24 μmol) was added. DIPEA (21 μL; 0.12 mmol) was added. After 1.5 h TFA (10 μL) was added and the reaction purified by RP-HPLC.

Yield: 13 mg (25%, TFA salt)

MS: m/z 794.25=[M+H]⁺, (calculated=794.21).

### Example 32: Synthesis of Compounds 10a-d

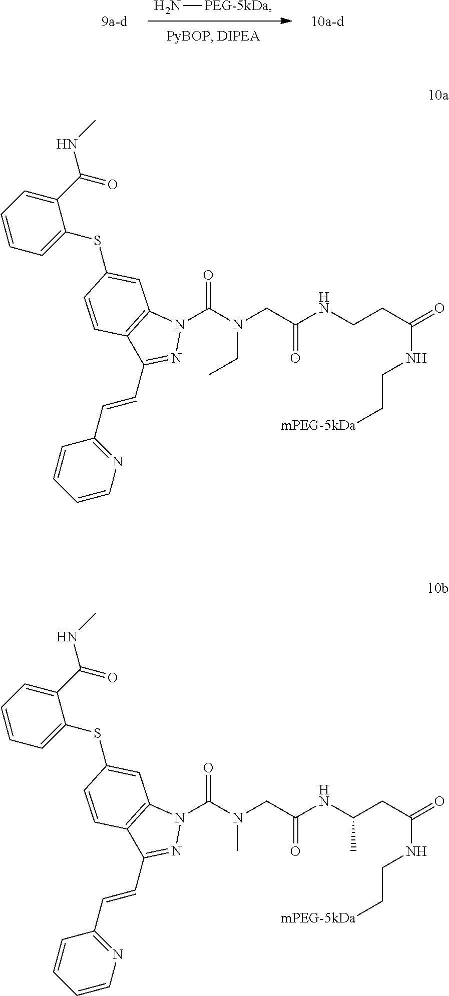

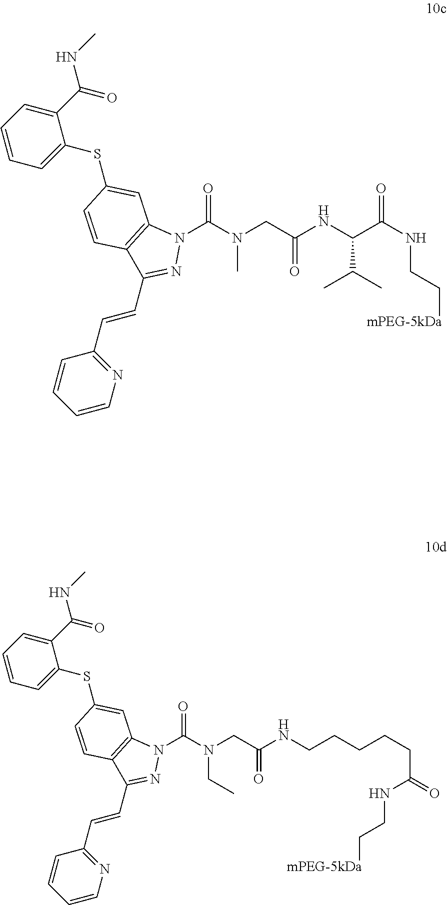

Methoxy polyethylene glycol amine-5 kDa PEG, PyBOP, DIPEA and 9 were stirred at RT. After the reaction was finished, acetic acid was added, and the product purified by RP-HPLC.

10a: PEG: 33 mg, 6.0 μmol, PyBOP: 3.6 mg, 6.9 μmol, DIPEA: 3.1 μl, 18 μmol, 9a: 4.2 mg, 6 μmol, yield: 21 mg (58%, TFA salt).

10b: PEG: 21 mg, 3.8 μmol, PyBOP: 3.5 mg, 6.7 μmol, DIPEA: 1.9 μl, 11 μmol, 9b: 2.6 mg, 3.7 μmol, yield: 18 mg (77%, TFA salt).

10c: PEG: 47 mg, 8.5 μmol, PyBOP: 4.9 mg, 9.4 μmol, DIPEA: 4.4 μl, 25 μmol, 9c: 6 mg, 8.4 μmol, yield: 31 mg (60%, TFA salt).

10d: PEG: 31 mg, 5.6 μmol, PyBOP: 3.5 mg, 6.8 μmol, DIPEA: 2.8 μL, 16 μmol, 9d: 4 mg, 5.4 μmol, yield: 34 mg (quant, TFA salt).

### Example 33: Synthesis of Compounds 11a-d

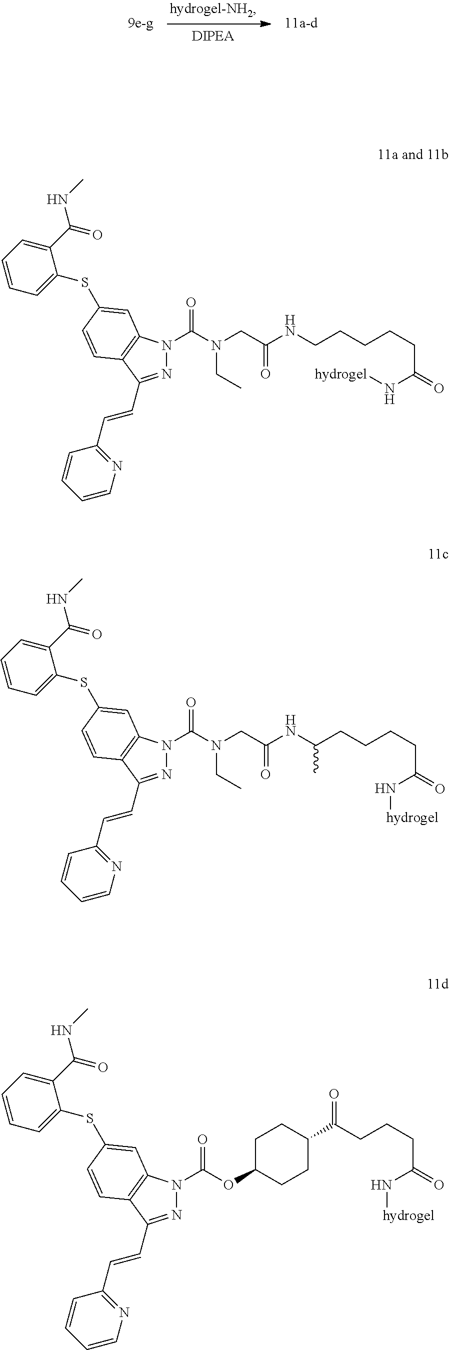

The hydrogel was swollen in 1% DIPEA in DMF in a syringe reactor containing a PE frit. The syringe reactor was 3 times filled, shaken for 1 min and drained. 9 was dissolved in DMF and DIPEA was added. The solution was drawn into the syringe containing the hydrogel. The syringe was shaken for longer than 16 h at RT. The syringe was drained, and the hydrogel was washed several times with DMF and ethanol and dried in vacuo or washed several times with DMF, water and pH 5.5 20 mM sodium succinate aqueous buffer and a hydrogel suspension in pH 5.5 aqueous buffer was obtained.

11a: HG-1: 14 mg, DIPEA: 1.6 μL, 9e: 3 mg,

yield: 15 mg, dried 39 mg/g axitinib in dried hydrogel.

11b: HG-2: 0.82 g, DIPEA: 0.21 mL, 9e: 0.40 g

yield: suspension, 7.55 mg/mL axitinib in hydrogel suspension.

11c: HG-3: 30 mg, DIPEA: 3.5 μL, 9f: 6.7 mg,

yield: suspension, 2.93 mg/mL axitinib in hydrogel suspension.

11d: HG-3: 30 mg, DIPEA: 3.5 μL, 9g: 13 mg

yield: suspension, 3.65 mg/mL axitinib in hydrogel suspension.

### Example 34: In Vitro Release Kinetics

The cleavage rate of the reversible bond from conjugates 6a-f, 7a-k, 8a-d, 10a-d, 11a-d was monitored at pH 7.4 and 37° C. in aqueous buffer (pH 7.4 48 mM sodium phosphate, 20% acetonitrile or pH 7.4 60 mM sodium phosphate). For soluble examples disappearance of the conjugate was determined by LCMS (UV detection) and fitted with curve fitting software to obtain the preliminary half-life of the release. For insoluble examples (hydrogels) the increase in released heteroaromatic moiety containing molecule in the supernatant was determined by LCMS (UV detection) and used as input for the curve fitting software to obtain the preliminary half-life of the release.

| Compound | t_{1/2} (pH 7.4) | Released | product |
|---|---|---|---|
| 1 h** | <1 min | indazole | |
| 6a* | 21 d | 1H-indazole-3-carboxylic | acid |
| 6b* | 99 d | 1H-indazole-3-carboxylic | acid |
| 6c* | 2 h | 1H-indazole-3-carboxylic | acid |
| 6d* | 36 d | 1H-indazole-3-carboxylic | acid |
| 6e* | 1 d | 1H-indazole-3-carboxylic | acid |
| 6f* | 39 d | 1H-indazole-3-carboxylic | acid |
| 7a* | 3.5 d | 1H-indazole-3-carboxylic | acid |
| 7b* | 17 d | 1H-indazole-3-carboxylic | acid |
| 7c* | >180 d | 1H-indazole-3-carboxylic | acid |
| 7d* | >180 d | 1H-indazole-3-carboxylic | acid |
| 7e* | 52 d | 1H-indazole-3-carboxylic | acid |
| 7f* | 9.6 d | 1H-indazole-3-carboxylic | acid |
| 7g* | 7.7 d | 1H-indazole-3-carboxylic | acid |
| 7h* | 8.1 d | 1H-indazole-3-carboxylic | acid |
| 7i* | >180 d | 1H-indazole-3-carboxylic | acid |
| 7j* | 2.9 h | 1H-indazole-3-carboxylic | acid |
| 7k* | 164 d | 1H-indazole-3-carboxylic | acid |
| 8a* | 2.4 d | 1H-indazole-3-carboxylic | acid |
| 8b* | 8.2 h | 1H-indazole-3-carboxylic | acid |
| 8c* | 13 d | 1H-indazole-3-carboxylic | acid |
| 8d* | 12 d | 1H-indazole-3-carboxylic | acid |
| 10a | 14 h | axitinib | |
| 10b | 19 h | axitinib | |
| 10c | 8 h | axitinib | |
| 10d | 1.9 d | axitinib | |
| 11a | 3.7 d | axitinib | |
| 11b | 5.4 d | axitinib | |
| 11c | 17.4 d | axitinib | |
| 11d | 62 d | axitinib | |

The compounds marked with “*” and “**” are not in accordance with the present invention as they were for efficiency reasons not linked to a moiety Z. Nevertheless, they show the release half-lives of such moieties -L¹-. Compounds marked with “**” were synthesized for comparison.

### Example 35: Synthesis of Compound 12a

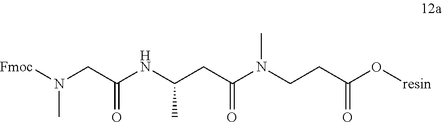

12a was synthesized using solid phase synthesis following the general protocol using Fmoc-N-methyl-beta-alanine, Fmoc-beta-homoalanine-OH and Fmoc-Sar-OH as building blocks.

### Example 36: Synthesis of Compound 12b

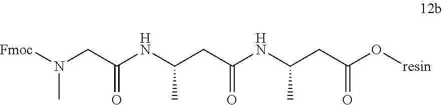

12b was synthesized using solid phase synthesis following the general protocol using Fmoc-beta-homoalanine-OH and Fmoc-Sar-OH as building blocks.

### Example 37: Synthesis of Compound 12c

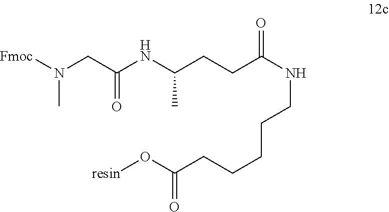

12c was synthesized using solid phase synthesis following the general protocol using Fmoc-Ahx-OH, (S)-Fmoc-4-aminopentanoic acid, and Fmoc-Sar-OH as building blocks.

### Example 38: Synthesis of Compound 12d

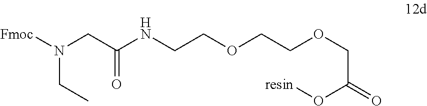

12d was synthesized using solid phase synthesis following the general protocol using Fmoc-8-amino-3,6-dioxaoctanoic acid and Fmoc-N-Ethyl-Gly-OH as building blocks.

### Example 39: Synthesis of Compound 12e

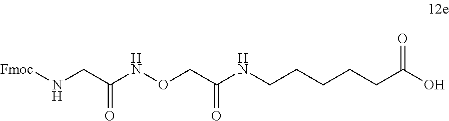

12e was synthesized using solid phase synthesis following the general protocol using Fmoc-Ahx-OH, Fmoc-aminooxyacetic acid and Fmoc-Sar-OH as building blocks and using HFIP for cleavage from the resin. It was then purified by RP-HPLC.

Yield: 2.4 mg

MS: m/z 484.20=[M+H]⁺, (calculated=484.20).

### Example 40: Synthesis of Compounds 13a-d

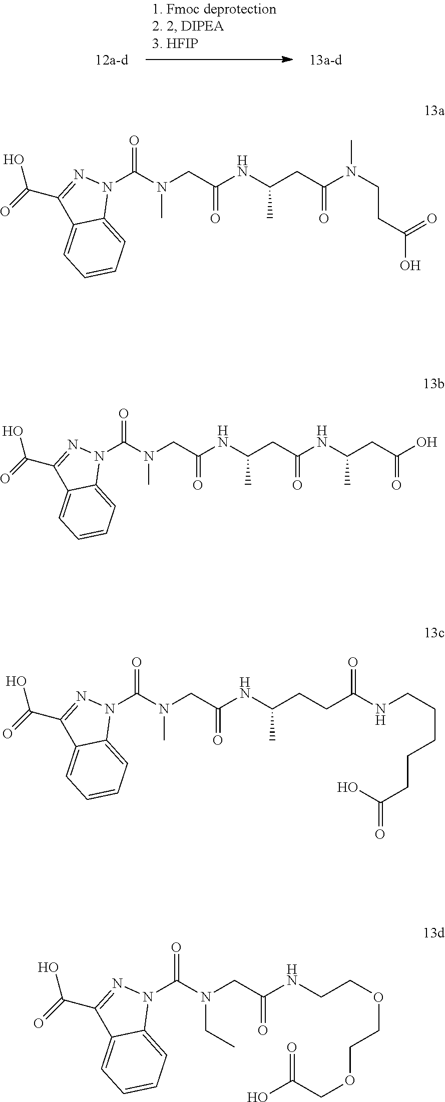 [SMILES: C[C@@H](CC(=O)N(C)CCC(=O)O)NC(=O)CN(C)C(=O)n1nc(C(=O)O)c2ccccc21 ; C[C@@H](CC(=O)O)NC(=O)C[C@H](C)NC(=O)CN(C)C(=O)n1nc(C(=O)O)c2ccccc21 ; C[C@@H](CCC(=O)NCCCCCC(=O)O)NC(=O)CN(C)C(=O)n1nc(C(=O)O)c2ccccc21 ; CCN(CC(=O)NCCOCCOCC(=O)O)C(=O)n1nc(C(=O)O)c2ccccc21]

Conjugates 13a-d were synthesized by Fmoc deprotection of 12a-d using 2:2:96 piperidine/DBU/DMF, following reacting an excess of 2 with the respective amine on resin using excess DIPEA in DMF. The product was cleaved from the resin using HFIP and purified by RP-HPLC.

13a: 12a: 32 mg, 24 μmol, 2: 20 mg, 60 μmol, DIPEA: 17 μL, 96 μmol

Yield: 5.1 mg (48%), MS: m/z 448.15=[M+H]⁺, (calculated=448.18).

13b: 12b: 37 mg, 27 μmol, 2: 22 mg, 68 μmol, DIPEA: 19 μL, 109 μmol

Yield: 5.2 mg (43%), MS: m/z 448.15=[M+H]⁺, (calculated=448.18).

13c: 12c: 32 μmol, 2: 26 mg, 79 μmol, DIPEA: 22 μL, 127 μmol

Yield: 6.5 mg (42%), MS: m/z 490.20=[M+H]⁺, (calculated=490.23).

13d: 12d: 26 μmol, 2: 21 mg, 64 μmol, DIPEA: 18 μL, 102 μmol

Yield: 2.9 mg (26%), MS: m/z 437.14=[M+H]⁺, (calculated=437.16).

### Example 41: Synthesis of Compounds 13e

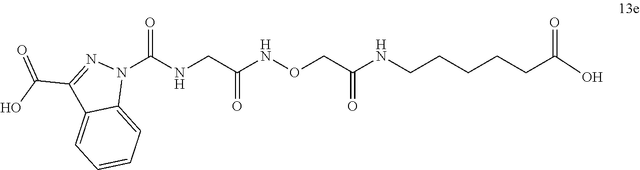 [SMILES: O=C(O)CCCCCNC(=O)CONC(=O)CNC(=O)n1nc(C(=O)O)c2ccccc21]

Compound 12e (2.4 mg, 0.005 mmol) in DMF (0.5 mL) was treated with piperidine (50 μL). After stirring at RT for 1 h, the mixture was diluted with DCM and the volatiles removed in vacuo. The residues were combined with 2 (2.4 mg; 0.007 mmol) in DMF (0.2 mL) and DIPEA (2.6 μL; 0.015 mmol; 3.0 eq.) was added. After stirring at RT for 65 min, TFA (1.1 μL) was added. The volatiles were removed in vacuo and the residues redissolved in 1:3 acetonitrile/H₂O and freeze-dried. The residues were again combined with 2 (2.7 mg; 0.008 mmol) in DMF (0.2 mL) and DIPEA (5.0 μL; 0.029 mmol; 3.0 eq.) was added. After stirring at RT for 2.5 h, TFA (1.5 μL) was added. The volatiles were removed in vacuo and the residues redissolved in 1:3 acetonitrile/H₂O and freeze-dried, and the crude purified by RP-HPLC

Yield: 0.1 mg (3%)

MS: m/z 450.10=[M+H]⁺, (calculated=450.16)

### Example 42: Synthesis of Compound 14a

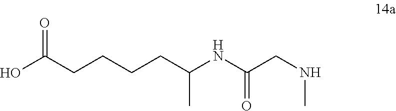

Methyl 6-oxo-heptanoate (2 g, 12.6 mmol) was dissolved in methanol (13 mL) and ammonium acetate (9.75 g, 126 mmol), and sodium cyanoborohydride (1.19 g, 19.0 mmol) was added with stirring. The resulting suspension turned into a solution and stirring was continued overnight. The mixture was diluted with water (70 mL) and ethyl acetate was added (80 mL). The pH of the water phase was adjusted to circa pH 11 with 25 mL 4 M NaOH. The aqueous phase was extracted with ethyl acetate (three times 70 mL). The combined organic phases were dried (MgSO₄), filtered and concentrated in vacuo to give a yellow oil (1.83 g). A portion of the crude oil (200 mg) from the first step was dissolved in DMF (2 mL) and N-Boc-Sar-OH (238 mg, 1.26 mmol), PyBOP (719 mg, 1.38 mmol) and DIPEA (656 μL, 3.77 mmol) were added with stirring. The reaction was stirred at RT for 2 h. The mixture was diluted with 25 mL ethyl acetate and washed with 0.1 N HCl (3 times 15 mL), 0.5 M NaOH (3 times 15 mL) and brine (15 mL). The organic phase was dried (MgSO₄), filtered and concentrated in vacuo. The residue was purified using flash chromatography (heptane/ethyl acetate). The product (235 mg) was dissolved in THF (1 mL), and LiOH (51 mg, 2.13 mmol) was dissolved in water (0.4 mL). The solutions were combined and stirred vigorously at RT. After 5 h the mixture was diluted with 80 mL ethyl acetate, and 60 mL 1 N HCl was added. The pH of the aqueous phase was below 2. The organic phase was collected, and the aqueous phase extracted with ethyl acetate (three times 20 mL). The combined organics were dried (MgSO₄), filtered, and concentrated in vacuo. The residue was dissolved in DCM (1.0 mL) and TFA (0.5 mL) was added with vigorous stirring in an open flask. After 75 min the reaction was concentrated in vacuo and co-evaporated once with 5 mL DCM. The crude was dissolved in 1:2 acetonitrile/H₂O+0.1% TFA (20 mL) and lyophilized.

Yield: 213 mg (47%, TFA salt)

MS: m/z 217.05=[M+H]⁺, (calculated=217.15).

### Example 43: Synthesis of Compound 14b

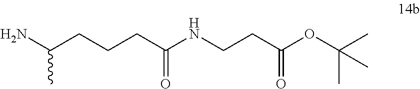 [SMILES: CC(N)CCCC(=O)NCCC(=O)OC(C)(C)C]

Methyl 5-oxohexanoate (2.00 g, 13.9 mmol) was dissolved in THF (60 mL) and LiOH (1.00 g, 41.6 mmol) and water (20 mL) were added. The mixture was stirred at RT for 5 h before dilution with ethyl acetate (300 mL). 1 N aq.HCl (80 mL) was added, and the aqueous phase extracted with ethyl acetate (2 times 100 mL). The combined organics were dried (MgSO₄) and concentrated in vacuo. The resulting colorless oil (1.6 g) was dissolved in DMF (32 mL), and PyBOP (7.68 g, 14.8 mmol) then DIPEA (10.7 mL, 61.5 mmol) were added to the mixture. After stirring for 5 min tert-butyl 3-aminopropanoate hydrochloride (2.69 g, 14.8 mmol) was added and the mixture stirred at RT for 105 min. The mixture was diluted with ethyl acetate (400 mL) and washed with 0.55 M aq. HCl (100 mL), 0.1 M aq. HCl (2 times 100 mL), sat. NaHCO₃ (3 times 100 mL), and brine (100 mL). The organics were dried (MgSO₄) and concentrated in vacuo before being purified by flash chromatography (ethyl acetate/heptane). The purified material was then dissolved in MeOH (14.2 mL) and ammonium acetate (6.60 g, 85.6 mmol) and sodium cyanoborohydride (801 mg; 12.8 mmol) were added. The mixture was stirred overnight at RT. The mixture was diluted with water (70 ml) and ethyl acetate (80 ml). Using 4 M NaOH (15 mL) the pH of the aq. phase was adjusted to ca. pH 2. The aq. phase was extracted with ethyl acetate (3 times 70 mL), the organics combined and TFA (648 μL) added. To the aq. phase was added further 4 M NaOH (5 mL) and again it was extracted with ethyl acetate (3 times 70 mL), these organics were combined and TFA (400 μL) added. The organics were dried (MgSO₄), filtered, and the volatiles removed in vacuo.

Yield: 3.40 g (66%, TFA salt)

MS: m/z 259.12=[M+H]⁺, (calculated=259.20).

### Example 44: Synthesis of Compound 14c

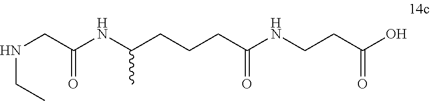 [SMILES: CCNCC(=O)NC(C)CCCC(=O)NCCC(=O)O]

Compound 14b (249 mg, 0.62 mmol) was dissolved in DMF (2.30 mL) and N-Boc-N-ethylglycine (132 mg, 0.65 mmol) and PyBOP (353 mg; 0.68 mmol) were added followed by DIPEA (326 μL, 1.87 mmol) to form a light yellow solution. After stirring at RT for 90 min, the mixture was diluted with ethyl acetate (50 mL) and washed with 0.1 M HCl (3 times 25 mL), sat. aq. NaHCO₃ (25 mL), a 3:5 v/v mixture of brine and sat. aq. NaHCO₃ (2 times 40 mL), and brine (30 mL). The organics were dried (MgSO₄) and the voaltiles removed in vacuo. The intermediate was purified by flash chromatography (methanol/DCM) and then purified by RP-HPLC to give a colourless oil. The oil was dissolved in DCM (0.5 mL) and TFA (0.5 mL) was added. After stirring at RT for 55 min, the volatiles were removed under a stream of nitrogen. The residues were diluted with acetonitrile/H₂O 1:1+0.1% TFA (2 mL)+0.1% TFA and water (4 mL). The mixture was lyophilized to give a colourless oil.

Yield: 136 mg (52%, TFA salt)

MS: m/z 288.19=[M+H]⁺, (calculated=288.19).

### Example 45: Synthesis of Compound 14d

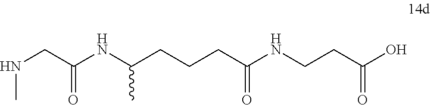 [SMILES: CNCC(=O)NC(C)CCCC(=O)NCCC(=O)O]

Compound 14b (251 mg, 0.63 mmol) was dissolved in DMF (2.30 mL) and N-Boc-Sar-OH (121 mg, 0.64 mmol) and PyBOP (358 mg; 0.69 mmol) were added followed by DIPEA (326 μL, 1.87 mmol) to form a light-yellow solution. After stirring at RT for 90 min, the mixture was diluted with ethyl acetate (50 mL) and washed with 0.1 M HCl (3 times 25 mL), sat. aq. NaHCO₃ (25 mL), a 3:5 v/v mixture of brine and sat. aq. NaHCO₃ (2 times 40 mL), and brine (30 mL). The organics were dried over MgSO₄ and the voaltiles removed in vacuo. The intermediate was purified by flash chromatography (methanol/DCM) to give a colourless oil. The oil was dissolved in DCM (0.5 mL) and the solution treated with TFA (0.5 mL). After stirring at RT for 55 min, the volatiles were removed under a stream of nitrogen. The residues were diluted with 1:1 acetonitrile/H₂O+0.1% TFA (2 mL)+0.1% TFA and water (4 mL). The mixture lyophilized to give a colourless oil.

Yield: 129 mg (51%, TFA salt)

MS: m/z 274.18=[M+H]⁺, (calculated=274.17).

### Example 46: Synthesis of Compound 14e

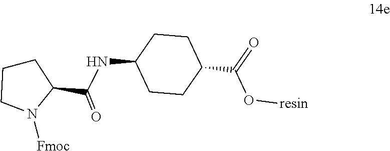

14e was synthesized using solid-phase synthesis following the general protocol using Fmoc-trans-1,4-ACHC-OH and Fmoc-Pro-OH as building blocks.

### Example 47: Synthesis of Compound 14f

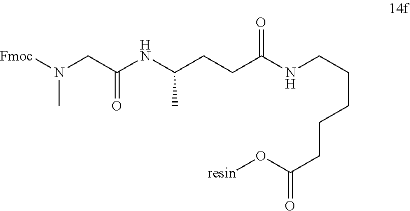

14f was synthesized using solid-phase synthesis following the general protocol using Fmoc-Ahx-OH, (S)-Fmoc-4-aminopentanoic acid, and Fmoc-N-Methyl-Ala-OH as building blocks.

### Example 48: Synthesis of Compound 14g

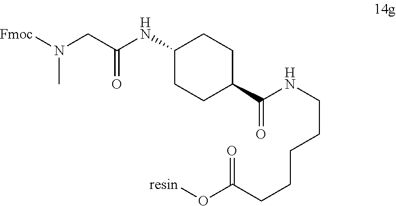

14g was synthesized using solid-phase synthesis following the general protocol using Fmoc-Ahx-OH, Fmoc-trans-1,4-ACHC-OH, and Fmoc-Sar-OH as building blocks.

### Example 49: Synthesis of Compound 14h

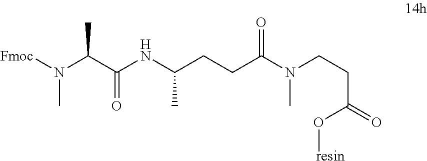

14h was synthesized using solid-phase synthesis following the general protocol using Fmoc-N-Methyl-β-Ala-OH, (S)-Fmoc-4-aminopentanoic acid, and Fmoc-N-Methyl-Ala-OH as building blocks.

### Example 50: Synthesis of Compound 14i

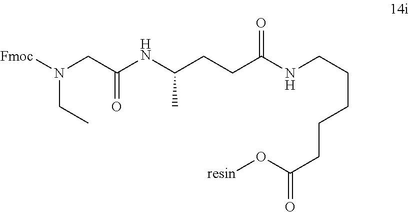

14i was synthesized using solid-phase synthesis following the general protocol using Fmoc-Ahx-OH, (S)-Fmoc-4-aminopentanoic acid, and Fmoc-N-Ethyl-Gly-OH as building blocks.

### Example 51: Synthesis of 15a

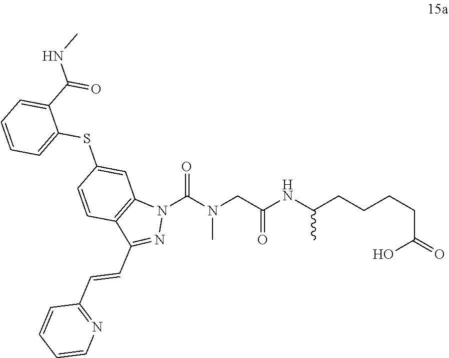 [SMILES: CNC(=O)c1ccccc1Sc1ccc2c(/C=C/c3ccccn3)nn(C(=O)N(C)CC(=O)NC(C)CCCCC(=O)O)c2c1]

14a (213 mg, 0.64 mmol) was dissolved in 500 μL of DMF and DIPEA (247 μL, 1.42 mmol) was added. A suspension of 3 (162 mg, 0.28 mmol, in 2.0 mL DMF) was added. After 2 h TFA (110 μl, 1.44 mmol) was added and the product purified by RP-HPLC.

Yield: 111 mg (54%, TFA salt)

MS: m/z 629.20=[M+H]⁺, (calculated=629.25).

### Example 52: Synthesis of 15b

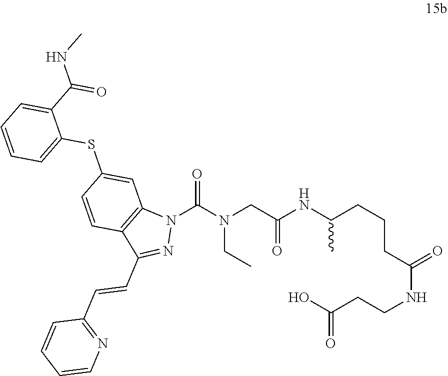 [SMILES: CCN(CC(=O)NC(C)CCCC(=O)NCCC(=O)O)C(=O)n1nc(/C=C/c2ccccn2)c2ccc(Sc3ccccc3C(=O)NC)cc21]

Compound 14c (136 mg, 0.338 mmol) was dissolved in DMF (0.25 mL) and DIPEA (147 μL, 0.845 mmol) was added. To the stirred colourless solution was added 3 (100 mg, 0.169 mmol) in DMF (1.23 mL) and the mixture immediately turned clear yellow. The mixture was stirred at RT for 105 min then TFA (65 μL, 0.845 mmol) was added. The product was purified by RP-HPLC to give a yellow solid.

Yield: 100 mg (72%, TFA salt)

MS: m/z 700.24=[M+H]⁺, (calculated=700.29).

### Example 53: Synthesis of 15c

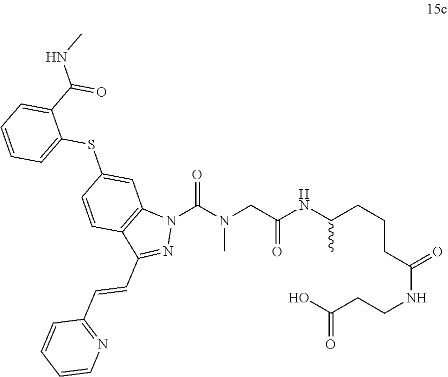 [SMILES: CNC(=O)c1ccccc1Sc1ccc2c(/C=C/c3ccccn3)nn(C(=O)N(C)CC(=O)NC(C)CCCC(=O)NCCC(=O)O)c2c1]

Compound 14d (129 mg, 0.333 mmol) was dissolved in DMF (0.25 mL) and DIPEA (145 μL, 0.83 mmol) was added. To the stirred colourless solution was added 3 (98 mg, 0.169 mmol) in DMF (1.21 mL) and the mixture immediately turned clear yellow. The mixture was stirred at RT for 105 min then TFA (65 μL, 0.845 mmol) was added. The product was purified by RP-HPLC to give a yellow solid.

Yield: 101 mg (75%, TFA salt)

MS: m/z 686.19=[M+H]⁺, (calculated=686.27).

### Example 54: Synthesis of 15d-h

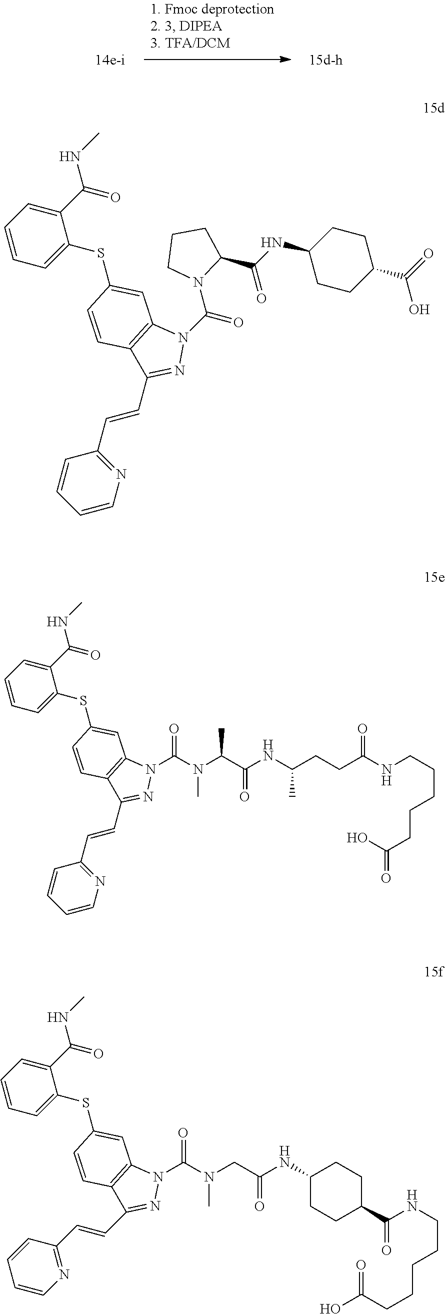 [SMILES: CNC(=O)c1ccccc1Sc1ccc2c(/C=C/c3ccccn3)nn(C(=O)N3CCC[C@H]3C(=O)N[C@H]3CC[C@H](C(=O)O)CC3)c2c1 ; CNC(=O)c1ccccc1Sc1ccc2c(/C=C/c3ccccn3)nn(C(=O)N(C)[C@@H](C)C(=O)N[C@@H](C)CCC(=O)NCCCCCC(=O)O)c2c1 ; CNC(=O)c1ccccc1Sc1ccc2c(/C=C/c3ccccn3)nn(C(=O)N(C)CC(=O)N[C@H]3CC[C@H](C(=O)NCCCCCC(=O)O)CC3)c2c1]

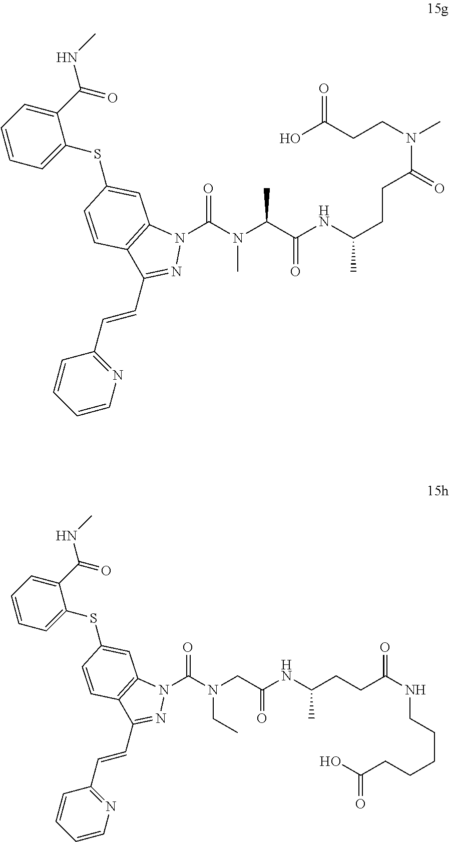 [SMILES: CNC(=O)c1ccccc1Sc1ccc2c(/C=C/c3ccccn3)nn(C(=O)N(C)[C@@H](C)C(=O)N[C@@H](C)CCC(=O)N(C)CCC(=O)O)c2c1 ; CCN(CC(=O)N[C@@H](C)CCC(=O)NCCCCCC(=O)O)C(=O)n1nc(/C=C/c2ccccn2)c2ccc(Sc3ccccc3C(=O)NC)cc21]

The conjugates 15d-h were prepared from their respective resin-loaded Fmoc-protected amines 14e-i, which were treated with 96:2:2 DMF/piperidine/DBU (5 ml) and shaken for 15 min at RT. The filtrate was drained and the procedure repeated twice before washing of the resin with DMF (5 times). The resin was then treated with a suspension of 3 in DMF and DIPEA. The mixture was shaken at RT for between 90 and 200 min before being washed with DMF (5 times) and DCM (5 times). The resin was treated with 1:9 TFA/DCM and shaken at RT for 10 min. The filtrate was collected and this was repeated at least once. The volatiles were removed from the combined filtrates in vacuo to give the acid.

15d: 14e: 493 mg, 0.453 mmol, 3: 266 mg, 0.453 mmol, DMF: 3.5 mL, DIPEA: 485 μL, 2.72 mmol.

Yield: 331 mg (95%, TFA salt). MS: m/z=653.29 [M+H]⁺, (calculated=653.25).

15e: 14f: 195 mg, 0.16 mmol, 3: 119 mg, 0.20 mmol, DMF: 1.5 mL, DIPEA: 173 μL, 0.97 mmol.

Yield: 136 mg (quant., TFA salt). MS: m/z=728.36 [M+H]⁺, (calculated=728.32).

15f: 14g: 312 mg, 0.26 mmol, 3:188 mg, 0.32 mmol, DMF: 2.3 mL, DIPEA: 275 μL, 1.54 mmol.

Yield: 274 mg (quant., TFA salt). MS: m/z=740.34 [M+H]⁺, (calculated=740.32).

15g: 14h: 170 mg, 0.15 mmol, 3: 108 mg, 0.18 mmol, DMF: 1.3 mL DIPEA: 158 μL, 0.88 mmol.

Yield: 124 mg (quant., TFA salt). MS: m/z=700.32 [M+H]⁺, (calculated=700.29).

15h: 14i: 201 mg, 0.17 mmol 3: 123 mg, 0.21 mmol, DMF: 1.5 mL DIPEA: 0.18 mL, 1.00 mmol.

Yield: 155 mg (quant., TFA salt). MS: m/z=728.34 [M+H]⁺, (calculated=728.32).

### Example 55: Synthesis of Compounds 16a-f

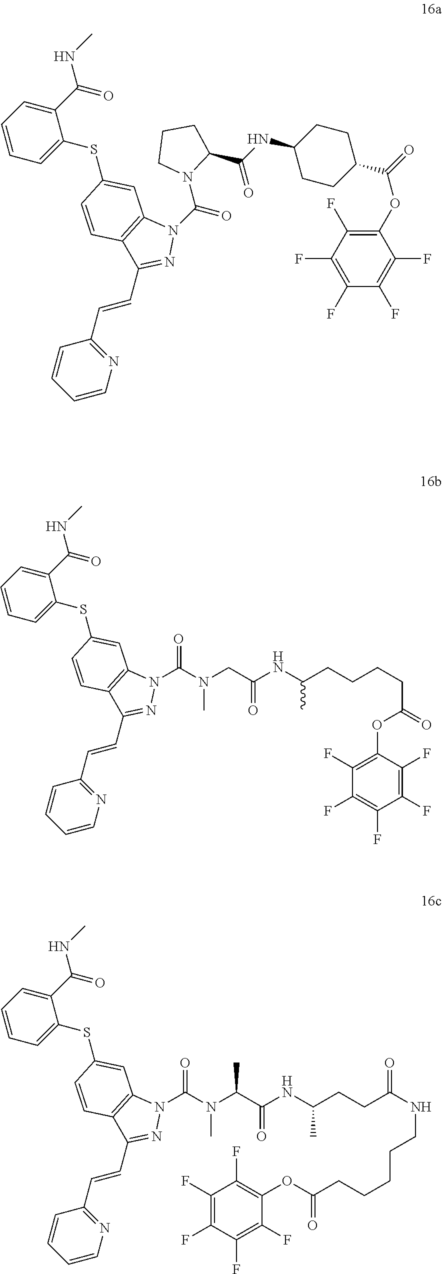 [SMILES: CNC(=O)c1ccccc1Sc1ccc2c(/C=C/c3ccccn3)nn(C(=O)N3CCC[C@H]3C(=O)N[C@H]3CC[C@H](C(=O)Oc4c(F)c(F)c(F)c(F)c4F)CC3)c2c1 ; CNC(=O)c1ccccc1Sc1ccc2c(/C=C/c3ccccn3)nn(C(=O)N(C)CC(=O)NC(C)CCCCC(=O)Oc3c(F)c(F)c(F)c(F)c3F)c2c1 ; CNC(=O)c1ccccc1Sc1ccc2c(/C=C/c3ccccn3)nn(C(=O)N(C)[C@@H](C)C(=O)N[C@@H](C)CCC(=O)NCCCCCC(=O)Oc3c(F)c(F)c(F)c(F)c3F)c2c1]

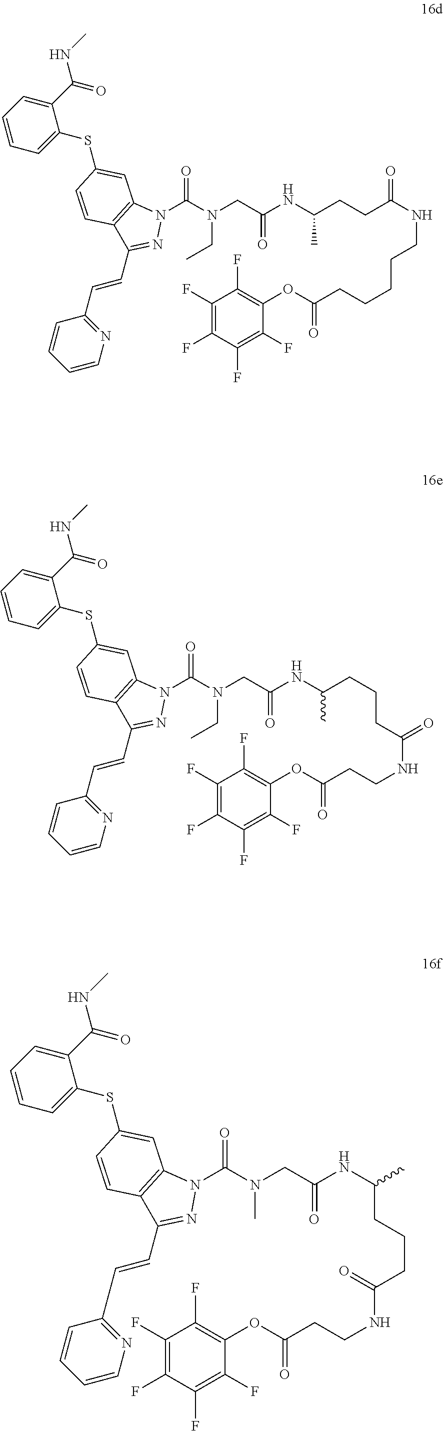

The respective acid selected from 15a-e, h was dissolved in DCM and Bis(pentafluorophenyl) carbonate was added. DIPEA was added and the reaction stirred at RT. Once the reaction was complete it was quenched with TFA and the product purified by flash chromatography (THF/ethyl acetate).

16a: DCM: 4.0 mL, Bis(pentafluorophenyl) carbonate: 213 mg, 0.54 mmol, DIPEA: 377 μL, 2.16 mmol, 15d: 331 mg, 0.43 mmol, TFA: 165 μL, 2.16 mmol.

Yield: 273 mg (68%, TFA salt). MS: m/z 819.34=[M+H]⁺, (calculated=819.23).

16b: DCM: 2.0 mL, Bis(pentafluorophenyl) carbonate: 70 mg, 0.178 mmol, DIPEA: 130 μL, 0.746 mmol, 15a: 111 mg, 0.149 mmol, TFA: 57 μL, 0.746 mmol.

Yield: 122 mg (90%, TFA salt). MS: m/z 795.25=[M+H]⁺, (calculated=795.23).

16c: DCM: 1.50 mL, Bis(pentafluorophenyl) carbonate: 91 mg, 0.23 mmol, DIPEA: 162 μL, 0.93 mmol, 15e: 156 mg, 0.19 mmol, TFA: 71 μL, 0.93 mmol.

Yield: 94 mg (50%, TFA salt). MS: m/z 894.30=[M+H]⁺, (calculated=894.30).

16d: DCM: 1.50 mL, Bis(pentafluorophenyl) carbonate: 91 mg, 0.23 mmol, DIPEA: 161 μL, 0.92 mmol, 15h: 155 mg, 0.18 mmol, TFA: 71 μL, 0.92 mmol.

Yield: 105 mg (56%, TFA salt). MS: m/z 894.31=[M+H]⁺, (calculated=894.30).

16e: DCM: 2.00 mL, Bis(pentafluorophenyl) carbonate: 58 mg, 0.147 mmol, DIPEA: 107 μL, 0.61 mmol, 15b: 100 mg, 0.122 mmol, TFA: 47 μL, 0.61 mmol.

Yield: 77 mg (64%, TFA salt). MS: m/z=866.26 [M+H]⁺, (calculated=866.27).

16f: DCM: 2.00 mL, Bis(pentafluorophenyl) carbonate: 59 mg, 0.151 mmol, DIPEA: 110 μL, 0.63 mmol, 15c: 101 mg, 0.126 mmol, TFA: 48 μL, 0.63 mmol.

Yield: 96 mg (79%, TFA salt). MS: m/z=852.21 [M+H]⁺, (calculated=852.26).

### Example 56: Synthesis of Compound 16g

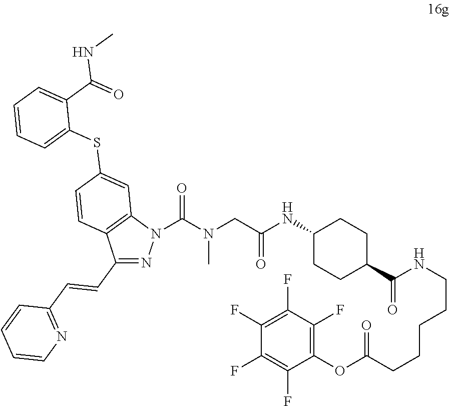 [SMILES: CNC(=O)c1ccccc1Sc1ccc2c(/C=C/c3ccccn3)nn(C(=O)N(C)CC(=O)N[C@H]3CC[C@H](C(=O)NCCCCCC(=O)Oc4c(F)c(F)c(F)c(F)c4F)CC3)c2c1]

To a solution of 15f (274 mg, 0.32 mmol) in DCM (2.5 mL) was added bis(pentafluorophenyl) carbonate (158 mg, 0.40 mmol) followed by DIPEA (280 μL, 1.60 mmol). Further DCM (2.5 mL) and DIPEA (280 μL, 1.60 mmol) were added to the suspension. acetonitrile (1 mL) and DMF (2 mL) were added. The suspension was stirred at RT for 1 d. The mixture was filtered, and the precipitate washed with DCM. The combined filtrates were washed with water, dried (Na₂SO₄), filtered and concentrated in vacuo. The concentrate was diluted with DCM before addition of TFA (245 μL, 3.18 mmol) and the product purified by flash chromatography (THF/ethyl acetate).

Yield: 66 mg (20%, TFA salt)

MS: m/z 906.41=[M+H]⁺, (calculated=906.30).

### Example 57: Synthesis of Compound 16h

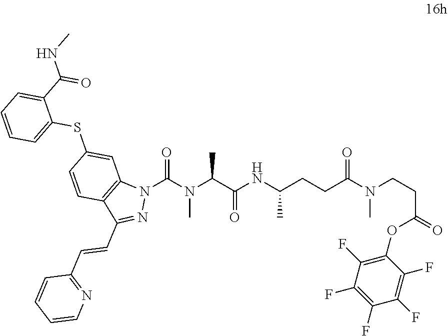 [SMILES: CNC(=O)c1ccccc1Sc1ccc2c(/C=C/c3ccccn3)nn(C(=O)N(C)[C@@H](C)C(=O)N[C@@H](C)CCC(=O)N(C)CCC(=O)Oc3c(F)c(F)c(F)c(F)c3F)c2c1]

To a solution of 15g (124 mg, 0.15 mmol) in DCM (1.5 mL) was added Bis(pentafluorophenyl) carbonate (75 mg, 0.19 mmol) followed by DIPEA (133 μL, 0.76 mmol). After stirring at RT for 3 h further Bis(pentafluorophenyl) carbonate (19 mg, 0.05 mmol) was added, and after a further 1 h DIPEA (65 μL, 0.37 mmol) was added. The mixture was left to stir at RT for another 18 h. The product was purified directly by flash chromatography (THF/ethyl acetate).

Yield: 24 mg (16%, TFA salt)

MS: m/z 866.30=[M+H]⁺, (calculated=866.27).

### Example 58: Synthesis of Compounds 17a-q

Various hydrogels (amine content of 0.564-0.934 mmol/g) were reacted with Axitinib-linker-conjugates according to the following scheme:

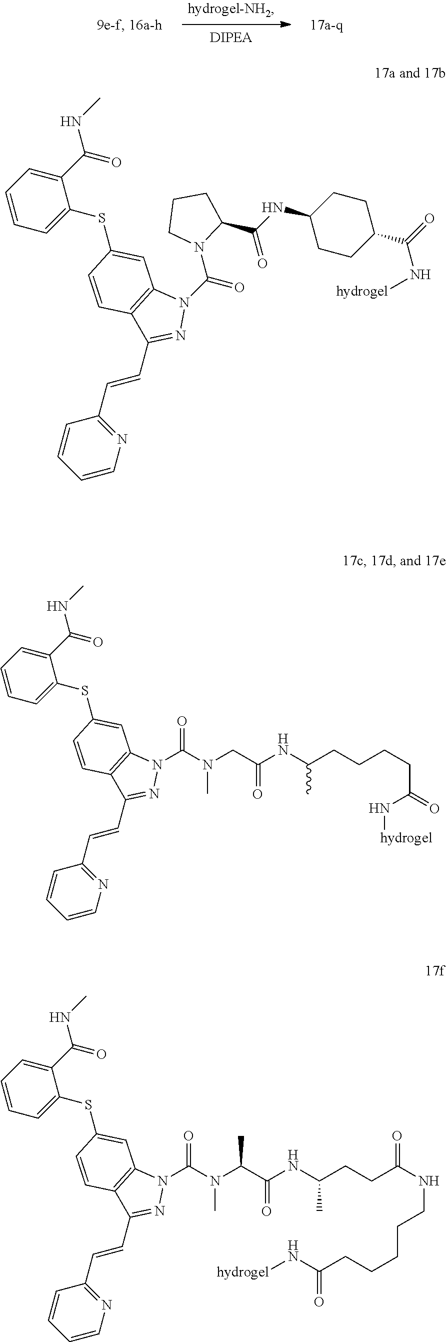

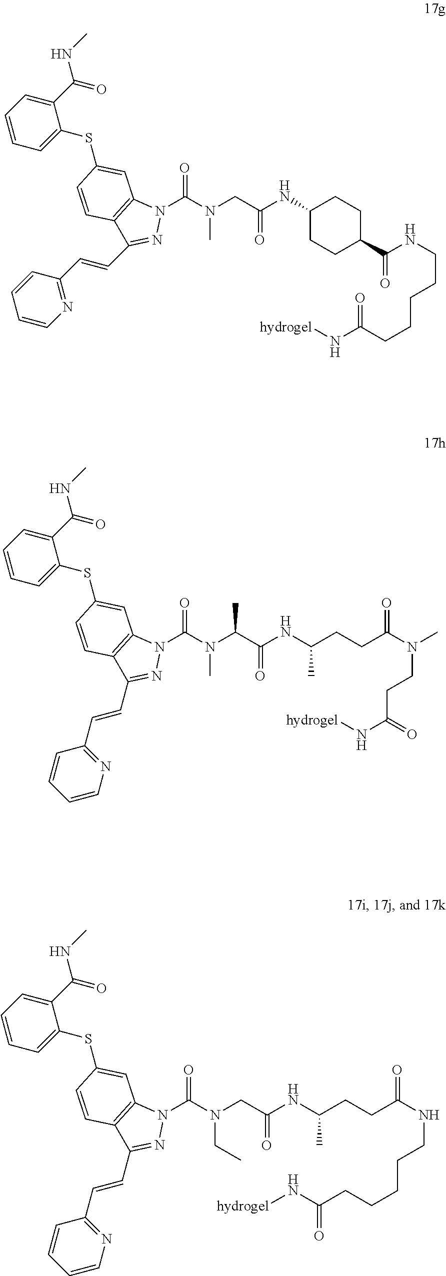

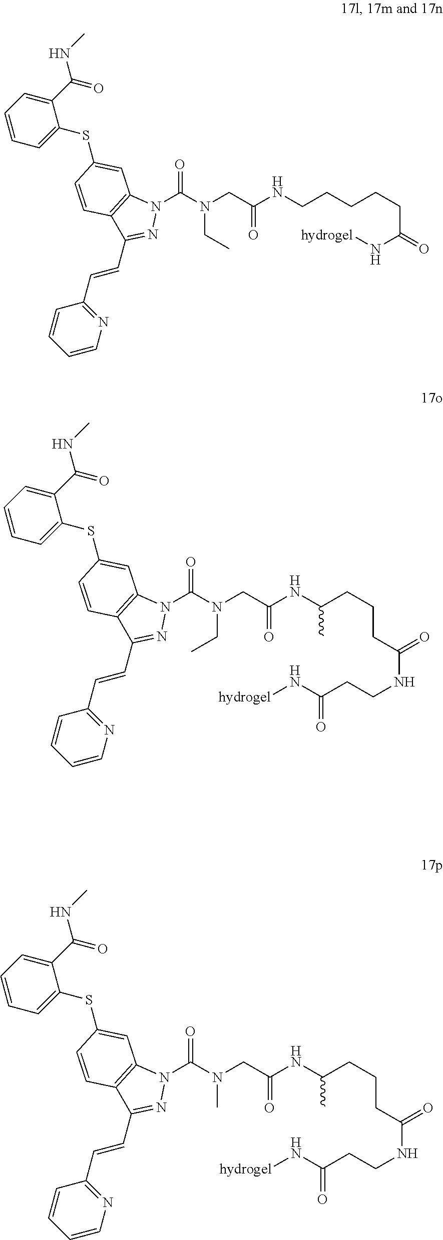

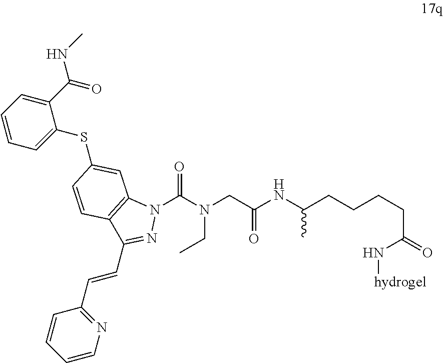

The hydrogel was swollen in 1% DIPEA in DMF in a syringe reactor containing a PE frit. The syringe reactor was 3 times filled, shaken for 1 min and drained. A PFP-ester selected from 9e-f or 16a-h was dissolved in DMF and DIPEA was added. The solution was drawn into the syringe containing the hydrogel. The syringe was shaken for longer than 16 h at RT. The syringe was drained, and the hydrogel was washed several times with DMF, then water, then pH 5.5 20 mM sodium succinate aqueous buffer. A hydrogel suspension in pH 5.5 aqueous buffer was obtained. The proportion of amines from the hydrogel that were conjugated was determined by comparing the determined drug content of the product with the amine content of the starting amine hydrogels.

17a: HG-6: 21 mg, DIPEA: 12.1 μL, 16a: 29 mg

yield: suspension, 94% Axitinib loading, 17.93 mg/mL axitinib in hydrogel suspension.

17b: HG-12: 20 mg, DIPEA: 10.8 μL, 16a: 27 mg

yield: suspension, 69% Axitinib loading, 11.69 mg/mL axitinib in hydrogel suspension.

17c: HG-11: 19 mg, DIPEA: 9.3 μL, 16b: 17 mg

yield: suspension, 100% Axitinib loading, 16.24 mg/mL axitinib in hydrogel suspension.

17d: HG-8: 20 mg, DIPEA: 11.7 μL, 16b: 22 mg

yield: suspension, 95% Axitinib loading, 16.10 mg/mL axitinib in hydrogel suspension.

17e: HG-13: 16 mg, DIPEA: 9.5 μL, 16b: 18 mg

yield: suspension, 97% Axitinib loading, 18.63 mg/mL axitinib in hydrogel suspension.

17f: HG-11: 19 mg, DIPEA: 9.5 μL, 16c: 20 mg

yield: suspension, 100% Axitinib loading, 16.43 mg/mL axitinib in hydrogel suspension.

17g: HG-11: 20 mg, DIPEA: 10.0 μL, 16g: 21 mg

yield: suspension, 95% Axitnib loading, 14.03 mg/mL axitinib in hydrogel suspension.

17h: HG-11: 20 mg, DIPEA: 9.9 μL, 16h: 24 mg

yield: suspension, 84% Axintib loading, 11.17 mg/mL axitinib in hydrogel suspension.

17i: HG-11: 20 mg, DIPEA: 9.9 μL, 16d: 20 mg

yield: suspension, 94% Axitinib loading, 14.66 mg/mL axitinib in hydrogel suspension.

17j: HG-8: 20 mg, DIPEA: 11.8 μL, 16d: 25 mg

yield: suspension, 96% Axitinib loading, 15.72 mg/mL axitinib in hydrogel suspension.

17k: HG-13: 15 mg, DIPEA: 9.1 μL, 16d: 19 mg

yield: suspension, 100% Axitinib loading, 19.46 mg/mL axitinib in hydrogel suspension.

17l: HG-4: 15 mg, DIPEA: 8.5 μL, 9e: 15 mg

yield: suspension, 98% Axitinib loading, 18.27 mg/mL axitinib in hydrogel suspension.

17m: HG-11: 21 mg, DIPEA: 10.2 μL, 9e: 19 mg

yield: suspension, 100% Axitinib loading, 15.60 mg/mL axitinib in hydrogel suspension.

17n: HG-14: 20 mg, DIPEA: 16.2 μL, 9e: 32 mg

yield: suspension, 81% Axitinib loading, 21.15 mg/mL axitinib in hydrogel suspension.

17o: HG-13: 16 mg, DIPEA: 9.4 μL, 16e: 19 mg

yield: suspension, 99% Axitinib loading, 19.28 mg/mL axitinib in hydrogel suspension.

17p: HG-13: 16 mg, DIPEA: 9.7 μL, 16f: 19 mg

yield: suspension, 100% Axitinib loading, 20.66 mg/mL axitinib in hydrogel suspension.

17q: HG-15: 20 mg, DIPEA: 10.6 μL, 9f: 21 mg

yield: suspension, 93% Axitinib loading, 15.75 mg/mL axitinib in hydrogel suspension.

### Example 59: In Vitro Release Kinetics

The cleavage rate of the reversible bond from conjugates 9a-d, 13a-e and 17b-q was monitored at 37° C. in aqueous buffer (condition A: pH 7.4 60 mM sodium phosphate, 1% acetonitrile, B: pH 7.4 48 mM sodium phosphate, 20% acetonitrile, 0.1% Pluronic F68, C: pH 7.4 48 mM sodium phosphate with 16 mM L-Methionine 2.4 mM EDTA, 0.1% pluronic and 20% acetonitrile, D: pH 7.0 48 mM sodium phosphate with 16 mM L-Methionine 2.4 mM EDTA, 0.1% pluronic and 20% acetonitrile, E: pH 7.4 60 mM sodium phosphate, F: pH 7.4 48 mM sodium phosphate, 20% acetonitrile). For soluble examples disappearance of the conjugate was determined by LCMS (UV detection) and fitted with curve fitting software to obtain the half-life of the release. For insoluble examples (hydrogels) the increase in released axitinib in the supernatant was determined by LCMS (UV detection) and used as input for the curve fitting software to obtain the half-life of the release. Release rates at pH 7.4 for conjugates only incubated at pH 7.0 are estimated to increase by a factor of 2 to 3.

| Compound | t_{1/2} | pH | Buffer | Released product |
|---|---|---|---|---|
| 9a* | 4.0 d | 7.4 | F | axitinib |
| 9b* | 11.5 d | 7.4 | F | axitinib |
| 9c* | 47 d | 7.4 | F | axitinib |
| 9d* | 4.6 d | 7.4 | F | axitinib |
| 13a* | 4.2 d | 7.4 | F | 1H-indazole-3-carboxylic acid |
| 13b* | 3.6 d | 7.4 | F | 1H-indazole-3-carboxylic acid |
| 13c* | 6.9 d | 7.4 | F | 1H-indazole-3-carboxylic acid |
| 13d* | 3.2 d | 7.4 | F | 1H-indazole-3-carboxylic acid |
| 13e* | 4.5 d | 7.4 | F | 1H-indazole-3-carboxylic acid |
| 17b | 107 d | 7.0 | D | axitinib |
| 17b | 181 d | 7.4 | C | axitinib |
| 17c | 28 d | 7.0 | D | axitinib |
| 17c | 12 d | 7.4 | C | axitinib |
| 17d | 26 d | 7.0 | D | axitinib |
| 17e | 30 d | 7.0 | D | axitinib |
| 17f | 49 d | 7.0 | D | axitinib |
| 17f | 20 d | 7.4 | C | axitinib |
| 17g | 17 d | 7.0 | D | axitinib |
| 17g | 7 d | 7.4 | C | axitinib |
| 17h | 51 d | 7.0 | D | axitinib |
| 17h | 21 d | 7.4 | C | axitinib |
| 17i | 27 d | 7.0 | D | axitinib |
| 17i | 12 d | 7.4 | C | axitinib |
| 17j | 24 d | 7.0 | D | axitinib |
| 17k | 30 d | 7.0 | D | axitinib |
| 171 | 17 d | 7.0 | D | axitinib |
| 17m | 16 d | 7.0 | D | axitinib |
| 17m | 7 d | 7.4 | C | axitinib |
| 17n | 8 d | 7.4 | B | axitinib |
| 17o | 43 d | 7.0 | D | axitinib |
| 17p | 15 d | 7.0 | D | axitinib |
| 17q | 38 d | 7.4 | C | axitinib |

The compounds marked with “*” are not in accordance with the present invention as they were for efficiency reasons not linked to a moiety Z. Nevertheless, they show the release half-lives of such moieties -L¹-.

### Example 60: Synthesis of Compounds 17r-t

The hydrogel HG-17 was reacted with Axitinib-linker-conjugates according to the following scheme:

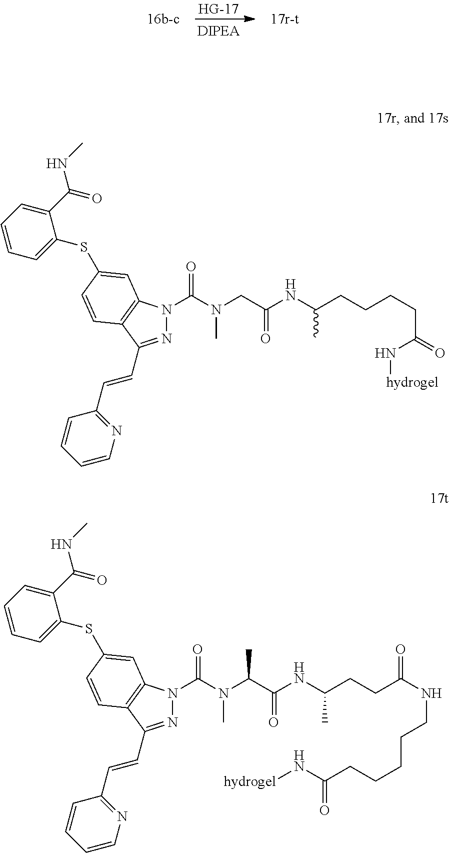

The hydrogel was swollen in 1% DIPEA in DMF in a syringe reactor containing a PE frit. The syringe reactor was 3 times filled, shaken for 1 min and drained. A PFP-ester selected from 16b or 16c was dissolved in DMF and DIPEA was added. The solution was drawn into the syringe containing the hydrogel. The syringe was shaken for longer than 16 h at RT. The syringe was drained, and the hydrogel was washed several times with DMF, then water, then pH 5.5 buffer (20 mM sodium succinate, 77 g/l trehalose dihydrate, 0.2% Pluronic F-68). A hydrogel suspension in pH 5.5 aqueous buffer was obtained. The proportion of amines from the hydrogel that were conjugated was determined by comparing the determined drug content of the product with the amine content of the starting amine hydrogels.

17r: HG-17: 24 mg, DIPEA: 17.8 μL, 16b: 15 mg

yield: suspension, 73% Axitinib loading, 17.63 mg/mL axitinib in hydrogel suspension.

17s: HG-17: 24 mg, DIPEA: 18.1 μL, 16b: 20 mg

yield: suspension, 94% Axitinib loading, 22.40 mg/mL axitinib in hydrogel suspension.

17t: HG-17: 24 mg, DIPEA: 18.2 μL, 16c: 17 mg

yield: suspension, 62% Axitinib loading, 14.87 mg/mL axitinib in hydrogel suspension.

### Example 61: In Vitro Release Kinetics

The cleavage rate of the reversible bond from conjugates 17r-t was monitored at 37° C. in pH 7.0 48 mM sodium phosphate buffer with 16 mM L-Methionine 2.4 mM EDTA, 0.1% pluronic and 20% acetonitrile. The increase in released axitinib in the supernatant was determined by LCMS (UV detection) and used as input for the curve fitting software to obtain the half-life of the release. The release rates at pH 7.4 for these conjugates are estimated to be faster by a factor of 2 to 3.

| Compound | t_{1/2} | Released product |
|---|---|---|
| 17r | 20 d | axitinib |
| 17s | 34 d | axitinib |
| 17t | 31 d | axitinib |

### Example 62

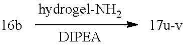

For 17u, a PEG based amino hydrogel is synthesized as described in example 3 of WO2011/012715A1 using a backbone synthesized using Boc-L-Lys(Boc)-OH as described in example 1 of WO2011/012715A1, and a 2 kDa PEG based crosslinker that is synthesized using adipic acid as described in example 2 of WO2011/012715A1. The hydrogel is then modified with lysine using Fmoc-L-Lys(Fmoc)-OH as described in example 5 of WO2011/042450A1 to give a hydrogel with an amine content of 0.700 mmol/g. The hydrogel is swollen in 1% DIPEA in DMF in a syringe reactor fitted with a frit and washed three times with a 1% DIPEA/DMF solution. 16b (1.8 eq. per hydrogel amine) is dissolved in DMF and DIPEA (5.0 eq.) is added. The solution is drawn into the hydrogel-containing reactor and shaken for 16 h at rt. The syringe is drained, the hydrogel washed several times with DMF, washed several times with water, then washed several times with pH 5.5 20 mM sodium succinate aqueous buffer. A hydrogel suspension in pH 5.5 aqueous buffer where the Axitinib loading is greater than 95% is obtained.

The hydrogel 17v is prepared as described for 17u, but Boc-D-Lys(Boc)-OH is used for the backbone synthesis instead of Boc-L-Lys(Boc)-OH, and Fmoc-D-Lys(Fmoc)-OH instead of Fmoc-L-Lys(Fmoc)-OH is used for the lysine modification.

### Example 63: Synthesis of Compound 18

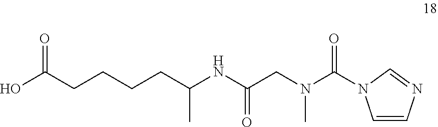 [SMILES: CC(CCCCC(=O)O)NC(=O)CN(C)C(=O)n1ccnc1]

14a (20 mg, 61 μmol) was dissolved in THF (0.5 mL). DIPEA (26 μL, 151 μmol) was added and a precipitate formed. The precipitate was finely distributed using sonication and stirring to form a milky suspension. 1,1′-carbonyldiimidazole (9.8 mg, 61 μmol) was dissolved in 0.15 ml THF and added to the suspension with stirring. After 40 min the reaction was quenched with acetic acid (30 μl) and diluted with water to 1 ml total volume. The product was purified by RP-HPLC to give 18.

Yield: 0.8 mg (4%)

MS: m/z 311.08=[M+H]⁺, (calculated=311.17).

### Example 64: Synthesis of Compound 19

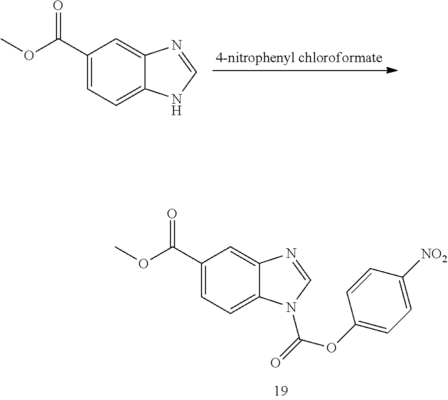 [SMILES: COC(=O)c1ccc2[nH]cnc2c1 ; COC(=O)c1ccc2c(c1)ncn2C(=O)Oc1ccc([N+](=O)[O-])cc1]

A solution of methyl 1H-benzo[d]imidazole-5-carboxylate (25 mg, 117.6 μmol) in THF (500 μl) was cooled to 0° C. and 1-methylimidazole (19.2 μl, 241 μmol) was added. A solution of 4-nitrophenyl chloroformate (24.9 mg, 123.5 μmol) in THF (250 μl) was added dropwise. DMF (500 μl) was added to the reaction mixture to enhance the solubility of a formed precipitate. The mixture was allowed to warm up to ambient temperature in the cooling bath slowly. After 1.5 h additional 1-methylimidazole (19.2 μl, 241 μmol) was added and the reaction mixture was stirred at ambient temperature for approx. 17 h. The reaction mixture is cooled to 0° C. and a solution of 4-nitrophenyl chloroformate (12.5 mg, 62 μmol) in THF (200 μl) was added dropwise. The reaction mixture was stirred at 0° C. for 75 min, then additional 4-nitrophenyl chloroformate (12.5 mg, 62 μmol) in THF (200 μl) was added. The reaction mixture was stirred for 53 min at 0° C., diluted with ethylacetate (ca. 15 ml), washed with 0.1 M HCl (3×5 ml) and brine (ca. 10 ml). The organic layer was dried with Na₂SO₄, filtrated and concentrated under reduced pressure. 19 was purified using flash chromatography.

Yield: 35 mg (87.2%, mixture of benzimidazole regioisomers)

MS: m/z 342.06=[M+H]⁺, (calculated=342.07).

### Example 65: Synthesis of Compound 20

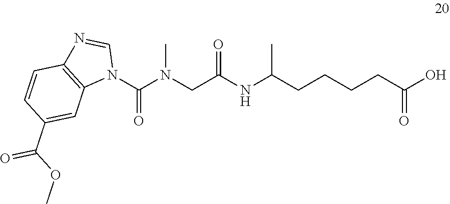 [SMILES: COC(=O)c1ccc2ncn(C(=O)N(C)CC(=O)NC(C)CCCCC(=O)O)c2c1]

To a solution of compound 19 (11.5 mg, 30.3 μmol) in DMF (314 μl) were added DIPEA (21.2 μl, 121.3 μmol) and compound 14a (11.2 mg, 33.4 μmol) successively and the reaction mixture was stirred at ambient temperature for 3 h. The reaction was quenched by addition of TFA (9.3 μl, 121.3 μmol) and the reaction purified by RP-HPLC.

Yield: 2.2 mg (17.3%, mixture of benzimidazole regioisomers)

MS: m/z 419.18=[M+H]⁺, (calculated=419.19).

### Example 66: Synthesis of Compound 21

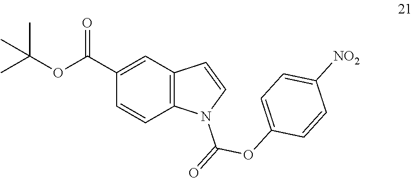 [SMILES: CC(C)(C)OC(=O)c1ccc2c(ccn2C(=O)Oc2ccc([N+](=O)[O-])cc2)c1]

A suspension of NaH (60% in mineral oil, 8.3 mg, 207.5 μmol) in THF (250 μl) was cooled to 0° C. and a solution of tert-butyl 1H-indole-5-carboxylate (15 mg, 69 μmol) in THF (400 μl) was added dropwise. After complete addition the mixture was stirred for 1 h at 0° C. This mixture was added to a cooled solution of 4-nitrophenyl chloroformate (34.8 mg, 172.6 μmol) in THF (500 μl). Additional THF (250 μl) was used to flush the flask and was added to the 4-nitrophenyl chloroformate solution. After 1.5 h the cooling bath was removed, and the mixture was stirred at ambient temperature. After 30 min 4-nitrophenyl chloroformate (69.6 mg, 346.8 μmol) was added in one portion. After 3 h the reaction mixture was diluted with ethyl acetate (15 ml), washed with HCl (0.1 M, 3×5 ml), brine (5 ml), dried with Na₂SO₄, filtrated, and concentrated under reduced pressure. 21 was purified using flash chromatography.

Yield: 16 mg (60.6%)

MS: m/z 327.04=[M -tBu+2H]⁺, (calculated=327.06).

### Example 67: Synthesis of Compound 22

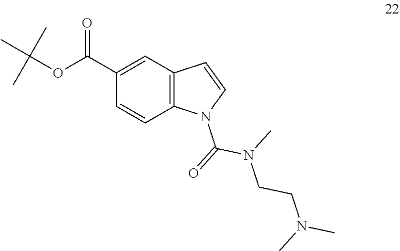 [SMILES: CN(C)CCN(C)C(=O)n1ccc2cc(C(=O)OC(C)(C)C)ccc21]

To a solution of compound 21 (8 mg, 18.8 μmol) in DMF (250 μl) were added DIPEA (13.1 μl, 75.3 μmol) and N,N,N′-trimethylethylenediamine (2.7 μl, 20.7 μmol) successively. The mixture was stirred at ambient temperature for 1 h 45 min before being quenched by addition of TFA (5.6 μl, 75.3 μmol). 22 was purified using RP-HPLC.

Yield: 4.6 mg (70.7%, TFA salt)

MS: m/z 346.18=[M+H]⁺, (calculated=346.21).

### Example 68: Synthesis of Compound 23

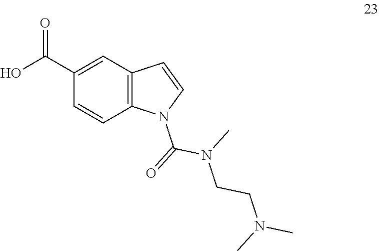 [SMILES: CN(C)CCN(C)C(=O)n1ccc2cc(C(=O)O)ccc21]

To a solution of compound 22 (1.15 mg, 3.3 μmol) in DCM (250 μl) was added TFA (250 μl) and the mixture was stirred at ambient temperature for 25 min. The reaction was diluted with toluene and concentrated under reduced pressure. The obtained material was used directly in the in vitro release kinetics.

MS: m/z 290.15=[M+H]⁺, (calculated=290.15).

### Example 69: In Vitro Release Kinetics

The cleavage rate of the reversible bond from conjugates 18, 20, 23 was monitored at pH 7.4 and 37° C. in aqueous buffer (pH 7.4 48 mM sodium phosphate, 20% acetonitrile). Disappearance of the conjugate was determined by LCMS (UV detection) and fitted with curve fitting software to obtain the half-life of the release.

| Compound | t_{1/2} | Released product |
|---|---|---|
| 18 | 1.7 d | imidazole |
| 20 | 1.8 d | methyl 1H-benzo[d]imidazole-5-carboxylate |
| 23 | 31 d | indole-5-carboxylate |

### Abbreviations

ACHC aminocyclohexane carboxylic acid
Ahx 6-aminohexanoic acid
aq. aqueous
Asp aspartate
Bn benzyl
Boc tert-butyloxycarbonyl
COMU (1-Cyano-2-ethoxy-2-oxoethylidenaminooxy)dimethylamino-morpholino-carbenium hexafluorophosphate
DBU 1,8-diazabicyclo (5.4.0)undec-7-ene
DCM dichloromethane
DIC N,N′-diisopropylcarbodiimide,
DIPEA diisopropylethylamine
DMAP dimethylaminopyridine
DMF dimethylformamide
eq. equivalent
EDC 1-ethyl-3-(3-dimethylaminopropyl)carbodiimide
Fmoc fluorenylmethyloxycarbonyl
HATU O-(7-Azabenzotriazol-1-yl)-N,N,N′,N′-tetramethyluronium-hexafluorphosphat
HFIP 1,1,1,3,3,3-hexafluoroisopropanol
HOBt 1-hydroxybenzotriazole
HPLC high performance liquid chromatography
LC liquid chromatography
LCMS liquid chromatography mass spectrometry
LPLC low pressure liquid chromatography
MeOH methanol
MS mass spectrometry
PEG polyethylene glycol
PFP pentafluorophenyl
PNP para-nitrophenyl
PyBOP benzotriazol-1-yl-oxytripyrrolidinophosphonium hexafluorophosphate
RP reversed phase
RT room temperature
Sar sarcosine
sat. saturated
tBu and t-Bu tert-butyl
TES triethylsilane
TEA triethylamine
TFA trifluoroacetic acid
THF tetrahydrofurane
Thr threonine
Tmob 2,4,6-trimethoxybenzyl
Trt trityl
UPLC ultra performance liquid chromatography
UPLC-MS ultra performance liquid chromatography coupled to mass spectrometry

1. A conjugate or a pharmaceutically acceptable salt thereof comprising at least one moiety -D conjugated via at least one moiety -L¹-L²- to at least one moiety Z, wherein a moiety -L¹- is conjugated to a π-electron-pair-donating heteroaromatic N of a moiety -D and wherein the linkage between -D and -L¹- is reversible and wherein a moiety -L²- is conjugated to Z, wherein
each -D is independently a π-electron-pair-donating heteroaromatic N-comprising moiety of a drug D-H;
each -L²- is independently a single bond or a spacer moiety;
each Z is independently a polymeric moiety or a C₈₋₂₄ alkyl;
each -L¹- is independently a linker moiety of formula (I): 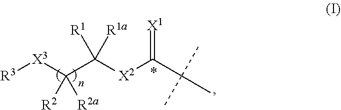
wherein
the dashed line indicates the attachment to the π-electron-pair-donating heteroaromatic N of -D;
n is an integer selected from the group consisting of 0, 1, 2, 3 and 4;
═X¹ is selected from the group consisting of ═O, ═S and ═N(R⁴);
—X²— is selected from the group consisting of —O—, —S—, —N(R⁵)— and —C(R⁶)(R^{6a})—;
—X³— is selected from the group consisting of 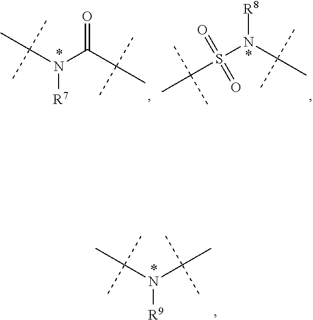
C(R¹⁰)(R^{10a})—, —C(R¹¹)(R^{11a})—C(R¹²)(R^{12a})—, —O— and —C(O)—;
—R¹, —R^{1a}, —R⁶, —R^{6a}, —R¹⁰, —R^{10a}, —R¹¹, —R^{11a}, —R¹², —R^{12a} and each of —R² and —R^{2a} are independently selected from the group consisting of —H, —C(O)OH, halogen, —CN, —OH, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R¹³, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a})—, —S—, —N(R¹⁴), —OC(OR¹⁴)(R^{14a})—, —N(R¹⁴)C(O)N(R^{14a})— and —OC(O)N(R¹⁴)—;
—R³, —R⁴, —R⁵, —R⁷, —R⁸ and —R⁹ are independently selected from the group consisting of —H, -T, —CN, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R¹³, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a})—, —S—, —N(R¹⁴)—, —OC(OR¹⁴)(R^{14a})—, —N(R¹⁴)C(O)N(R^{14a})— and —OC(O)N(R¹⁴)—;
each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl; wherein each T is independently optionally substituted with one or more —R¹³, which are the same or different;
wherein —R¹³ is selected from the group consisting of —H, —NO₂, —OCH₃, —CN, —N(R¹⁴)(R^{14a}), —OH, —C(O)OH and C₁₋₆ alkyl; wherein C₁₋₆ alkyl is optionally substituted with one or more halogen, which are the same or different;
wherein —R¹⁴ and —R^{14a} are independently selected from the group consisting of —H and C₁₋₆ alkyl; wherein C₁₋₆ alkyl is optionally substituted with one or more halogen, which are the same or different;
optionally, one or more of the pairs —R¹/—R^{1a}, —R²/—R^{2a}, two adjacent —R², —R⁶/—R^{6a}, —R¹⁰/—R^{10a}, —R¹¹/—R^{11a}, —R¹²/—R^{12a} and —R³/—R⁹ are joined together with the atom to which they are attached to form a C₃₋₁₀ cycloalkyl, 3- to 10-membered heterocyclyl or an 8- to 11-membered heterobicyclyl;
optionally, one or more of the pairs —R¹/—R², —R¹/—R⁵, —R¹/—R⁶, —R¹/—R⁹, —R¹/—R¹⁰, —R²/—R⁵, —R³/—R^{6a}, —R⁴/—R⁵, —R⁴/—R⁶, —R⁵/—R¹⁰, —R⁶/—R¹⁰ and —R¹¹/—R¹² are joined together with the atoms to which they are attached to form a ring -A-;
wherein -A- is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl;
optionally, —R¹ and an adjacent —R² form a carbon-carbon double bond provided that n is selected from the group consisting of 1, 2, 3 and 4;
optionally, two adjacent —R² form a carbon-carbon double bond provided that n is selected from the group consisting of 2, 3 and 4;
provided that if —X²— is —N(R⁵)—, —X³— is selected from the group consisting of 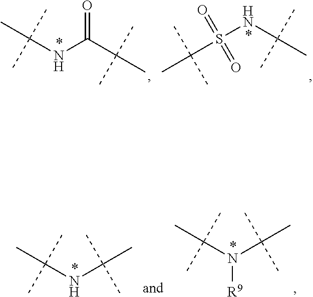
and the distance between the nitrogen atom marked with an asterisk and the carbon atom marked with an asterisk in formula (I) is 5, 6 or 7 atoms and if present the carbon-carbon double bond formed between —R¹ and —R² or two adjacent —R² is in a cis configuration; and
each -L¹- is substituted with -L²- and optionally further substituted.

2. The conjugate or pharmaceutically acceptable salt thereof of claim 1, wherein -D is selected from the group consisting of small molecule, medium size, peptide and protein drug moieties.

3. The conjugate or pharmaceutically acceptable salt thereof of claim 1 or 2, wherein -D is a small molecule drug moiety.

4. The conjugate or pharmaceutically acceptable salt thereof of any one of claims 1 to 3, wherein Z is a polymeric moiety.

5. The conjugate or pharmaceutically acceptable salt thereof of any one of claims 1 to 4, wherein Z is a water-insoluble polymeric moiety.

6. The conjugate or pharmaceutically acceptable salt thereof of any one of claims 1 to 5, wherein Z is a water-insoluble polymeric moiety comprising a polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof.

7. The conjugate or pharmaceutically acceptable salt thereof of any one of claims 1 to 6, wherein Z is a hydrogel.

8. The conjugate or pharmaceutically acceptable salt thereof of any one of claims 1 to 7, wherein Z is a PEG-based or hyaluronic-acid based hydrogel.

9. The conjugate or pharmaceutically acceptable salt thereof of any one of claims 1 to 8, wherein Z is a PEG-based hydrogel.

10. The conjugate or pharmaceutically acceptable salt thereof of any one of claims 1 to 8, wherein Z is a hyaluronic-acid based hydrogel.

11. The conjugate or pharmaceutically acceptable salt thereof of any one of claims 1 to 4, wherein Z is a water-soluble polymeric moiety.

12. The conjugate or pharmaceutically acceptable salt thereof of any one of claims 1 to 11, wherein ═X¹ is ═O and —X²— is —O—.

13. The conjugate or pharmaceutically acceptable salt thereof of any one of claims 1 to 11, wherein ═X¹ is ═O and —X²— is —N(R⁵)—.

14. The conjugate or pharmaceutically acceptable salt thereof of any one of claims 1 to 13, wherein -L²- is a spacer moiety.

15. The conjugate or pharmaceutically acceptable salt thereof of any one of claims 1 to 14, wherein -L²- has a molecular weight in the range of from 14 g/mol to 750 g/mol.

16. The conjugate or pharmaceutically acceptable salt thereof of any one of claims 1 to 15, wherein -L²- is a spacer moiety selected from the group consisting of -T′-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T′-, C₁₋₅₀ alkyl, C₂₋₅₀ alkenyl and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T′-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})— and —OC(O)N(R^{y3})—;
wherein —R^{y1} and —R^{y1a} are independently selected from the group consisting of —H, -T′, C₁₋₅₀ alkyl, C₂₋₅₀ alkenyl and C₂₋₅₀ alkynyl; wherein -T′, C₁₋₅₀ alkyl, C₂₋₅₀ alkenyl, and C₂₋₅₀ alkynyl are optionally substituted with one or more —R^{y2}, which are the same or different, and wherein C₁₋₅₀ alkyl, C₂₋₅₀ alkenyl and C₂₋₅₀ alkynyl are optionally interrupted by one or more groups selected from the group consisting of -T′-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;
each T′ is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl and 8- to 30-membered heteropolycyclyl; wherein each T′ is independently optionally substituted with one or more —R^{y2}, which are the same or different;
each —R^{y2} is independently selected from the group consisting of halogen, —CN, oxo (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and
each —R^{y3}, —R^{y3a}, —R^{y4}, —R^{y4a}, —R^{y5}, —R^{y5a} and —R^{y5b} is independently selected from the group consisting of —H and C₁₋₆ alkyl; wherein C₁₋₆ alkyl is optionally substituted with one or more halogen, which are the same or different.

17. The conjugate or pharmaceutically acceptable salt thereof of any one of claims 1 to 16, wherein one hydrogen given by —R³ is replaced by -L²-.

18. The conjugate or pharmaceutically acceptable salt thereof of any one of claims 1 to 17, wherein the linkage between Z and -L²- is stable.

19. The conjugate or pharmaceutically acceptable salt thereof of any one of claims 1 to 11 and 13 to 18, wherein -L¹- is of formula (Ix): 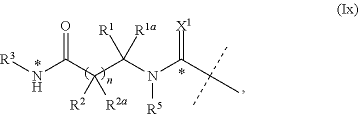
wherein the dashed line indicates the attachment to the π-electron-pair-donating heteroaromatic N of -D;
═X¹, —R¹, —R^{1a}, —R², —R^{2a}, —R³, —R⁵ and n are used as defined in claim 1;
optionally, one or more of the pairs —R¹/—R^{1a}, —R²/—R^{2a}, two adjacent —R² are joined together with the atom to which they are attached to form a C₃₋₁₀ cycloalkyl, 3- to 10-membered heterocyclyl or an 8- to 11-membered heterobicyclyl;
optionally, one or more of the pairs —R¹/—R², —R¹/—R⁵, —R²/—R⁵ and —R⁴/—R⁵ are joined together with the atoms to which they are attached to form a ring -A-;
wherein -A- is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl;
optionally, —R¹ and an adjacent —R² form a carbon-carbon double bond provided that n is selected from the group consisting of 1, 2, 3 and 4;
optionally, two adjacent —R² form a carbon-carbon double bond provided that n is selected from the group consisting of 2, 3 and 4;
and wherein the distance between the nitrogen atom marked with an asterisk and the carbon atom marked with an asterisk in formula (Ix) is 5, 6 or 7 atoms and if present the carbon-carbon double bond formed between —R¹ and —R² or two adjacent —R² is in a cis configuration.

20. The conjugate or pharmaceutically acceptable salt thereof of claim 19, wherein —R⁵ of formula (Ix) is methyl.

21. The conjugate or pharmaceutically acceptable salt thereof of claim 19 or 20, wherein —R¹ and —R^{1a} of formula (Ix) are both —H.

22. A pharmaceutical composition comprising the conjugate or pharmaceutically acceptable salt thereof of any one of claims 1 to 21.

23. The conjugate or pharmaceutically acceptable salt thereof of any one of claims 1 to 21 or the pharmaceutical composition of claim 22 for use as a medicament.

24. The conjugate or pharmaceutically acceptable salt thereof of any one of claims 1 to 21 or the pharmaceutical composition of claim 22 for use in a method of treating a disease that can be treated with D-H.

25. A method of preventing a disease or treating a patient suffering from a disease that can be prevented or treated with D-H, comprising administering an effective amount of the conjugate or the pharmaceutically acceptable salt thereof of any one of claims 1 to 21 or the pharmaceutical composition of claim 22 to the patient.
